SEQUENCE LISTING (43 sequences)

SEQ ID NO: 1 - protein, 221 aa, Artificial Sequence
  Myomaker - Human
MGTLVAKLLLPTLSSLAFLPTVSIAAKRRFHMEAMVYLFTLFFVALHHACNGPGLSVLCFMRHDILEYFSVYGTALSMWVSLMALADFDEPKRSTFVMFGVLTIAVRIYHDRWGYGVYSGPIGTAILIIAAKWLQKMKEKKGLYPDKSVYTQQIGPGLCFGALALMLRFFFEDWDYTYVHSFYHCALAMSFVLLLPKVNKKAGSPGTPAKLDCSTLCCACV

SEQ ID NO: 2 - protein, 221 aa, Artificial Sequence
  Myomaker - Dog
MGTLAAKLLLPTLSSLAFLPTVSIAAKRRFHMEAMVYLFTMFFVALHHACNGPGLSVLCFMRHDVLEYFSVYGTALSMWVSLMALADFDEPKRSTFVMFGVLTIAVRIYHDRWGYGVYSGPIGTAVLIIATKWLQQMKEKKSLYPDKSVYTQQIGPGLCFGALALMLRFFFEDWDYTYVHSFYHCALAMSFVLLLPKVNKKAGSAGPPAKLDCSTLCCACI

SEQ ID NO: 3 - protein, 221 aa, Artificial Sequence
  Myomaker - Pig
MGTVMAKLLLPTLSSLAFLPTVSIAAKRRFHMEAMVYLFTTFFVAFYHACHGPGLAMICFLRLDILEYFSVYGTALSMWVSLMALADFDEPKRSTFVMFGVLTIAVRIYHDRWGYGVYSGPIGTAALIIAAKWLQQMKDQRRLYPDKSVYTQQIGPGLCFGALALMLRFFFEEWDYTYVHSFYHCALAMSFVLLLPKANKKAGSAGPPAKLDCSTLCCACI

SEQ ID NO: 4 - protein, 221 aa, Artificial Sequence
  Myomaker - Mouse
MGTVVAKLLLPTLSSLAFLPTVSIATKRRFYMEAMVYLFTMFFVAFSHACDGPGLSVLCFMRRDILEYFSIYGTALSMWVSLMALADFDEPQRSTFTMLGVLTIAVRTFHDRWGYGVYSGPIGTATLIIAVKWLKKMKEKKGLYPDKSIYTQQIGPGLCFGALALMLRFFFEEWDYTYVHSFYHCALAMSFVLLLPKVNKKAGNAGAPAKLTFSTLCCTCV

SEQ ID NO: 5 - protein, 221 aa, Artificial Sequence
  Myomaker - Opossum
MGTLVTKLLLPTISSLAFLPTISIAAKRRFHMEAMVYLFTMFFIAIYHACDGPGLSVLCFMRYDILEYFSIYGTALSMWVSLMALAEFDEPKRSTFVMFGVLTIAVRIYQDRWGYGVYSGPIGTAVLIIATKWLQKMKEKKGLYPDKSVYTQQIGPGFCFGALALMLRFFFQEWDYTYVHSFYHCSLAMSFVLLLPKVNKKAGNAGTPAKLDCSTLCCACI

SEQ ID NO: 6 - protein, 220 aa, Artificial Sequence
  Myomaker - Zebrafish
MGAFIAKMLLPTISSLVFVPAASVAAKRGFHMEAMVYFFTMFFTAIYHACDGPGLSILCFMKYDILEYFSVYGTAISMWVTLLALGDFDEPKRSSLTMFGVLTAAVRIYQDRLGYGIYSGPIGTAVFMITVKWLQKMKEKKGLYPDKSVYTQQVGPGCCFGALALMLRFYFEEWDYAYVHSFYHVSLAMSFILLLPKKNRYAGTGRNAAKLNCYTLCCCV

SEQ ID NO: 7 - DNA, 666 bp, Artificial Sequence
  Myomaker - Human cDNA
atggggacgctggtggccaagctgctcctgcccaccctcagcagcctggccttcctccccactgtcagcatcgcggccaagaggcggttccacatggaggccatggtctacctcttcaccctgttcttcgtggcgctccaccatgcctgcaatggacccggcttgtctgtgctgtgcttcatgcgtcacgacatcctggagtatttcagtgtctacgggacagccctgagcatgtgggtctcgctgatggcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtggctacagaagatgaaggagaagaagggcctgtacccagacaagagcgtctacacccagcagataggccccggcctctgcttcggggcgctggccctgatgctacgcttcttctttgaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctga

SEQ ID NO: 8 - DNA, 666 bp, Artificial Sequence
  Myomaker - Dog cDNA
atggggacgctcgcggcgaagctgctcctgcccaccctcagcagcctggccttcctccccaccgtcagcatcgccgccaagcggcggttccacatggaggccatggtctacctcttcaccatgttcttcgtggcactccaccacgcgtgcaacgggcccgggctatcggtgctctgcttcatgcgccacgacgtcctggagtacttcagcgtctatgggacggcactgagcatgtgggtctcgctgatggcactggctgacttcgacgaacccaagaggtcgacttttgtgatgtttggcgtcctgaccatcgccgtgcggatctaccatgaccgctggggctacggggtgtactcgggccccattggcacggctgtcctcatcatcgccacaaagtggctgcagcagatgaaggagaagaagagtctgtacccggacaagagtgtctacacccagcagataggccctggcctctgttttggggcactggcccttatgctgcgcttcttttttgaggactgggattacacctatgtccacagcttctaccactgtgccctggccatgtccttcgtcctcctgctccccaaggtcaacaagaaggctggaagcgcggggccccctgccaagctagactgctctaccctttgctgtgcttgcatctga

SEQ ID NO: 9 - DNA, 666 bp, Artificial Sequence
  Myomaker - Pig cDNA
atggggaccgtcatggccaaactgctgctacccacgctgagcagcctggccttcctccccacggtcagcatcgctgccaagcggcggttccacatggaggccatggtctatctcttcaccacgttcttcgtggcgttctaccacgcctgccacgggccgggcctggctatgatctgctttctgcgccttgacatcctggagtatttcagcgtctacggaaccgccctgagcatgtgggtctcgctgatggcgctggctgacttcgacgagcccaagaggtcgactttcgtgatgtttggcgtcctgaccatcgccgtgcggatctaccacgaccgctggggctacggcgtgtactcgggccccatcggcacggccgccctcatcatcgcggccaagtggctgcagcagatgaaggaccaacggcgcctgtatccagacaagagcgtgtacacacagcagataggccccggcctctgcttcggggcgctggccctcatgctgcgctttttcttcgaggagtgggattatacctacgtccacagcttctaccactgcgccctggccatgtccttcgtcctgctgctgcccaaggccaacaagaaggctggaagcgcagggccacccgccaagctggactgctccaccctctgctgtgcttgtatctga

SEQ ID NO: 10 - DNA, 666 bp, Artificial Sequence
  Myomaker - Mouse cDNA
atggggacagttgtagccaaactgctcctgcctaccctcagcagcctggccttcctcccgacagtgagcatcgctaccaagaggcgtttctacatggaggccatggtctacctcttcaccatgttctttgtggcgttctcccatgcctgtgatgggcctggtttgtctgtgctgtgcttcatgcgccgtgacattctggagtacttcagcatctatggaacagccctgagcatgtgggtctccctgatggcactggccgactttgatgaaccccagagatcgaccttcacaatgcttggcgtccttaccatcgctgtgcggacttttcatgaccgctggggttacggggtatactccggtcccataggcacggccaccctcatcattgctgtaaagtggctgaagaagatgaaagagaagaagggcctgtaccccgacaagagcatctacacccagcagataggccccggcctgtgctttggggccctggccctgatgcttcgattcttctttgaggaatgggattacacctacgtccacagcttctaccactgtgccctggccatgtcctttgtcctgctgctgcccaaggtcaacaagaaggctgggaacgcaggggcccccgccaagctgaccttctccaccctctgctgcacttgtgtctga

SEQ ID NO: 11 - DNA, 666 bp, Artificial Sequence
  Myomaker - Opossum cDNA
atggggactcttgttaccaagttgcttcttcccacaatcagcagcctcgcctttctccccaccatcagcatcgctgctaagaggagattccacatggaagccatggtctacctcttcaccatgttcttcatagcaatatatcatgcatgtgacgggccaggcttatcagtgctatgcttcatgcgctatgacatactggagtatttcagcatctatgggacagcactgagcatgtgggtgtcattaatggcactggcagagttcgatgaaccaaaaaggtcaacctttgtaatgtttggcgtgttgactattgccgtgaggatctaccaagaccggtggggatatggggtatactcggggcctattggcacagctgtccttatcattgcaacaaaatggctgcaaaagatgaaagagaagaagggtctgtaccctgacaagagtgtgtacacccaacagataggccctggtttctgttttggagcgttagcactgatgctgcgtttctttttccaggagtgggattacacctatgttcacagcttctaccactgttcactagccatgtcctttgtcttgctgctgcccaaggtaaacaagaaagctgggaatgctgggacacctgccaaattggactgttctacactctgctgtgcttgcatctga

SEQ ID NO: 12 - DNA, 663 bp, Artificial Sequence
  Myomaker - Zebrafish cDNA
atgggagcgtttatcgccaagatgttgctgcccactattagcagtttggtgtttgtgcctgcagccagcgtggctgcaaagaggggcttccacatggaggccatggtctatttcttcacaatgttcttcaccgcgatttaccacgcatgtgacggtccgggcttgtccattctctgtttcatgaagtatgacattctggagtacttcagcgtgtacgggacagccatctccatgtgggtcacgctactggcgcttggggatttcgatgagcccaaacgctcttcgctcaccatgtttggggtgttgaccgcagctgtgaggatttaccaggaccgactgggctacggcatttactccggccccatcgggacagctgtctttatgatcacagtcaaatggttacagaaaatgaaggaaaagaaaggcctttatccagacaaaagtgtttacactcaacaagtgggcccagggtgctgcttcggtgctcttgctttgatgcttcgcttctattttgaggagtgggactacgcttatgttcacagtttctaccacgtgtctctggccatgtcctttattctgctgctgcccaagaagaaccgttatgctggaacgggacgtaacgcagccaaactcaactgctacaccctctgctgctgtgtatga

SEQ ID NO: 13 - DNA, 10361 bp, Artificial Sequence
  Myomaker - Human (+strand)
caagtgtgagctggggagggcaggggctcagagccgggctgggcgcagcatcagacacaagcacagcacagggtggagcagtccagcttggccggggtccccggggatccagccttcttgttgaccttgggcagcagcagaacaaaggacatagccagggcacagtggtagaagctgtggacataagtgtagtcccagtcctgcggggggcaagcggtcagtctggggcctcagccccctccccgaggctcctccctctccaagacccagcagagccccttcaggcccccgcctctgccagggcactgggacacctgcaggaagcctcccccacggtcgcgctcacagtggtttttctctccacctaaacccagagcagtgagggcctgtgccatcctccaggctgcactccttccttcttccccatcccctctctctgctgtccttctcttcctccatccttctctccctcctaccctccctccctccatctccccctcttttctctccttatccctcttcccctgttcctccctccctcctccactttctccctccttccttccctgtctcctcccctccctccctccctcctccaggtgttgggcacctgccccaggcgtctcccaggctgtgctgccgtctgagatgccagctgtctgtaggcagccagctttggtctctgtgacctccaggtccacacaggccatggtgctggtggtgctggggacggcattgcccccgacatagccctgggaggggctagtgagcagggactaataccagactttggcctggggctgtcagagtccccccagcgtgggcacagccctggtatcccagctgagcagagccatgccgagtgggctctggggcacaggacacctccccgctgggcttggtacctcaaagaagaagcgtagcatcagggccagcgccccgaagcagaggccggggcctatctgctgggtgtagacgctcttgtctgggtacaggcccttcttctccttcatcttctgtagctgtgaggacaggaggccacagcaaagcttttaggtcacagcactggggaacgcccctccccaaaccagcccgagagctggccctgcacaggctcaccccagccctctcccggcaggagaggaggctcaggagcctcctgccgcacccagcctcagatggcttctgctggacagggcccttcacggtgcgacccagcagagaccccagcctggatggctgggaaggaagccactgggccatgtgccccacaaagaccccgctgccctcccgcctctttgagatgtaacaacgccaccctcgcatgtctcctcctccctggaggggagctctggggggactagactccatgattgcttaccaaggaaagtactggagtacttgggacctgccagcccagtgtggcccatggggatggcacttgtggtgatccctgagccatggacaagcatcgtttgctttcctagttaaaggacctatctcactcttcattagacaaacttggccagcactgcttctcaggtcccagtgcttaggaaggctcgcgtgggcgtttccacttacagaggggtttgcattccgaggaagatgcgggaagtgtggggccacatccctggagccggccttgtgttttctaggccacttcacatggagtctatttgggattttcaagggcagttgtttcctggaatgagggtggatttttctccctgagcctggtcccctcttgggaggggctggggaacgacagccttgttggggaggaaggagggagggttgggtgatggcggcctcggagtggggccagacccgtgggggtacactcaggaggctatagatttcagtggaatcaactgttagacacacagcgtgtggcacaagcccctgggggtgggggcagcaccccataactgcacccattgctgagtggcctatgcaaagagcacaaagagccttatgctgggtcaggtcaggttttgccacccagtgaattatgaattgatgcccggctttccattttctggaattccattgccaacaaggaattgagcacctgcagtcctgcagtggcctgaagacagctggaccgtgtgaccctgggtgcggtggtcaaggctgccagcccacctctggccagccctgcagtagtaacaccagggagaagagaggtgcctgccccaggtcacacagtgggcctggcactattgaaagggcgccatcacccaaccctccctccttcttcctcccgggctgccattgcccaacccctcccaagaggggacgaggctcagggagaaaaatccaccctcattccaggaaacaattgcccttgaaaatcctaaataaactccatactaaatggtctagaagacaacaatttgagccccagatgcggggaggcgggcagcccatcctcggctcctgtggctggatctgcagcctgagggccttggcagtctcgtggctcttggtgggaaacacagcagtgaattctcttctgggcaattacagttcagcccagttcagacctggccaagaccagcgggaggagcaaccttcaggggcagaaggaggcgagaggcgggtggccaggacccagggccccagcacgctccttcctgccacccaccttggtccagcccacttatgcccagcgctccctctctccccaccaggtgactcccaggggcctcctgggtcagcccaggattagtgctgcttcctcaggttgcagacagaaagcaggtcctctgtctcctgctcaaaaagtcaagtccagccaggcgtggtggctcatgcctgtaattccagcactttgggagactgaggcaggcagattacctgaagtcaggagctcaggaccagccgggccaacgtggtgaaaccccatcgctactaaaaatataaaaattacctgggcgtgatggcatgcgcctataatcccagctactcgggaggctgagacaggagaatcgcttcaacccgggaggcggaggttgcagtgagccaagatggcgccattgcactccagcctgggtgacaagagcaaaactccgtctcaaaaaaaaaaaaaaaaaagtcaggttctggccccgccactgccctgccatgacgtcctgttaagttgctgaggcctccatgctttggttccttcataggccaaatggcaaatcagtcccatgctccttggctgtggggaggattgggacgggctttgcaagctgcccaccagaactcgagcgctctccccacagccgtgggccctcctgcactgagagctgccctctgtcttgctgggtgtcctgcggctctggccggggctggcagtgtggctgggctggaccaggccaggtcctctcttggcacttgaaactgaccctgagacttcaggtccactccaaagaggtgaaatgcagcacagggatgttcaggcggtgcctgggctgctgcaggcctggagagcaggctcaggctgaagcctgctggctccccaggtctgggagacccttgcaagggtgagctccctcctgctctggggtcccaggagatgccccgggtctatttttccctaagatccctctttagcttgggcgagtttgagtggggtttggtccctgagccaggagggtcttggtaggacggagagagcagggagcactgaagaccacgtgagggccttgctgctctgcaaggggctgtctgtgctagaaggtctggcccaggctgcctcactgtcataccacactctccctcctggctagaaccaagctcgaggctcactccctccaggaagtcttcccagattaccccaggccattttccaagttgatgttgcatctctaaagcagctggtagtaagagcggtgatgagagtgataacaaatagctcttatgtgcggagcacattggaagccaggctccatgccaggacttcaggtgcctgatctcagtgagtctttgaaccaccccatgagacaggcagggggctgtaatgacaacacctgctttacaggtacgggcgtggaggtgagacattgggtaacttgggctcagtctggagctggtgagtacagacaagcgtcacacacagtctacacagccggagcacctcatggctattttctacgtggttttgctgaattcctgcatccacccatttgcctatgagggcaggaggtaaatgaagatccgaggcaggaggagtcagacaggggagaggtgacgggcctcctgggtccccgttcatcgaggctcgcgcagtacgcacccactttgccgcgatgatgaggatggctgtgccgatggggcccgagtacaccccgtagccccatcggtcatggtagatccgcacagcaatggtcaggacgccgaacatcacaaatgttgacctcttgggttcgtcgaagtcggccagtgctggaggggccagggagacacagggggaggtgagtggtctctcttgctcctcctggctacccccccaccccccagcccccaggaggcatcctgtagatgccctctctcggtgtcccctcagccagcgagaccctgaggcccagcctggtcatggaggggtctgaattccagccagtttgagaggacaggcagcctgctgcttccccatggacacagcagcttggattgtgctcccagcacctcattttaataaacagaccacagctggttgtggtggctcaggtctgcaatcccagtgctttgggaggcagaggcaggaggatcgtctgagaccaggagttcaagactagcctgggcaacatagcgggacccccatctccacaaaaaattcggtgggtgtcgtggtgcatgcctgtcatcccagctacttgggaggctgaggtgggaggatggcttgaggctgtgagttcgaggctgcagtgagccgtgtttgtgccactgtactctggcctgagtgacagagtgagaccctgtggctaaaaatcaataatcactatgcaaagtgaataggatcgaatctatcccataggatcacaggacaaagacactaagattcaagagaagaaatgaagcccctcacaggcccggttagatggcaaggagcctcaggtcatggggaccttgccacagacaacagttacgtggaaaaaaacatggtgggaaagggggcttatgaacagtcccgtcttccaggctggatatcacccgtgtgtgtggatgtttgtatgacagtctgggaagccaacccccctgagcagtgaacagcggtcctcccagggaaggagtgacgggagggagccctttcactttttcctttgtatgcctctgctgttgaaatgtgtcacaacaagcttttactaaatgagtcattttaaaaggatataaaaaatcggcatcagggcatttaagaggtgcatattctttttcatagattaagcacaaccctgaaacccagacaagggaagacattcctggggctgggagtgagtggggatagagggctgcagcgggactggtttgaggctgggtgtgcggacactggggagccggtccttgtccgcaaggcttgtctgcaggggttgaccactcacccatcagcgagacccacatgctcagggctgtcccgtagacactgaaatactccaggatgtcgtgacgcatgaagcacagcacagacaagccgggtccattgcaggcatggtggagctgccagaaaacccacaggtggtcacagcacaaagaggccagagctggtccccgagccacggcccccagagtgccaggtcacttgctggctgtgagaagtcactttggcgagtcacttaatgactgtgtgcctcagtctccccgtctgaaaaatgggggtactgccgagcactcccgcagagggtcctgtggggattaagtggcacatgccagcgaggtgtttaggggctggggtgtgccaagggttcactcaatgtcacctcagcagaattcgctcatctgcactggcaggactgggcggagactgagtggtcactcaggtgaagcccgcttaggtggggcggtctccgggagggaccctacacggctctccccggaccttcagcatctgtgcttccttgaagcacacagctgcgtgttcactcgccaatctttggatgtgaggtcagagcctctctgggggctcctttgctctttgggggctcctggggccttctcttgcacaaattacccctctgatgactggtctacactgcagcagcgttctcaggcttgagtgggcatcagaacgcctggggccttgtttagacacaggttactgagccctgcctagggttgctgattagggagggctgggttgggtagaaaatgtgcattttgaacacattccctgtggcactgctgaggctggcagggcccacactgagagccgggctgtagctcctggtttctgttgccttaacgtggacgaagatctctgagacccccttgcagaagctgaacacagccccctaggctcatccatctctgccctatactctcgtcgtcgcctccccaacacccactttcatggcaatttttaaggcaaaaggcttatagggagtgttttcaaagcagtcaactacttttctacggaaaacaactctctctccttttgcattcgcatttcatcattttaggtaatatttaattacatgacataattattttgacaggttcaactggcacaaacaagcttgggaaacagcacggtggactcttggtcagcccagctcagcgggaggagcaggcgtgctggaaagcagcccgtgtctggaggcgacagggacagcacagagggagcgggggccctgggtgatctggggggcaggcaattcggggtcaaagtggagtgcttctactgatggcaattgtacacggcctaaagtgacggtgcacctaggaggcattaatagggatccagcatctaaaatgagggaggcggcggtcctgcttctctctgttcttgtcaggctcatccagaagactatgccgagctctgtgtggtgcacctttcttgaagtgagactgggaaagacgcggctagaggagggtgaccagcggtggacatgactgtttaccttggggacagggaagcttcaggaggggcctgatcaaggtgcttacacctctgtgggaaagaggagcgaggaagactccggccctaggctgttctcctgttctcctggcttcttcccatcccccaccccagccccatcacctgctgtctgtgtgcctcaatgtagcacagatggtcatgtgtgattaaggcattcactgtgagattgtgataaggcctgtgcccttgccctgccaggagcaggaatggctctgtctggtcccagttgcatggacggctcccagcatagagtgcttgctgcatgtgttcagggagggggacgccaggctctgagaattctaaaggacagccagctcaccctggggacccagagcctctgccactaggcccttggctcctcccaatggtgggaacttagctccattcgacagatggggaaagtgaacttcagagcagcactgcctgcccaaagaggtgaaacagagcagtgcttggcacctggccacttcctcccatcctgcagtgcagggggcagacctggcccagccggggcactggtggggtgggtgcggctgagggcctggggggtcagagctcaggctcggggagtctgactttgcagatgttcccagtgggggctcaggtgagtggctgtcgggggggggcctcctctgttgtgtggggacaagcacactgtctccgtggggtttgcacccatagcaaggtgtccggcacagagatggagattgtcacgggaggggcctgattggaagggaagggacgccatgcgggtggcagaactttgggagggactgagtgtggctttgagttcagaagacgtttgtcacaagaggcagctgcccctgccactctgggtggggcagggtggggcctctgagaccagtgcagaggcagctgcggggccagcctaggcccaggcagggaggtgtggcctggtgggtgcttgtggtttgctgggctaggtctaacaggagccttgagaacaagacctcagcttttctccctgcgctaaggccatgggacctgcagagaaatcctggctctgctctgggcttcagtctctcatctgcccaagaggcttcctagccctagcccaggctggagtcccagaggagcgaatgcagtggcatttgggtgagtcaggagctctggagagcttgatggtcacagtgacacaagtgactctgtctctctgggatttggtttcttcatctgccaaatgggaatcaagatcctaggcttgtggggaaggtgaaaaggctgaatcagacactgtgcacagagcgcctagccgagtcctctgccctgggtactggcgctcgaggtggactcagaagctccagggcatctggttccacaaaggacccagcctgtcccaggccactgtcacccctgggagtggcacacactggagggaatgcctcgctcccagcccacacgtgcacactcagcttctgccattgcgggcaaaattggacttgaccaattcaggatacaagcataacatgtgaatatatgcttgcaaacacacgtgtgagctcacgggcctcacccgctcaggactccctctgtgcactcacatgcacttggcattcttgcccatagaggccctgctgctggagaaggaggctgtctggggagaggaggtggagttttcacaggttgggcccagcactgccccaagaaggaggctagtgggacgcttgcctccccagagcaggtgtcatgctggggattgggctgtcagtgaaggaggggtgtgatggaaggtgagcaaggaaggcttcgggagagcaagaggtggggcaccacttgtgggagtccaggagtgagggcatgttagtggagaaagtcggaaagacccagaggcaagaaggcagggggtaccgagacatataaatgatggctgaatggcgagatggtaatagacgaatagatcacaggtagatggatgcgtagatagagagatagatggagagagagagagagagagagagagagagagagagagacaagctggagaaggtggatagctaaagccagagagacacatggagagtcaggggactaaaaccagggagggtgggaccaagagctttagagagagtgaattccatggggatcgagttccagaaatcaaaagagaaccagacagagagagaaaggaaaaaaagagaaacagagaaaactagacacagaaaaccaatacgagaaacacagagtgaaagagacccagaaagagagagagagaagacaggggagacaggggtcccagaaacagcgacctcagaaacaaggacagatggggttctgggcgctccactgaaagccggataagatcacccaatgacaggtaccaggaaacagagagcaggagagagccagagagagagcaagcggagacagtcagccagccagacacataaatagaaagagaaagacggacccacagagagagaagtaggccccagaagagggagagaccagcaggccctcctgaaccagagcagctccaggattctggaatcagactcactcacccaggccttcacactccctgaaccctgcagaccccttcccaggcctggcttgccccactcatctctgctccatcgtggcctatgggtagagctcgaagagaggtggggaggggaggtggccccatgggcagccgtgggggctttgattagcagctgagaaaaggggcacgctggaagggtttatcctcaactcaatggccctgcttcaccccaggcttggtctcacacaggcagtgatcccagagcaacttcctggcacagatgggaaaactgaggtccagataggggaagggactcccctagtcctctctcttcagtctccagaccccacctgggcctgctgtttcattttcaaatcacttctgctcatcacccaatacaagaacgctgtggacagagagcctctcctctacctccaggatggggcctgtgtgggacttcctcccagcccccagactcaccgccacgaagaacagggtgaagaggtagaccatggcctccatgtggaaccgcctcttggccgcgatgctgacagtggggaggaaggccaggctgctgagggtgggcaggagcagcttggccaccagcgtccccatgggccaggaggaaagcactggctggggtggggagggtgctggtgtcccaggtccccagcacaggagcacgaagtgggaaggccagctccctttgggcagggc

SEQ ID NO: 14 - DNA, 10361 bp, Artificial Sequence
  Myomaker  - Human (-strand, reverse complement)
gccctgcccaaagggagctggccttcccacttcgtgctcctgtgctggggacctgggacaccagcaccctccccaccccagccagtgctttcctcctggcccatggggacgctggtggccaagctgctcctgcccaccctcagcagcctggccttcctccccactgtcagcatcgcggccaagaggcggttccacatggaggccatggtctacctcttcaccctgttcttcgtggcggtgagtctgggggctgggaggaagtcccacacaggccccatcctggaggtagaggagaggctctctgtccacagcgttcttgtattgggtgatgagcagaagtgatttgaaaatgaaacagcaggcccaggtggggtctggagactgaagagagaggactaggggagtcccttcccctatctggacctcagttttcccatctgtgccaggaagttgctctgggatcactgcctgtgtgagaccaagcctggggtgaagcagggccattgagttgaggataaacccttccagcgtgccccttttctcagctgctaatcaaagcccccacggctgcccatggggccacctcccctccccacctctcttcgagctctacccataggccacgatggagcagagatgagtggggcaagccaggcctgggaaggggtctgcagggttcagggagtgtgaaggcctgggtgagtgagtctgattccagaatcctggagctgctctggttcaggagggcctgctggtctctccctcttctggggcctacttctctctctgtgggtccgtctttctctttctatttatgtgtctggctggctgactgtctccgcttgctctctctctggctctctcctgctctctgtttcctggtacctgtcattgggtgatcttatccggctttcagtggagcgcccagaaccccatctgtccttgtttctgaggtcgctgtttctgggacccctgtctcccctgtcttctctctctctctttctgggtctctttcactctgtgtttctcgtattggttttctgtgtctagttttctctgtttctctttttttcctttctctctctgtctggttctcttttgatttctggaactcgatccccatggaattcactctctctaaagctcttggtcccaccctccctggttttagtcccctgactctccatgtgtctctctggctttagctatccaccttctccagcttgtctctctctctctctctctctctctctctctctctctccatctatctctctatctacgcatccatctacctgtgatctattcgtctattaccatctcgccattcagccatcatttatatgtctcggtaccccctgccttcttgcctctgggtctttccgactttctccactaacatgccctcactcctggactcccacaagtggtgccccacctcttgctctcccgaagccttccttgctcaccttccatcacacccctccttcactgacagcccaatccccagcatgacacctgctctggggaggcaagcgtcccactagcctccttcttggggcagtgctgggcccaacctgtgaaaactccacctcctctccccagacagcctccttctccagcagcagggcctctatgggcaagaatgccaagtgcatgtgagtgcacagagggagtcctgagcgggtgaggcccgtgagctcacacgtgtgtttgcaagcatatattcacatgttatgcttgtatcctgaattggtcaagtccaattttgcccgcaatggcagaagctgagtgtgcacgtgtgggctgggagcgaggcattccctccagtgtgtgccactcccaggggtgacagtggcctgggacaggctgggtcctttgtggaaccagatgccctggagcttctgagtccacctcgagcgccagtacccagggcagaggactcggctaggcgctctgtgcacagtgtctgattcagccttttcaccttccccacaagcctaggatcttgattcccatttggcagatgaagaaaccaaatcccagagagacagagtcacttgtgtcactgtgaccatcaagctctccagagctcctgactcacccaaatgccactgcattcgctcctctgggactccagcctgggctagggctaggaagcctcttgggcagatgagagactgaagcccagagcagagccaggatttctctgcaggtcccatggccttagcgcagggagaaaagctgaggtcttgttctcaaggctcctgttagacctagcccagcaaaccacaagcacccaccaggccacacctccctgcctgggcctaggctggccccgcagctgcctctgcactggtctcagaggccccaccctgccccacccagagtggcaggggcagctgcctcttgtgacaaacgtcttctgaactcaaagccacactcagtccctcccaaagttctgccacccgcatggcgtcccttcccttccaatcaggcccctcccgtgacaatctccatctctgtgccggacaccttgctatgggtgcaaaccccacggagacagtgtgcttgtccccacacaacagaggaggcccccccccgacagccactcacctgagcccccactgggaacatctgcaaagtcagactccccgagcctgagctctgaccccccaggccctcagccgcacccaccccaccagtgccccggctgggccaggtctgccccctgcactgcaggatgggaggaagtggccaggtgccaagcactgctctgtttcacctctttgggcaggcagtgctgctctgaagttcactttccccatctgtcgaatggagctaagttcccaccattgggaggagccaagggcctagtggcagaggctctgggtccccagggtgagctggctgtcctttagaattctcagagcctggcgtccccctccctgaacacatgcagcaagcactctatgctgggagccgtccatgcaactgggaccagacagagccattcctgctcctggcagggcaagggcacaggccttatcacaatctcacagtgaatgccttaatcacacatgaccatctgtgctacattgaggcacacagacagcaggtgatggggctggggtgggggatgggaagaagccaggagaacaggagaacagcctagggccggagtcttcctcgctcctctttcccacagaggtgtaagcaccttgatcaggcccctcctgaagcttccctgtccccaaggtaaacagtcatgtccaccgctggtcaccctcctctagccgcgtctttcccagtctcacttcaagaaaggtgcaccacacagagctcggcatagtcttctggatgagcctgacaagaacagagagaagcaggaccgccgcctccctcattttagatgctggatccctattaatgcctcctaggtgcaccgtcactttaggccgtgtacaattgccatcagtagaagcactccactttgaccccgaattgcctgccccccagatcacccagggcccccgctccctctgtgctgtccctgtcgcctccagacacgggctgctttccagcacgcctgctcctcccgctgagctgggctgaccaagagtccaccgtgctgtttcccaagcttgtttgtgccagttgaacctgtcaaaataattatgtcatgtaattaaatattacctaaaatgatgaaatgcgaatgcaaaaggagagagagttgttttccgtagaaaagtagttgactgctttgaaaacactccctataagccttttgccttaaaaattgccatgaaagtgggtgttggggaggcgacgacgagagtatagggcagagatggatgagcctagggggctgtgttcagcttctgcaagggggtctcagagatcttcgtccacgttaaggcaacagaaaccaggagctacagcccggctctcagtgtgggccctgccagcctcagcagtgccacagggaatgtgttcaaaatgcacattttctacccaacccagccctccctaatcagcaaccctaggcagggctcagtaacctgtgtctaaacaaggccccaggcgttctgatgcccactcaagcctgagaacgctgctgcagtgtagaccagtcatcagaggggtaatttgtgcaagagaaggccccaggagcccccaaagagcaaaggagcccccagagaggctctgacctcacatccaaagattggcgagtgaacacgcagctgtgtgcttcaaggaagcacagatgctgaaggtccggggagagccgtgtagggtccctcccggagaccgccccacctaagcgggcttcacctgagtgaccactcagtctccgcccagtcctgccagtgcagatgagcgaattctgctgaggtgacattgagtgaacccttggcacaccccagcccctaaacacctcgctggcatgtgccacttaatccccacaggaccctctgcgggagtgctcggcagtacccccatttttcagacggggagactgaggcacacagtcattaagtgactcgccaaagtgacttctcacagccagcaagtgacctggcactctgggggccgtggctcggggaccagctctggcctctttgtgctgtgaccacctgtgggttttctggcagctccaccatgcctgcaatggacccggcttgtctgtgctgtgcttcatgcgtcacgacatcctggagtatttcagtgtctacgggacagccctgagcatgtgggtctcgctgatgggtgagtggtcaacccctgcagacaagccttgcggacaaggaccggctccccagtgtccgcacacccagcctcaaaccagtcccgctgcagccctctatccccactcactcccagccccaggaatgtcttcccttgtctgggtttcagggttgtgcttaatctatgaaaaagaatatgcacctcttaaatgccctgatgccgattttttatatccttttaaaatgactcatttagtaaaagcttgttgtgacacatttcaacagcagaggcatacaaaggaaaaagtgaaagggctccctcccgtcactccttccctgggaggaccgctgttcactgctcaggggggttggcttcccagactgtcatacaaacatccacacacacgggtgatatccagcctggaagacgggactgttcataagccccctttcccaccatgtttttttccacgtaactgttgtctgtggcaaggtccccatgacctgaggctccttgccatctaaccgggcctgtgaggggcttcatttcttctcttgaatcttagtgtctttgtcctgtgatcctatgggatagattcgatcctattcactttgcatagtgattattgatttttagccacagggtctcactctgtcactcaggccagagtacagtggcacaaacacggctcactgcagcctcgaactcacagcctcaagccatcctcccacctcagcctcccaagtagctgggatgacaggcatgcaccacgacacccaccgaattttttgtggagatgggggtcccgctatgttgcccaggctagtcttgaactcctggtctcagacgatcctcctgcctctgcctcccaaagcactgggattgcagacctgagccaccacaaccagctgtggtctgtttattaaaatgaggtgctgggagcacaatccaagctgctgtgtccatggggaagcagcaggctgcctgtcctctcaaactggctggaattcagacccctccatgaccaggctgggcctcagggtctcgctggctgaggggacaccgagagagggcatctacaggatgcctcctgggggctggggggtgggggggtagccaggaggagcaagagagaccactcacctccccctgtgtctccctggcccctccagcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtgggtgcgtactgcgcgagcctcgatgaacggggacccaggaggcccgtcacctctcccctgtctgactcctcctgcctcggatcttcatttacctcctgccctcataggcaaatgggtggatgcaggaattcagcaaaaccacgtagaaaatagccatgaggtgctccggctgtgtagactgtgtgtgacgcttgtctgtactcaccagctccagactgagcccaagttacccaatgtctcacctccacgcccgtacctgtaaagcaggtgttgtcattacagccccctgcctgtctcatggggtggttcaaagactcactgagatcaggcacctgaagtcctggcatggagcctggcttccaatgtgctccgcacataagagctatttgttatcactctcatcaccgctcttactaccagctgctttagagatgcaacatcaacttggaaaatggcctggggtaatctgggaagacttcctggagggagtgagcctcgagcttggttctagccaggagggagagtgtggtatgacagtgaggcagcctgggccagaccttctagcacagacagccccttgcagagcagcaaggccctcacgtggtcttcagtgctccctgctctctccgtcctaccaagaccctcctggctcagggaccaaaccccactcaaactcgcccaagctaaagagggatcttagggaaaaatagacccggggcatctcctgggaccccagagcaggagggagctcacccttgcaagggtctcccagacctggggagccagcaggcttcagcctgagcctgctctccaggcctgcagcagcccaggcaccgcctgaacatccctgtgctgcatttcacctctttggagtggacctgaagtctcagggtcagtttcaagtgccaagagaggacctggcctggtccagcccagccacactgccagccccggccagagccgcaggacacccagcaagacagagggcagctctcagtgcaggagggcccacggctgtggggagagcgctcgagttctggtgggcagcttgcaaagcccgtcccaatcctccccacagccaaggagcatgggactgatttgccatttggcctatgaaggaaccaaagcatggaggcctcagcaacttaacaggacgtcatggcagggcagtggcggggccagaacctgacttttttttttttttttttgagacggagttttgctcttgtcacccaggctggagtgcaatggcgccatcttggctcactgcaacctccgcctcccgggttgaagcgattctcctgtctcagcctcccgagtagctgggattataggcgcatgccatcacgcccaggtaatttttatatttttagtagcgatggggtttcaccacgttggcccggctggtcctgagctcctgacttcaggtaatctgcctgcctcagtctcccaaagtgctggaattacaggcatgagccaccacgcctggctggacttgactttttgagcaggagacagaggacctgctttctgtctgcaacctgaggaagcagcactaatcctgggctgacccaggaggcccctgggagtcacctggtggggagagagggagcgctgggcataagtgggctggaccaaggtgggtggcaggaaggagcgtgctggggccctgggtcctggccacccgcctctcgcctccttctgcccctgaaggttgctcctcccgctggtcttggccaggtctgaactgggctgaactgtaattgcccagaagagaattcactgctgtgtttcccaccaagagccacgagactgccaaggccctcaggctgcagatccagccacaggagccgaggatgggctgcccgcctccccgcatctggggctcaaattgttgtcttctagaccatttagtatggagtttatttaggattttcaagggcaattgtttcctggaatgagggtggatttttctccctgagcctcgtcccctcttgggaggggttgggcaatggcagcccgggaggaagaaggagggagggttgggtgatggcgccctttcaatagtgccaggcccactgtgtgacctggggcaggcacctctcttctccctggtgttactactgcagggctggccagaggtgggctggcagccttgaccaccgcacccagggtcacacggtccagctgtcttcaggccactgcaggactgcaggtgctcaattccttgttggcaatggaattccagaaaatggaaagccgggcatcaattcataattcactgggtggcaaaacctgacctgacccagcataaggctctttgtgctctttgcataggccactcagcaatgggtgcagttatggggtgctgcccccacccccaggggcttgtgccacacgctgtgtgtctaacagttgattccactgaaatctatagcctcctgagtgtacccccacgggtctggccccactccgaggccgccatcacccaaccctccctccttcctccccaacaaggctgtcgttccccagcccctcccaagaggggaccaggctcagggagaaaaatccaccctcattccaggaaacaactgcccttgaaaatcccaaatagactccatgtgaagtggcctagaaaacacaaggccggctccagggatgtggccccacacttcccgcatcttcctcggaatgcaaacccctctgtaagtggaaacgcccacgcgagccttcctaagcactgggacctgagaagcagtgctggccaagtttgtctaatgaagagtgagataggtcctttaactaggaaagcaaacgatgcttgtccatggctcagggatcaccacaagtgccatccccatgggccacactgggctggcaggtcccaagtactccagtactttccttggtaagcaatcatggagtctagtccccccagagctcccctccagggaggaggagacatgcgagggtggcgttgttacatctcaaagaggcgggagggcagcggggtctttgtggggcacatggcccagtggcttccttcccagccatccaggctggggtctctgctgggtcgcaccgtgaagggccctgtccagcagaagccatctgaggctgggtgcggcaggaggctcctgagcctcctctcctgccgggagagggctggggtgagcctgtgcagggccagctctcgggctggtttggggaggggcgttccccagtgctgtgacctaaaagctttgctgtggcctcctgtcctcacagctacagaagatgaaggagaagaagggcctgtacccagacaagagcgtctacacccagcagataggccccggcctctgcttcggggcgctggccctgatgctacgcttcttctttgaggtaccaagcccagcggggaggtgtcctgtgccccagagcccactcggcatggctctgctcagctgggataccagggctgtgcccacgctggggggactctgacagccccaggccaaagtctggtattagtccctgctcactagcccctcccagggctatgtcgggggcaatgccgtccccagcaccaccagcaccatggcctgtgtggacctggaggtcacagagaccaaagctggctgcctacagacagctggcatctcagacggcagcacagcctgggagacgcctggggcaggtgcccaacacctggaggagggagggagggaggggaggagacagggaaggaaggagggagaaagtggaggagggagggaggaacaggggaagagggataaggagagaaaagagggggagatggagggagggagggtaggagggagagaaggatggaggaagagaaggacagcagagagaggggatggggaagaaggaaggagtgcagcctggaggatggcacaggccctcactgctctgggtttaggtggagagaaaaaccactgtgagcgcgaccgtgggggaggcttcctgcaggtgtcccagtgccctggcagaggcgggggcctgaaggggctctgctgggtcttggagagggaggagcctcggggagggggctgaggccccagactgaccgcttgccccccgcaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctgatgctgcgcccagcccggctctgagcccctgccctccccagctcacacttg

SEQ ID NO: 15 - DNA, 10526 bp, Artificial Sequence
  Myomaker - Mouse (+strand)
ttccaggaactagaatgtatgttaggcgaagctaatgactagtggctgatcaagagtttactgtgaatggcttgatcgaaaacctgcagaagggatgggactcaggcaggggtatgcaaggttcgctggctccagcttcctaagtggagagctttcagagcctgggcaggggttaaaagggcaatcccagtttcctagggaaagcagacgattctgacaggcaggacctgggaaatagataaccctgcatgctgctgggtatttactggtctagggttctctgccaggcacacctatggttgtgaggccttgggggataaagttcttttttttcctgaacagagtgaagcaactggtgaacacagaaccagtgggtccctaagcagcactcagcagaatgcagcaggcctgctggtctcttggggtgtagagaagaccatttctcatgtacaggccgcataacaaagtataggaagtaccttgggagagacagcaggactgccaggcaggaaggcaggggcctggtgtgtgtgtgtgtgtgggggggtatagtcagacacaagtgcagcagagggtggagaaggtcagcttggcgggggcccctgcgttcccagccttcttgttgaccttgggcagcagcaggacaaaggacatggccagggcacagtggtagaagctgtggacgtaggtgtaatcccattcctgtggaggagaatgagtcagtctgggcctccatcccttccctaaaccaagtcctagccatttggtgcctctgtcagccagcccaccctgagaaggtggcagaaaggcttgctgccttcctctgttccatgcctcctgggtgctgggcaccagctcctggttccttccaggacatgcgtgcatcttgggtgcaggcttcctaaagtcagggcctgacttgtccactcaggcagtgaggctagtacactggggatggtgagtaccatcctcaagaggacagaatttacaacttggagcctccatatgtggctgttagttaactatttccagaggctcttgctccccttccccataggccaggtacctcaaagaagaatcgaagcatcagggccagggccccaaagcacaggccggggcctatctgctgggtgtagatgctcttgtcggggtacaggcccttcttctctttcatcttcttcagctgcaggcacaaggtggggacatcaaagttcttggggtgcagcacaggaagggacccctccatgaactgtagaagagccctacccccattcctctgtatgcctgactgatgggactctctgggccaatttcccctgggtcctctactgcccgcatctggtgggctttggcacttcagtggcagacgtgatcagttttcccagctaaggggttttcctctgttaaccttggtttcataggccctgtgtgttcaagcttggtaagatggagtgttacatggaatagatgggagtcccatggttcctcactggaatgcacatccttggggcccaaaggtattttaggtattcaagattgttcaggtttcagtggggaagatcattataaataccactgtcaggtgtgcacagagggcacaggacagcagccctgactgagtgatgtgcacagtgggcacaggacagcggccctaattgcacacctcactaaatacattatatgtacaaatgctgtcaatggcctcgtgcaaatcagggcaagctttgtcactctgagtgatgatatgttgctgtttccaagtgttctaaaacttgccattagtaacaggagtggaggtcccagtgagcagtgccagtgacatgggcaccgcctattagcctgagtgtaggccgtatgaccatcaatcacacagttctaacactggggccccagagaggagaagaatattgaagatcacccatgggccctgtcttgccccgggaacccctatttcccatttcactcagcttcttctccccaaatgttgtattcatgttcctttcctgaaagggtgagacatgggaaagaattgtactccgttctaagaagtaagtccaaaccacctgcctatctaagatctaggagatggggtctgtgccccaggcatgggtggctgcagcccctcactcccattctcaccagagacctggggaggctggcatttagtggaggggggcactggcacatgtatgctatcctggctaattaaaatcccatcaggatgggtgtgctgggcttggacaccagcattcaagaggcagaggcgggcagatctctatgagtttgaagccatccagagatacaaagtgagagtctatctttaaaaacaaacaaacaaacaaacaaacaaacaaacaatcaagtcagatccagaaccagtgaagagcagcaaggggccatgataggcaagacaaagaggcagttatcagagcaagccttcttgtttatgcattccagcttgttaactagccatgcagaagcccaacacctctgccttgggtcagagagggccagcttcggctcctcaaactggagtgggatggaagcttctcccctcgaaagtcaagcacagctgccattacctactagggctgcaggttaggctgctgagctctgtgcatttcaggttcatccttaacttaaaatcagaataagcccgggttcctcggagcccacaggagtaggatgtggcttggaagcttcctccctgactatacctgtccccactttgctgaagatggatcagagctctcccacccctggccctgccactcccctctgacacagacacagacacagacacagacacagacacagacacagacacagacacagacacagacacagacacagacacagacacagacagacacagacacagacagacacagacacagacacagacacagacacagacacagacacagacacagacacagacacagacacagacacagacacacaggcatagacacagacacagacagacacagacacagacacagacacagacacagacacagacacagacacagacacagacacagacacagacacagacacagacgacacagacacagacagacacagacacagacagacacagacacagacagacacagacacagacacagacacagacgacacagacacagacagacacagacacagacacagacacagacgacacagacacagacagacacagacacagacacagacacagacacagacacagacacagacacagacacagacacagatgacacagacacagacgacacagacacagtcacagacacagacacagacgacacagacacagacagacacagacacagacagacacagacacagacgacacagacacagacacagacacagacgacacagacacagacagacacagacacagacacagacagacacagacacagacagacacagacacagacacagacacagacacagacacagacgacacagacacagacagacacagacacagacacagacacagacatagacacagacacagagacacagacacagacaacacagacacagacacagacacagacacagacacagactcagacacagacacagacacagacacagacacagacacagactcagactcagactcagactcagactcagactcagactcagactcagactcagacacacagtcacacagacacacacagacacacacagacacacacacaaaggcacacacacacacaaaggcacacacacacacacacaccccaccgcctgccccaatctgcactgctgtagctctacttccaggaacctgcaagatcccaaatggtgcttcctgcatgaggtagcagacaggtgagaacttgaagcctgagtgctgtctgcttggtctgaagcctgctggcctggaaggtctgtgttttgggcctaactgctctgggtggagctcagaaaacatccctgggtctttcctgctattgggaaatttgttcacgatggctaacttaggtgggttttaggccatggaggtgagagggccttgagaccatagaggggttaggagcctatacagcagagtagatgccaagcgccaggccctcctctaggcctcccactcaataccctgcttcacccccacctcacaccttccttcctcatgagaaccatttccaaggcttgcttctttcgggaagactatccagattaacctatctgctctccaaactggtattatacctgtaagcagtgttgtctctcagaatgataatgatagtgatcttatgttgatgaaaagactgacagtgacagtcatgatgacaaaaggtctcctcagctctgggtatattaaaaatcacacctgtgcctgtgcctgtgcttagaagcattctttatgggtatttggatgcagagcagaggtcaagagaaaaggagttttggctttatccaggaccaataaaccagcagggcatgggacccgagcatgagccaccatttttaggaattttagggtttttggcccaattcttactaattcacctgcatataaggatatggggtataggaccctacataggagaaaccaagatcagggaagaaatgcaggttccgtggtctccgacagtggagatactggaagtactcacccactttacagcaatgatgagggtggccgtgcctatgggaccggagtataccccgtaaccccagcggtcatgaaaagtccgcacagcgatggtaaggacgccaagcattgtgaaggtcgatctctggggttcatcaaagtcggccagtgctggggagaggcatagctatggtgagcagcgtccctcacatggctgtgcctccatccttgggaacctattggtatgtcctctcaatctgtagggccagcctggtttccataaggtctgaattttggttatttggagggagtgggtgatgctgcttccctggagcagggtggctgaaataaactggtagactgagtgaccagcatttcctaggaatcctgagacaaaagtgttaagactaatgattggtgcgcagagctgagtctcaggagggaccccgggactgcatccctgggagacaagggtgagcttgcttggtttctcccttttctctttccttcccttccctttctttcccctttctttgctctctccctccttctctccctgtctcccttcctccctccctccctgtctcctttcctttcttcttccttcatcctttctgctttctacttttctctatctcttccttctttttctttcaaaattttgcagttgctggtaatggaatgaagggcctattgattaccaggcgagcgatctgccattgagctgtatataccccaggtccaaggtgaggattttgaatggtctgccttcctaatacacagagctgagctgacccatgagggcaaatgctcctctgagcctggaggacaagctgggaggctaggtccaggatgcctttggcctctcctttgtatgcttctgttttttaaatgtcacaagtgctaactactggagtcacttaaggatggtggaaatgagagtgcaggcatcagagaaatgtgcatgtctctttaagcagattaagctctgcaaagcagcaaggagggaggatctcagagaggggctgggtactggctggggttcaggactggctcccacccattggccaagatggccacttacccatcagggagacccacatgctcagggctgttccatagatgctgaagtactccagaatgtcacggcgcatgaagcacagcacagacaaaccaggcccatcacaggcatgggagaactgtagggaaatcacatgaggtcagcaggcagtgggcagcccaggagtgggtgagaactggtcccaaggctcaggttcactagctgtgagcccctaatggttttgtacctcagcctcctccctcacactatcagagcccttgtggagattaaacaggtgagtccatctagcctgggagtgcaaaagtctttgtaaatatccctttcagactcagcactggcccaaggctggtgagaagcatgctcagaagggcatccttaaagaccacttacacctttgcccatgactgactgaaagtgtacacattcctatgccagtctttgcataggagccttttatcctggacccctgtctctccataaaagaggaagcccttagattccccccaagcaagtgctgattctgacacactggtttctttcccccatatgccagcaggtgtgtccctgactcgtagttgaatagatttgcttctaagcaaaaggttctatatgcaggatttccaagcagacaacttatttcttgcagaaaacaacttgctctccctttgcttcacatttcatcattttaagtaatatttaattacatgacataattattttgacaagtgcaactggcacaaacaagcccagcagccagcacaatgagctcttggtaagcccaacttagcaggagggagcaggcaagctggaaaacagccttgtctggaggcagcaggggcaccaccgagggaggcaggcggagagctggggaccctggatgatggatgtatcagtcaagcacatagggcctacttagaagctcagagacctcctgctggtcacagttgcacatggactctgtcaatcaatagagagcatccaggggaagggaggaggtggtccagcctctgtgttgggtcagccccagccttgagctttgggttctgcaccttttaaaagggagattggtgaagaggaggttaaccaactaggtatgagctcaggaaaagacaagctttgggttgggccagaccaaggtacgcaagtggagaaggaaaggaactcagctctgggaggggactctgctcttctggctccttacagaaccacatgaccccaccccaccccacctgagcccatcatctgtagcatcttgcttccttctcttgtataggcccccatgaatgagtaaaacttacttactgtgagatcctgggaaacactcatgccttcccccacgaggagaagagcttccttaggcttgatctcaacatagaatacttggctacatgtgaaggccagaggagcaggctttctaacaagggatctaactgtcctcaggccctgaggattaattttttggggggtgggtgacctgtgtgacagtgaacttccctggggaacctcctgcccaaggaggcaggggcaaggctgtgatgtgtaccctttctccccagaggcagggagatctggtccagctggtgccaggctaggacacagctgggtgtgacaggagccctaaccctgctgtcagctcagagctggcagaggggcccaggttctctcaggtctctcaggcccaccttgtctaatggcatgagaacacctgttctgtggggcttacaaggggaccctaacgataactgcggagcatggcaccccacactgcaaaaatgaaatgctgtttaaagtttgctttcattaatcaaactttcccccaacctgaaaccaagttaatatgtgcgttatgggcatttaaacaatgtgcttgccctgggcagaattagctcacctctgggaaaaacaattcaatcgatcttattatgctttgcatttctggtggaggactctagtgagtctttgtgactctttcatgcccgactcagaacagtatatgtttgtgtgagatgtggtgaccaggtctaagaccacgtgtgttagaaacagcaaggtatggagaccatgttgaaagcaaaatgtgggtgtaggctgataatatctgattgtggatttgtgtgctactgagtcaaagggccagagagacagctgtctgctataaaagcctaagactcagatcccattctttttgtccctgtttgttgtgctgttcagcaagtagaaaggatgatattgtctaagattcttagattagaacctgattttagattagatgactatcaggttagaacaggagagggcagaattctttggaatacatcagatccacccgctgtgtaactgacaccaagagtcattcttctattcagcagcagcataccatacaactggtagttgtcatggagagtcctacagcagccacgtggaaggcagaactctgtgaggaacagattgtggctttgaggccagaggacatttgtcataagagacagctggccctgccactctgggtggggtgtggcagggtgggcctccaaggccagtgcagaggcagctgtaggccaattagacccaggcaggcaggggtgacctgattggggctgtgatttgctggactgtatctaacacaggccttgggaacaagaccctggcttatgtccttgaccgtggggtctcatcttggctctgaccttggccaggtctcaagaggaacaaatgacagtgtgggacaaagtactgtggggcagaccaggatctgagtgttcatggtgacactggtggcccagtttctctgagactcagtttcctcttctatcaaattgaaatcactatgttaggctcgtgggtgataatgagtccaaccccaccatggttgctttcttgtgacttatcattggcctaatgtcctcccctactgaagtgaactcaagagccatagagtttccagttccttgggttacctatgggaccaccacaaccaggaggtagacaggtgccaagccctcccccactgttctcagcccacatgcattgtggcttctcccaccactagaaagtcatgccagctgactcaggatatggaacacgcatgtgagcacagatgtgtgagtttgtgggctcactcattgagagccagctggataccttcacatactctatgcccttgccttactgagacctgctgcaggaagggcaggcctaaggagaggatgctagtctctaaaagtttggctctgctctaaggaggagactagcaggctgcttgccaaccctgagcatgtatcctaccagtgtgtgggcctcacaccagacaaactagtgaggcatagtgtgatgagagagaaacgaaggttacagagtggtaaaagagacagtgtgactcctggttagaggatagctgagagggccatcatgagaggtactcagaaggactaaagggcaaagtgagaggaggcctttaagacagagagtagatgggtagatgaatggacagggagagagatggttggttagcagataatagagaaatgatagacagatagacagacagacagatgatggatagacacatagaacaagacaaatgataaatgaatagatgatagacaaaggagatagagagacagaagcaagttgaatgggcaggaagataaagtcaggaagacacagagctctggtcaagaacccaggggagagcagaccagggagaagaggagagtgaactcctcgggggagtgtaactctagaaatcagaaaaaaacaaaaaaaaaaaaccccaaaaaacaaacaaacaaacaaacaaaaaaattggatacaggcaggaagaaggaagagatagagactgggagaaactagacacagaaccagtcaaagaagcagagggagagagacccatggcgggaataaagagaagcagaaacccagacacaaggcttcagcaaagctgggccagtgccagacatgccccgaacgaacgacagaggagtcacccagtactgttgcctgggaacagagtggagaaggaactaagaggcagccagccagctagacacataacaggaagagaaagaaggactcagggagaggctggctcctctcagtgggggtagttccaaattctggagctgcagtcacccaggccctctacctttcctgaacctagtagatccattcctaggcctgctcactcaccttgttcctcctcagctgagcaactcatggaacaacgttggtagaaaggagagagagtctgaggagcaccaggcttgaccttaactgacaccgggctctcatgggcctggcctcagtctcaggtgtcaatcacccccctcaaatgtctggcgcacatggagaaactgaggtccacagaggaagacagattccaggaaccttctcttcccagtcaccacccccactgctcccccagacccagactctttctcttccaaatcctgtttctgcatcacctggcacaggacaatggtggtaaccctcccgtgaggacttcctcctaatttcctccttccacacttaccgccacaaagaacatggtgaagaggtagaccatggcctccatgtagaaacgcctcttggtagcgatgctcactgtcgggaggaaggccaggctgctgagggtaggcaggagcagtttggctacaactgtccccatggaccaggaggaaggcactgactggggagaaggtggtaaaggcccccctggtctccagggcaggaagaaaaagagcccacttctttgcttctccagcagccctgaccgcagctgtggcagcacccacaaggagggcttaagtgctc

SEQ ID NO: 16 - DNA, 10526 bp, Artificial Sequence
  Myomaker - Mouse (- strand, reverse complement)
gagcacttaagccctccttgtgggtgctgccacagctgcggtcagggctgctggagaagcaaagaagtgggctctttttcttcctgccctggagaccaggggggcctttaccaccttctccccagtcagtgccttcctcctggtccatggggacagttgtagccaaactgctcctgcctaccctcagcagcctggccttcctcccgacagtgagcatcgctaccaagaggcgtttctacatggaggccatggtctacctcttcaccatgttctttgtggcggtaagtgtggaaggaggaaattaggaggaagtcctcacgggagggttaccaccattgtcctgtgccaggtgatgcagaaacaggatttggaagagaaagagtctgggtctgggggagcagtgggggtggtgactgggaagagaaggttcctggaatctgtcttcctctgtggacctcagtttctccatgtgcgccagacatttgaggggggtgattgacacctgagactgaggccaggcccatgagagcccggtgtcagttaaggtcaagcctggtgctcctcagactctctctcctttctaccaacgttgttccatgagttgctcagctgaggaggaacaaggtgagtgagcaggcctaggaatggatctactaggttcaggaaaggtagagggcctgggtgactgcagctccagaatttggaactacccccactgagaggagccagcctctccctgagtccttctttctcttcctgttatgtgtctagctggctggctgcctcttagttccttctccactctgttcccaggcaacagtactgggtgactcctctgtcgttcgttcggggcatgtctggcactggcccagctttgctgaagccttgtgtctgggtttctgcttctctttattcccgccatgggtctctctccctctgcttctttgactggttctgtgtctagtttctcccagtctctatctcttccttcttcctgcctgtatccaatttttttgtttgtttgtttgtttgttttttggggttttttttttttgtttttttctgatttctagagttacactcccccgaggagttcactctcctcttctccctggtctgctctcccctgggttcttgaccagagctctgtgtcttcctgactttatcttcctgcccattcaacttgcttctgtctctctatctcctttgtctatcatctattcatttatcatttgtcttgttctatgtgtctatccatcatctgtctgtctgtctatctgtctatcatttctctattatctgctaaccaaccatctctctccctgtccattcatctacccatctactctctgtcttaaaggcctcctctcactttgccctttagtccttctgagtacctctcatgatggccctctcagctatcctctaaccaggagtcacactgtctcttttaccactctgtaaccttcgtttctctctcatcacactatgcctcactagtttgtctggtgtgaggcccacacactggtaggatacatgctcagggttggcaagcagcctgctagtctcctccttagagcagagccaaacttttagagactagcatcctctccttaggcctgcccttcctgcagcaggtctcagtaaggcaagggcatagagtatgtgaaggtatccagctggctctcaatgagtgagcccacaaactcacacatctgtgctcacatgcgtgttccatatcctgagtcagctggcatgactttctagtggtgggagaagccacaatgcatgtgggctgagaacagtgggggagggcttggcacctgtctacctcctggttgtggtggtcccataggtaacccaaggaactggaaactctatggctcttgagttcacttcagtaggggaggacattaggccaatgataagtcacaagaaagcaaccatggtggggttggactcattatcacccacgagcctaacatagtgatttcaatttgatagaagaggaaactgagtctcagagaaactgggccaccagtgtcaccatgaacactcagatcctggtctgccccacagtactttgtcccacactgtcatttgttcctcttgagacctggccaaggtcagagccaagatgagaccccacggtcaaggacataagccagggtcttgttcccaaggcctgtgttagatacagtccagcaaatcacagccccaatcaggtcacccctgcctgcctgggtctaattggcctacagctgcctctgcactggccttggaggcccaccctgccacaccccacccagagtggcagggccagctgtctcttatgacaaatgtcctctggcctcaaagccacaatctgttcctcacagagttctgccttccacgtggctgctgtaggactctccatgacaactaccagttgtatggtatgctgctgctgaatagaagaatgactcttggtgtcagttacacagcgggtggatctgatgtattccaaagaattctgccctctcctgttctaacctgatagtcatctaatctaaaatcaggttctaatctaagaatcttagacaatatcatcctttctacttgctgaacagcacaacaaacagggacaaaaagaatgggatctgagtcttaggcttttatagcagacagctgtctctctggccctttgactcagtagcacacaaatccacaatcagatattatcagcctacacccacattttgctttcaacatggtctccataccttgctgtttctaacacacgtggtcttagacctggtcaccacatctcacacaaacatatactgttctgagtcgggcatgaaagagtcacaaagactcactagagtcctccaccagaaatgcaaagcataataagatcgattgaattgtttttcccagaggtgagctaattctgcccagggcaagcacattgtttaaatgcccataacgcacatattaacttggtttcaggttgggggaaagtttgattaatgaaagcaaactttaaacagcatttcatttttgcagtgtggggtgccatgctccgcagttatcgttagggtccccttgtaagccccacagaacaggtgttctcatgccattagacaaggtgggcctgagagacctgagagaacctgggcccctctgccagctctgagctgacagcagggttagggctcctgtcacacccagctgtgtcctagcctggcaccagctggaccagatctccctgcctctggggagaaagggtacacatcacagccttgcccctgcctccttgggcaggaggttccccagggaagttcactgtcacacaggtcacccaccccccaaaaaattaatcctcagggcctgaggacagttagatcccttgttagaaagcctgctcctctggccttcacatgtagccaagtattctatgttgagatcaagcctaaggaagctcttctcctcgtgggggaaggcatgagtgtttcccaggatctcacagtaagtaagttttactcattcatgggggcctatacaagagaaggaagcaagatgctacagatgatgggctcaggtggggtggggtggggtcatgtggttctgtaaggagccagaagagcagagtcccctcccagagctgagttcctttccttctccacttgcgtaccttggtctggcccaacccaaagcttgtcttttcctgagctcatacctagttggttaacctcctcttcaccaatctcccttttaaaaggtgcagaacccaaagctcaaggctggggctgacccaacacagaggctggaccacctcctcccttcccctggatgctctctattgattgacagagtccatgtgcaactgtgaccagcaggaggtctctgagcttctaagtaggccctatgtgcttgactgatacatccatcatccagggtccccagctctccgcctgcctccctcggtggtgcccctgctgcctccagacaaggctgttttccagcttgcctgctccctcctgctaagttgggcttaccaagagctcattgtgctggctgctgggcttgtttgtgccagttgcacttgtcaaaataattatgtcatgtaattaaatattacttaaaatgatgaaatgtgaagcaaagggagagcaagttgttttctgcaagaaataagttgtctgcttggaaatcctgcatatagaaccttttgcttagaagcaaatctattcaactacgagtcagggacacacctgctggcatatgggggaaagaaaccagtgtgtcagaatcagcacttgcttggggggaatctaagggcttcctcttttatggagagacaggggtccaggataaaaggctcctatgcaaagactggcataggaatgtgtacactttcagtcagtcatgggcaaaggtgtaagtggtctttaaggatgcccttctgagcatgcttctcaccagccttgggccagtgctgagtctgaaagggatatttacaaagacttttgcactcccaggctagatggactcacctgtttaatctccacaagggctctgatagtgtgagggaggaggctgaggtacaaaaccattaggggctcacagctagtgaacctgagccttgggaccagttctcacccactcctgggctgcccactgcctgctgacctcatgtgatttccctacagttctcccatgcctgtgatgggcctggtttgtctgtgctgtgcttcatgcgccgtgacattctggagtacttcagcatctatggaacagccctgagcatgtgggtctccctgatgggtaagtggccatcttggccaatgggtgggagccagtcctgaaccccagccagtacccagcccctctctgagatcctccctccttgctgctttgcagagcttaatctgcttaaagagacatgcacatttctctgatgcctgcactctcatttccaccatccttaagtgactccagtagttagcacttgtgacatttaaaaaacagaagcatacaaaggagaggccaaaggcatcctggacctagcctcccagcttgtcctccaggctcagaggagcatttgccctcatgggtcagctcagctctgtgtattaggaaggcagaccattcaaaatcctcaccttggacctggggtatatacagctcaatggcagatcgctcgcctggtaatcaataggcccttcattccattaccagcaactgcaaaattttgaaagaaaaagaaggaagagatagagaaaagtagaaagcagaaaggatgaaggaagaagaaaggaaaggagacagggagggagggaggaagggagacagggagagaaggagggagagagcaaagaaaggggaaagaaagggaagggaaggaaagagaaaagggagaaaccaagcaagctcacccttgtctcccagggatgcagtcccggggtccctcctgagactcagctctgcgcaccaatcattagtcttaacacttttgtctcaggattcctaggaaatgctggtcactcagtctaccagtttatttcagccaccctgctccagggaagcagcatcacccactccctccaaataaccaaaattcagaccttatggaaaccaggctggccctacagattgagaggacataccaataggttcccaaggatggaggcacagccatgtgagggacgctgctcaccatagctatgcctctccccagcactggccgactttgatgaaccccagagatcgaccttcacaatgcttggcgtccttaccatcgctgtgcggacttttcatgaccgctggggttacggggtatactccggtcccataggcacggccaccctcatcattgctgtaaagtgggtgagtacttccagtatctccactgtcggagaccacggaacctgcatttcttccctgatcttggtttctcctatgtagggtcctataccccatatccttatatgcaggtgaattagtaagaattgggccaaaaaccctaaaattcctaaaaatggtggctcatgctcgggtcccatgccctgctggtttattggtcctggataaagccaaaactccttttctcttgacctctgctctgcatccaaatacccataaagaatgcttctaagcacaggcacaggcacaggtgtgatttttaatatacccagagctgaggagaccttttgtcatcatgactgtcactgtcagtcttttcatcaacataagatcactatcattatcattctgagagacaacactgcttacaggtataataccagtttggagagcagataggttaatctggatagtcttcccgaaagaagcaagccttggaaatggttctcatgaggaaggaaggtgtgaggtgggggtgaagcagggtattgagtgggaggcctagaggagggcctggcgcttggcatctactctgctgtataggctcctaacccctctatggtctcaaggccctctcacctccatggcctaaaacccacctaagttagccatcgtgaacaaatttcccaatagcaggaaagacccagggatgttttctgagctccacccagagcagttaggcccaaaacacagaccttccaggccagcaggcttcagaccaagcagacagcactcaggcttcaagttctcacctgtctgctacctcatgcaggaagcaccatttgggatcttgcaggttcctggaagtagagctacagcagtgcagattggggcaggcggtggggtgtgtgtgtgtgtgtgtgcctttgtgtgtgtgtgtgcctttgtgtgtgtgtctgtgtgtgtctgtgtgtgtctgtgtgactgtgtgtctgagtctgagtctgagtctgagtctgagtctgagtctgagtctgagtctgagtctgtgtctgtgtctgtgtctgtgtctgtgtctgtgtctgagtctgtgtctgtgtctgtgtctgtgtctgtgtctgtgttgtctgtgtctgtgtctctgtgtctgtgtctatgtctgtgtctgtgtctgtgtctgtgtctgtctgtgtctgtgtcgtctgtgtctgtgtctgtgtctgtgtctgtgtctgtgtctgtctgtgtctgtgtctgtctgtgtctgtgtctgtgtctgtctgtgtctgtgtcgtctgtgtctgtgtctgtgtctgtgtcgtctgtgtctgtgtctgtctgtgtctgtgtctgtctgtgtctgtgtcgtctgtgtctgtgtctgtgactgtgtctgtgtcgtctgtgtctgtgtcatctgtgtctgtgtctgtgtctgtgtctgtgtctgtgtctgtgtctgtgtctgtgtctgtgtctgtctgtgtctgtgtcgtctgtgtctgtgtctgtgtctgtgtctgtctgtgtctgtgtcgtctgtgtctgtgtctgtgtctgtgtctgtctgtgtctgtgtctgtctgtgtctgtgtctgtctgtgtctgtgtcgtctgtgtctgtgtctgtgtctgtgtctgtgtctgtgtctgtgtctgtgtctgtgtctgtgtctgtgtctgtgtctgtgtctgtctgtgtctgtgtctatgcctgtgtgtctgtgtctgtgtctgtgtctgtgtctgtgtctgtgtctgtgtctgtgtctgtgtctgtgtctgtgtctgtgtctgtctgtgtctgtgtctgtctgtgtctgtgtctgtgtctgtgtctgtgtctgtgtctgtgtctgtgtctgtgtctgtgtctgtgtctgtgtctgtgtctgtgtcagaggggagtggcagggccaggggtgggagagctctgatccatcttcagcaaagtggggacaggtatagtcagggaggaagcttccaagccacatcctactcctgtgggctccgaggaacccgggcttattctgattttaagttaaggatgaacctgaaatgcacagagctcagcagcctaacctgcagccctagtaggtaatggcagctgtgcttgactttcgaggggagaagcttccatcccactccagtttgaggagccgaagctggccctctctgacccaaggcagaggtgttgggcttctgcatggctagttaacaagctggaatgcataaacaagaaggcttgctctgataactgcctctttgtcttgcctatcatggccccttgctgctcttcactggttctggatctgacttgattgtttgtttgtttgtttgtttgtttgtttgtttttaaagatagactctcactttgtatctctggatggcttcaaactcatagagatctgcccgcctctgcctcttgaatgctggtgtccaagcccagcacacccatcctgatgggattttaattagccaggatagcatacatgtgccagtgcccccctccactaaatgccagcctccccaggtctctggtgagaatgggagtgaggggctgcagccacccatgcctggggcacagaccccatctcctagatcttagataggcaggtggtttggacttacttcttagaacggagtacaattctttcccatgtctcaccctttcaggaaaggaacatgaatacaacatttggggagaagaagctgagtgaaatgggaaataggggttcccggggcaagacagggcccatgggtgatcttcaatattcttctcctctctggggccccagtgttagaactgtgtgattgatggtcatacggcctacactcaggctaataggcggtgcccatgtcactggcactgctcactgggacctccactcctgttactaatggcaagttttagaacacttggaaacagcaacatatcatcactcagagtgacaaagcttgccctgatttgcacgaggccattgacagcatttgtacatataatgtatttagtgaggtgtgcaattagggccgctgtcctgtgcccactgtgcacatcactcagtcagggctgctgtcctgtgccctctgtgcacacctgacagtggtatttataatgatcttccccactgaaacctgaacaatcttgaatacctaaaatacctttgggccccaaggatgtgcattccagtgaggaaccatgggactcccatctattccatgtaacactccatcttaccaagcttgaacacacagggcctatgaaaccaaggttaacagaggaaaaccccttagctgggaaaactgatcacgtctgccactgaagtgccaaagcccaccagatgcgggcagtagaggacccaggggaaattggcccagagagtcccatcagtcaggcatacagaggaatgggggtagggctcttctacagttcatggaggggtcccttcctgtgctgcaccccaagaactttgatgtccccaccttgtgcctgcagctgaagaagatgaaagagaagaagggcctgtaccccgacaagagcatctacacccagcagataggccccggcctgtgctttggggccctggccctgatgcttcgattcttctttgaggtacctggcctatggggaaggggagcaagagcctctggaaatagttaactaacagccacatatggaggctccaagttgtaaattctgtcctcttgaggatggtactcaccatccccagtgtactagcctcactgcctgagtggacaagtcaggccctgactttaggaagcctgcacccaagatgcacgcatgtcctggaaggaaccaggagctggtgcccagcacccaggaggcatggaacagaggaaggcagcaagcctttctgccaccttctcagggtgggctggctgacagaggcaccaaatggctaggacttggtttagggaagggatggaggcccagactgactcattctcctccacaggaatgggattacacctacgtccacagcttctaccactgtgccctggccatgtcctttgtcctgctgctgcccaaggtcaacaagaaggctgggaacgcaggggcccccgccaagctgaccttctccaccctctgctgcacttgtgtctgactatacccccccacacacacacacacaccaggcccctgccttcctgcctggcagtcctgctgtctctcccaaggtacttcctatactttgttatgcggcctgtacatgagaaatggtcttctctacaccccaagagaccagcaggcctgctgcattctgctgagtgctgcttagggacccactggttctgtgttcaccagttgcttcactctgttcaggaaaaaaaagaactttatcccccaaggcctcacaaccataggtgtgcctggcagagaaccctagaccagtaaatacccagcagcatgcagggttatctatttcccaggtcctgcctgtcagaatcgtctgctttccctaggaaactgggattgcccttttaacccctgcccaggctctgaaagctctccacttaggaagctggagccagcgaaccttgcatacccctgcctgagtcccatcccttctgcaggttttcgatcaagccattcacagtaaactcttgatcagccactagtcattagcttcgcctaacatacattctagttcctggaa

SEQ ID NO: 17 - protein, 108 aa, Artificial Sequence
  Myomerger mouse-long
MPEESCTVKLIQLKTGEYRGAGPAMPVPLLPMVLRSLLSRLLLPVARLARQHLLPLLRRLARRLSSQDMREALLSCLLFVLSQQQPPDSGEASRVDHSQRKERLGPQK

SEQ ID NO: 18 - protein, 84 aa, Artificial Sequence
  Myomerger - Mouse (short)
MPVPLLPMVLRSLLSRLLLPVARLARQHLLPLLRRLARRLSSQDMREALLSCLLFVLSQQQPPDSGEASRVDHSQRKERLGPQK

SEQ ID NO: 19 - protein, 84 aa, Artificial Sequence
  Myomerger - Human
MPTPLLPLLLRLLLSCLLLPAARLARQYLLPLLRRLARRLGSQDMREALLGCLLFILSQRHSPDAGEASRVDRLERRERLGPQK

SEQ ID NO: 20 - protein, 84 aa, Artificial Sequence
  Myomerger - Cat
MPAPLLPLLLRTLMSRLLLPATRLARRHLLPLLRRLARRLGSQDVREALLGCLLFILSQSRPPDAEEVSRVAGQERRERLAPPK

SEQ ID NO: 21 - protein, 85 aa, Artificial Sequence
  Myomerger - Rabbit
MPAPLLPLLLRTLLSRLLLPAARLARRHLLPLLRRLAQRLGSQGTREALLGCLLFVLSQRQPPDASGEASRVDPPERKERLGRQK

SEQ ID NO: 22 - protein, 84 aa, Artificial Sequence
  Myomerger - Dog
MPAPLLPLLLRTLVSRLLLPAARLARRHLLPLLRGLARRLGSQEVREALLGCLLFILSQRHPPDAEEASRVAGQERKERLAPPK

SEQ ID NO: 23 - protein, 84 aa, Artificial Sequence
  Myomerger - Elephant
MPVPLLSLLLRALLSRLLLPAARLARQHLLPLLRRLARRLGSQDMRQALLGCLLFVLSQQHPPDAGEASREALSERRGRLAPQK

SEQ ID NO: 24 - DNA, 327 bp, Artificial Sequence
  Myomerger - Mouse (long) cDNA
atgccagaagaaagctgcactgtaaaactaatccagttgaaaactggggagtacagaggtgcaggtcctgccatgcccgttccattgctcccgatggtgcttcgatcgctgctgtcccgcctgctgctgcctgttgcccgcctggcccggcagcacctcctgcccttgctgcgccggctggcccgccgactgagctcccaagacatgagagaggctctgctgagctgtctgctctttgtcctcagccagcaacagccaccggattctggagaggcctccagagtggaccactcccagaggaaggagagattgggcccccagaagtga

SEQ ID NO: 25 - DNA, 255 bp, Artificial Sequence
  Myomerger - Mouse (short)  cDNA
atgcccgttccattgctcccgatggtgcttcgatcgctgctgtcccgcctgctgctgcctgttgcccgcctggcccggcagcacctcctgcccttgctgcgccggctggcccgccgactgagctcccaagacatgagagaggctctgctgagctgtctgctctttgtcctcagccagcaacagccaccggattctggagaggcctccagagtggaccactcccagaggaaggagagattgggcccccagaagtga

SEQ ID NO: 26 - DNA, 255 bp, Artificial Sequence
  Myomerger - Human cDNA
atgcccacgccactgctcccgctgctgcttcgattgctgctgtcctgcctgctgctgcctgctgcccgcctggcccgccaatacctcctgcccctgctgcgccgattggcccgccgcctgggctcccaggacatgcgagaggctttgctgggctgtctgctgttcattctcagccagcgacactcgccagacgctggggaggcctcaagagtggaccgcctggagaggagggagaggttaggcccccaaaagtga

SEQ ID NO: 27 - DNA, 255 bp, Artificial Sequence
  Myomerger - Cat  cDNA
atgcccgctccactgctcccactgctgcttcgaaccctgatgtcccgcttgctgctgcctgccacccgcctggcccgccggcacctcctgcccctcctgcgccgactggcccgccgcctgggctcgcaggatgttcgagaagctttgctgggctgtctgttgttcatcctcagccagagccgcccgcccgacgctgaggaggtctccagagtggctggccaggagaggagggagaggctagctcccccaaaatga

SEQ ID NO: 28 - DNA, 258 bp, Artificial Sequence
  Myomerger - Rabbit  cDNA
atgcctgcccccctgctgccgctgctgctgcgaacgctgctgtcccgtctgctgctgcccgctgcccgcctggcccgccggcacctcctgcccctgctgcgccgactggctcaacgcctgggctcccagggcacgcgcgaggctttgctgggctgtttgctgtttgtcctcagccagagacagccgccagatgcctctggggaggcctccagagtggacccaccggagaggaaggagaggttaggccgccaaaagtga

SEQ ID NO: 29 - DNA, 255 bp, Artificial Sequence
  Myomerger - Dog cDNA
atgcctgctccactgctcccactgctgctgcgaacgctggtgtctcgcctgctgctgcctgctgcccgcctggcccggcggcacctcctgcccctgctgcgtggactggcccgccgcctaggctcgcaggaggttcgagaggctttgctgggctgtctgttgttcatcctcagccagagacatccgccggacgccgaggaggcctccagagtggctggccaggagaggaaggagaggctagctccccccaaatga

SEQ ID NO: 30 - DNA, 255 bp, Artificial Sequence
  Myomerger - Elephant cDNA
atgcccgtcccgctgctctcgctgctgctgcgcgcgctgctgtcccgcctgctgctgcctgctgcccgcctggcccgccagcacctcctgcccctcctgcgccgacttgctcgccgcctgggctcccaggacatgcgacaggctctcttgggatgtctgctctttgtcctcagccagcaacacccgccggacgctggtgaggcctccagagaggccctctcagagaggagagggaggctagccccccaaaagtga

SEQ ID NO: 31 - DNA, 1275 bp, Artificial Sequence
  Myomerger - Human (+strand)
ctgcccggtgagagctgccgtggattggtgggggtaggggactgagaggtcagggagtgtcaggtcagggtggatcaggagccccaaaagaaaaattgagaattgcctggagaagaactcctgctagactgagggagaagggttagggaactccaggggcattgaggctgtgcaagaggagggggtgactagaggaagggaggggccagggagcagtaggaatgcctggagctgggaacggcaagctgtaggtcttggtttactcttgccttggttcagtctccccatctgtgctatggtgagaaccttcctgcctcagctgccttgccaagagaaagggcttcatgaaagcaaaaatgacctacaaattgaggtcaggagcaggaaggtgtaaactgaagggagggggaactcctgcccaccccatgtccttgccaggtgaggcagaaccaggacatgcaagcctaaagtctgtgttgtcttcccaggcactgactcactggccctgccatgcccacgccactgctcccgctgctgcttcgattgctgctgtcctgcctgctgctgcctgctgcccgcctggcccgccaatacctcctgcccctgctgcgccgattggcccgccgcctgggctcccaggacatgcgagaggctttgctgggctgtctgctgttcattctcagccagcgacactcgccagacgctggggaggcctcaagagtggaccgcctggagaggagggagaggttaggcccccaaaagtgaggccacaagtcctggcagcagctgtatccacaaaatgctttcttttggagtaggataatcctggcaccagcactgaccgaagcctgcccagtggacagaagatatagtgagggttgtgcatgagagggatctgccacagacatgcctctccactcccaacagaaatgtctttctggaagaatgccttgcatctagcacaaaactgattattgcccctctgtcctccagcagttcctcccaaagaccactcctaatcacctctggcctcaggcgggaggggaactaacacccacccacccctgccctccctgcaaatgggaacatcaaggttcccagtgcttaactgagggacaagtgacaatttagcagagaggcaagatttgaatccagactgtcttccagactcaggacctaccttaaaataatatctgagttgcttatggaggcagacctgcctgcaaagcccagcactcagcaagtgctcaataaatatttgatttgaattctttc

SEQ ID NO: 32 - DNA, 1275 bp, Artificial Sequence
  Myomerger - Human (- strand, reverse complement)
gaaagaattcaaatcaaatatttattgagcacttgctgagtgctgggctttgcaggcaggtctgcctccataagcaactcagatattattttaaggtaggtcctgagtctggaagacagtctggattcaaatcttgcctctctgctaaattgtcacttgtccctcagttaagcactgggaaccttgatgttcccatttgcagggagggcaggggtgggtgggtgttagttcccctcccgcctgaggccagaggtgattaggagtggtctttgggaggaactgctggaggacagaggggcaataatcagttttgtgctagatgcaaggcattcttccagaaagacatttctgttgggagtggagaggcatgtctgtggcagatccctctcatgcacaaccctcactatatcttctgtccactgggcaggcttcggtcagtgctggtgccaggattatcctactccaaaagaaagcattttgtggatacagctgctgccaggacttgtggcctcacttttgggggcctaacctctccctcctctccaggcggtccactcttgaggcctccccagcgtctggcgagtgtcgctggctgagaatgaacagcagacagcccagcaaagcctctcgcatgtcctgggagcccaggcggcgggccaatcggcgcagcaggggcaggaggtattggcgggccaggcgggcagcaggcagcagcaggcaggacagcagcaatcgaagcagcagcgggagcagtggcgtgggcatggcagggccagtgagtcagtgcctgggaagacaacacagactttaggcttgcatgtcctggttctgcctcacctggcaaggacatggggtgggcaggagttccccctcccttcagtttacaccttcctgctcctgacctcaatttgtaggtcatttttgctttcatgaagccctttctcttggcaaggcagctgaggcaggaaggttctcaccatagcacagatggggagactgaaccaaggcaagagtaaaccaagacctacagcttgccgttcccagctccaggcattcctactgctccctggcccctcccttcctctagtcaccccctcctcttgcacagcctcaatgcccctggagttccctaacccttctccctcagtctagcaggagttcttctccaggcaattctcaatttttcttttggggctcctgatccaccctgacctgacactccctgacctctcagtcccctacccccaccaatccacggcagctctcaccgggcag

SEQ ID NO: 33 - DNA, 1136 bp, Artificial Sequence
  Myomerger - Mouse (+strand)
ccaataacaacacactgtcctcgtttattgactacctgctgcgtaccaagctttgaaagtactcattctttaacgggaagcaagggcttataattttaaggtagacgggacagtttggatttaaataccacctcttagctaaattgtcttgagtctaagtgaaacatcatctcttaactgaccttgatacccgcatttgcaggtccaccctggaggccagagataaggcagagggagctgcagagaggaagggtcaatcaacacaatctgtagcctgctaggagctaggggagtgggaactgttcaggtcagagccctcttgcactcagcccggactgtcttcgcccactgggcagtctgccgtccatgcccgtgcgtgcggaccgacgcctggactaaccggctccaaaagtactttgatgggcgttgctgtttccaggacccgtggcctcacttctgggggcccaatctctccttcctctgggagtggtccactctggaggcctctccagaatccggtggctgttgctggctgaggacaaagagcagacagctcagcagagcctctctcatgtcttgggagctcagtcggcgggccagccggcgcagcaagggcaggaggtgctgccgggccaggcgggcaacaggcagcagcaggcgggacagcagcgatcgaagcaccatcgggagcaatggaacgggcatggcaggacctgcacctgcaaagggaacccgggttttagactgtacctcaggcacgcacctcacctggcaaagcagggtgcgggggtgtggagtcctcccttcagcttatacctctgtactccccagttttcaactggattagttttacagtgcagctttcttctggcatgaaagctggttaaggagttcactcactgttatcacagatgggaagggagcccagggctggaaggtggtggggactgaggctagggccttttccagaacccacttcctttaatccctccctccctttgcatactctgacctgaagcctgaacttcttgccctcctgctcaccagttctaaccggccagtggcagctctcaccagtcagaactgctcagaatcaatttcaggatgcttttgcctgcggtggattcagcatcact

SEQ ID NO: 34 - DNA, 1136 bp, Artificial Sequence
  Myomerger - Mouse (- strand, reverse complement)
agtgatgctgaatccaccgcaggcaaaagcatcctgaaattgattctgagcagttctgactggtgagagctgccactggccggttagaactggtgagcaggagggcaagaagttcaggcttcaggtcagagtatgcaaagggagggagggattaaaggaagtgggttctggaaaaggccctagcctcagtccccaccaccttccagccctgggctcccttcccatctgtgataacagtgagtgaactccttaaccagctttcatgccagaagaaagctgcactgtaaaactaatccagttgaaaactggggagtacagaggtataagctgaagggaggactccacacccccgcaccctgctttgccaggtgaggtgcgtgcctgaggtacagtctaaaacccgggttccctttgcaggtgcaggtcctgccatgcccgttccattgctcccgatggtgcttcgatcgctgctgtcccgcctgctgctgcctgttgcccgcctggcccggcagcacctcctgcccttgctgcgccggctggcccgccgactgagctcccaagacatgagagaggctctgctgagctgtctgctctttgtcctcagccagcaacagccaccggattctggagaggcctccagagtggaccactcccagaggaaggagagattgggcccccagaagtgaggccacgggtcctggaaacagcaacgcccatcaaagtacttttggagccggttagtccaggcgtcggtccgcacgcacgggcatggacggcagactgcccagtgggcgaagacagtccgggctgagtgcaagagggctctgacctgaacagttcccactcccctagctcctagcaggctacagattgtgttgattgacccttcctctctgcagctccctctgccttatctctggcctccagggtggacctgcaaatgcgggtatcaaggtcagttaagagatgatgtttcacttagactcaagacaatttagctaagaggtggtatttaaatccaaactgtcccgtctaccttaaaattataagcccttgcttcccgttaaagaatgagtactttcaaagcttggtacgcagcaggtagtcaataaacgaggacagtgtgttgttattgg

SEQ ID NO: 35 - protein, 59 aa, Artificial Sequence
  Extracellular Myomerger - Mouse
RQHLLPLLRRLARRLSSQDMREALLSCLLFVLSQQQPPDSGEASRVDHSQRKERLGPQK

SEQ ID NO: 36 - protein, 59 aa, Artificial Sequence
  Extracellular Myomerger - Human
RQYLLPLLRRLARRLGSQDMREALLGCLLFILSQRHSPDAGEASRVDRLERRERLGPQK

SEQ ID NO: 37 - protein, 59 aa, Artificial Sequence
  Extracellular Myomerger - Cat
RRHLLPLLRRLARRLGSQDVREALLGCLLFILSQSRPPDAEEVSRVAGQERRERLAPPK

SEQ ID NO: 38 - protein, 60 aa, Artificial Sequence
  Extracellular Myomerger - Rabbit
RRHLLPLLRRLAQRLGSQGTREALLGCLLFVLSQRQPPDASGEASRVDPPERKERLGRQK

SEQ ID NO: 39 - protein, 59 aa, Artificial Sequence
  Extracellular Myomerger - Dog
RRHLLPLLRGLARRLGSQEVREALLGCLLFILSQRHPPDAEEASRVAGQERKERLAPPK

SEQ ID NO: 40 - protein, 59 aa, Artificial Sequence
  Extracellular Myomerger - Elephant
RQHLLPLLRRLARRLGSQDMRQALLGCLLFVLSQQHPPDAGEASREALSERRGRLAPQK

SEQ ID NO: 41 - protein, 12 aa, Artificial Sequence
  alpha helix 1
LLPLLRRLARRL

SEQ ID NO: 42 - protein, 15 aa, Artificial Sequence
  alpha helix 2
QDMREALLSCLLFVL

SEQ ID NO: 43 - protein, 15 aa, Artificial Sequence
  alpha helix 2
QDMREALLGCLLFIL

(19) United States
(12) Patent Application Publication
(10) Pub. No.: US 2023/0407334 A1
(43) Pub. Date: Dec. 21, 2023

(54) PSEUDOTYPED PARTICLES, MODIFIED CELLS, RELATED COMPOSITIONS, AND RELATED METHODS

(71) Applicant: CHILDREN'S HOSPITAL MEDICAL CENTER, Cincinnati, OH (US)

(72) Inventors: Douglas MILLAY, Park Hills, KY (US); Sajedah HINDI, Cincinnati, OH (US)

(73) Assignee: CHILDREN'S HOSPITAL MEDICAL CENTER, Cincinnati, OH (US)

(21) Appl. No.: 18/250,996

(22) PCT Filed: Oct. 30, 2021
(86) PCT No.: PCT/US2021/072142

§ 371 Date: Apr. 28, 2023

Related U.S. Application Data
(60) Provisional application No. 63/108,289, filed on Oct. 31, 2020.

(51) Int. Cl. C12N 15/86; C07K 14/47; A61P 21/00

(52) U.S. Cl. CPC C12N 15/86; C07K 14/4716; A61P 21/00; C12N 2740/16043; C12N 2740/16045

(57) ABSTRACT

Some embodiments of the invention include pseudotyped particles (e.g., pseudo typed exosomes, pseudotyped VSV, and pseudo typed lentiviruses) and modified cells. Other embodiments of the invention include compositions (e.g., pharmaceutical compositions) of pseudotyped particles (e.g., pseudotyped exosomes, pseudotyped VSV, and pseudotyped lentiviruses) and modified cells. Certain embodiments of the invention include methods of making pseudotyped particles (e.g., pseudotyped exosomes, pseudotyped VSV, and pseudotyped lentiviruses) and modified cells. Other embodiments of the invention include methods of administering pseudotyped particles (e.g., pseudotyped exosomes, pseudotyped VSV, and pseudotyped lentiviruses). Further embodiments of the invention include methods of administering pseudotyped particles (e.g., pseudotyped exosomes, pseudotyped VSV, and pseudotyped lentiviruses) to treat diseases (e.g., muscular dystrophy). Additional embodiments of the invention are also discussed herein.

### CROSS-REFERENCE TO RELATED APPLICATIONS

This application claims the benefit of U.S. Provisional Application No. 63/108,289, filed Oct. 31, 2021 entitled “VIRUSES AND EXOSOMES AND RELATED METHODS” which is herein incorporated by reference in its entirety.

### GOVERNMENT RIGHTS

This invention was made with government support under AR076771 awarded by the National Institutes of Health. The government has certain rights in the invention.

### REFERENCE TO A SEQUENCE LISTING

The instant application contains a Sequence Listing that has been submitted in ASCII format via EFS-Web and is hereby incorporated by reference in its entirety. Said ASCII copy, created on Oct. 24, 2021, is named 2021_10_seq_listing_36821_04060_ST25.txt and is 93 KB in size.

### BACKGROUND

While compositions and methods for delivering polypeptides and nucleic acid molecules to cells in vitro and in vivo are known, such compositions and methods can sometimes be inefficient and/or ineffective.

Certain embodiments of the invention address one or more of the issues described above. Some embodiments of the invention include pseudotyped particles (e.g., pseudotyped exosomes, pseudotyped VSV, and pseudotyped lentiviruses) and modified cells. Other embodiments of the invention include compositions (e.g., pharmaceutical compositions) of pseudotyped particles (e.g., pseudotyped exosomes, pseudotyped VSV, and pseudotyped lentiviruses) and modified cells. Certain embodiments of the invention include methods of making pseudotyped particles (e.g., pseudotyped exosomes, pseudotyped VSV, and pseudotyped lentiviruses) and modified cells. Other embodiments of the invention include methods of administering pseudotyped particles (e.g., pseudotyped exosomes, pseudotyped VSV, and pseudotyped lentiviruses). Further embodiments of the invention include methods of administering pseudotyped particles (e.g., pseudotyped exosomes, pseudotyped VSV, and pseudotyped lentiviruses) to treat diseases (e.g., muscular dystrophy). Additional embodiments of the invention are also discussed herein.

### SUMMARY

Some embodiments of the invention include pseudotyped particles selected from the group consisting of pseudotyped exosomes and pseudotyped viruses, wherein the pseudotyped particle comprises one or more polypeptides and the one or more polypeptides comprises one or more myomaker polypeptides, one or more myomerger polypeptides, or a combination thereof. In certain embodiments, one or more myomaker polypeptides, one or more myomerger polypeptides, or a combination thereof is present on the surface of the pseudotyped particle. In other embodiments, one or more myomaker polypeptides, one or more myomerger polypeptides, or a combination thereof is present on the lipid envelope of the pseudotyped particle. In some embodiments, the lipid particle has a size of 20-500 nm, optionally 30-150 nm or 80-120 nm. In yet other embodiments, the pseudotyped particle does not comprise any nucleic acid encoding a myomaker protein and/or a myomerger protein. In still other embodiments, (a) the pseudotyped particle has one or more polypeptides on the pseudotyped particle's surface that are not found in the corresponding naturally occurring particle, (b) the pseudotyped particle has a larger amount of one or more polypeptides on the pseudotyped particle's surface than that found in the corresponding naturally occurring particle, (c) the pseudotyped particle has a larger amount of one or more polypeptides in the pseudotyped particle, by measuring the total amount of polypeptide in the pseudotyped particle, than that found in the corresponding naturally occurring particle, or (d) a combination of (a), (b), or (c). In certain embodiments, (a) the pseudotyped particle has one or more polypeptides on the pseudotyped particle's surface that are not found in the corresponding non-pseudotyped particle, (b) the pseudotyped particle has a larger amount of one or more polypeptides on the pseudotyped particle's surface than that found in the corresponding non-pseudotyped particle, (c) the pseudotyped particle has a larger amount of one or more polypeptides in the pseudotyped particle, by measuring the total amount of polypeptide in the pseudotyped particle, than that found in the corresponding non-pseudotyped particle, or (d) a combination of (a), (b), or (c). In still other embodiments, the pseudotyped particle is selected from a pseudotyped exosome, a pseudotyped VSV, and a pseudotyped lentivirus. In yet other embodiments, the pseudotyped particle is a pseudotyped exosome. In other embodiments, the pseudotyped particle is a pseudotyped exosome and the exosome is produced from an exosome producing cell that expresses or overexpresses one or more myomerger polypeptides, one or more myomaker polypeptides, or a combination thereof. In certain embodiments, the pseudotyped particle is a pseudotyped exosome and the exosome producing cell is (a) a muscle cell that overexpresses one or more myomerger polypeptides, one or more myomaker polypeptides, or a combination thereof, (b) a myoblast that overexpresses one or more myomerger polypeptides, one or more myomaker polypeptides, or a combination thereof, (c) a myotube that overexpresses one or more myomerger polypeptides, one or more myomaker polypeptides, or a combination thereof, or (d) a fibroblast that expresses one or more myomerger polypeptides, one or more myomaker polypeptides, or a combination thereof. In other embodiments, the pseudotyped particle is a pseudotyped virus. In yet other embodiments, the pseudotyped particle is a pseudotyped Vesicular Stomatitis Virus (VSV). In yet other embodiments, the pseudotyped particle is a pseudotyped VSV and the pseudotyped VSV surface comprises one or more myomerger polypeptides, one or more myomaker polypeptides, or a combination thereof. In certain embodiments, the pseudotyped particle is a pseudotyped lentivirus. In other embodiments, the pseudotyped particle is a pseudotyped lentivirus and the pseudotyped lentivirus surface comprises one or more myomerger polypeptides, one or more myomaker polypeptides, or a combination thereof. In some embodiments, at least one of the one or more myomerger polypeptides is a wt-myomerger polypeptide. In still other embodiments, at least one of the one or more myomerger polypeptides comprises at least one amino acid modification relative to a wt-myomerger polypeptide. In some embodiments, at least one of the one or more myomerger polypeptides comprises at least one amino acid modification relative to a wt-myomerger polypeptide and the at least one amino acid modification is an insertion, a deletion, or a substitution. In yet other embodiments, at least one of the one or more myomerger polypeptides is selected from the group consisting of SEQ ID NO: 17, SEQ ID NO: 18, SEQ ID NO: 19, SEQ ID NO: 20, SEQ ID NO: 21, SEQ ID NO: 22, and SEQ ID NO: 23. In certain embodiments, at least one of the one or more myomerger polypeptides is a human myomerger polypeptide. In other embodiments, at least one of the one or more myomerger polypeptides is SEQ ID NO:19. In some embodiments, at least one of the one or more myomerger polypeptides has at least an 80% sequence identity to a wt-myomerger polypeptide. In still other embodiments, at least one of the one or more myomerger polypeptides has at least a 90% sequence identity to a wt-myomerger polypeptide. In yet other embodiments, at least one of the one or more myomerger polypeptides is an extracellular wt-myomerger polypeptide. In certain embodiments, at least one of the one or more myomerger polypeptides comprises (a) amino acids 4-15 of any of SEQ ID Nos: 35-40, (b) amino acids 18-32 of any of SEQ ID Nos: 35-40, or (c) both. In other embodiments, at least one of the one or more myomerger polypeptides comprises (a) LLPLLRRLARRL (SEQ ID NO:41), (b) QDMREALLSCLLFVL (SEQ ID NO:42) or QDMREALLGCLLFIL (SEQ ID NO:43), or (c) both. In yet other embodiments, at least one of the one or more myomerger polypeptides has at least an 80% sequence identity to an extracellular wt-myomerger polypeptide. In still other embodiments, at least one of the one or more myomerger polypeptides has at least a 90% sequence identity to an extracellular wt-myomerger polypeptide. In some embodiments, at least one of the one or more myomaker polypeptides is a wt-myomaker polypeptide. In certain embodiments, at least one of the one or more myomaker polypeptides comprises at least one amino acid modification relative to a wt-myomaker polypeptide. In other embodiments, at least one of the one or more myomaker polypeptides comprises at least one amino acid modification relative to a wt-myomaker polypeptide and the at least one amino acid modification is an insertion, a deletion, or a substitution. In some embodiments, at least one of the one or more myomaker polypeptides is selected from the group consisting of SEQ ID NO: 1, SEQ ID NO: 2, SEQ ID NO: 3, SEQ ID NO: 4, SEQ ID NO: 5, and SEQ ID NO: 6. In still other embodiments, at least one of the one or more myomaker polypeptides is a human myomerger polypeptide. In other embodiments, at least one of the one or more myomaker polypeptides is SEQ ID NO:1. In some embodiments, at least one of the one or more myomaker polypeptides has at least an 80% sequence identity to a wt-myomaker polypeptide. In still other embodiments, at least one of the one or more myomaker polypeptides has at least a 90% sequence identity to a wt-myomaker polypeptide. In yet other embodiments, the pseudotyped particle comprises one or more myomaker polypeptides and one or more myomerger polypeptides. In still other embodiments, the pseudotyped particle further comprises one or more nucleic acid molecules. In still other embodiments, the pseudotyped particle does not comprise a myomerger nucleic acid molecule. In some embodiments, the pseudotyped particle does not comprise a myomaker nucleic acid molecule. In other embodiments, the pseudotyped particle does not comprise a myomerger nucleic acid molecule and does not comprise a myomaker nucleic acid molecule. In certain embodiments, the pseudotyped particle further comprises a nucleic acid encoding a gene of interest. In other embodiments, the pseudotyped particle further comprises a nucleic acid encoding a gene of interest and the nucleic acid encoding the gene of interest does not encode for a myomaker polypeptide or a myomerger polypeptide. In still other embodiments, the pseudotyped particle further comprises a nucleic acid encoding a gene of interest and the gene of interest is a therapeutic gene or a reporter gene. In certain embodiments, the pseudotyped particle further comprises a nucleic acid encoding a gene of interest and the gene of interest is a gene for delivery to a muscle cell. In yet other embodiments, the pseudotyped particle further comprises a nucleic acid encoding a gene of interest and the gene of interest is a dystrophin nucleic acid molecule. In other embodiments, the pseudotyped particle further comprises a nucleic acid encoding a gene of interest and the gene of interest encodes a dystrophin polypeptide. In yet other embodiments, the pseudotyped particle further comprises a nucleic acid encoding a gene of interest and the gene of interest encodes a microdystrophin or a minidystrophin. In still other embodiments, the pseudotyped particle further comprises a nucleic acid that can modulate gene expression. In some embodiments, the pseudotyped particle further comprises a nucleic acid that can modulate gene expression selected from a gRNA/Cas9 and an anti-sense oligonucleotide. In some embodiments, the pseudotyped particle exhibits fusogenic activity with a target cell upon binding of the myomaker polypeptide and/or a myomerger polypeptide to a myomaker polypeptide and/or myomerger polypeptide on the target cell. In other embodiments, the target cell endogenously expresses a myomaker polypeptide and/or a myomerger polypeptide, optionally wherein the target cell is a muscle cell. In certain embodiments, the target cell does not endogenously expresses a myomaker polypeptide and/or a myomerger polypeptide, optionally wherein the target cell is a non-muscle cell. In some embodiments, the target cell is a fibroblast, a mesenchymal stem cell (MSC), a hematopoietic stem cell, a blood cell, a bone marrow cell, or an adipose stem cell.

Some embodiments of the invention include methods for producing a pseudotyped lentivirus comprising contacting, in any order, (a) a composition comprising cells that express at least one myomerger polypeptide, at least one myomaker polypeptide, or a combination thereof, (b) a composition comprising one or more lentivirus production plasmids, (c) a composition comprising a nucleic acid encoding a gene of interest and/or a nucleic acid that modulates gene expression, and (d) optionally, a composition comprising one or more chemicals to increase transfection or transduction efficiency; and optionally, recovering the pseudotyped lentivirus. In certain embodiments, the compositions of (b) and/or (c) are contacted with the composition of (d), and then the (b) and/or (c) with (d) mixture is contacted with the composition of (a). In other embodiments, (i) the cells inducibly express at least one myomerger polypeptide, at least one myomaker polypeptide, or a combination thereof and (ii) an induction chemical is added to the composition of (a) prior to contacting (b) or (c) or (d), after (a) contacts (b), after (a) contacts (c), after (a) contacts (d), after (a) contacts (b) and/or (c) with (d), or a combination thereof. In yet other embodiments, (i) the cells inducibly express at least one myomerger polypeptide, at least one myomaker polypeptide, or a combination thereof and (ii) an induction chemical is added to the composition of (a) prior to contacting (b) or (c) or (d). In still other embodiments, the one or more lentivirus production plasmids comprise one or more transfer plasmids, one or more packaging plasmids, or a combination thereof. In certain embodiments, the one or more lentivirus production plasmids comprise transfer plasmid plX304-GFP and packaging plasmid psPAX2. In certain embodiments, the recovering step comprises centrifugation, filtration, ultracentrifugation, ultrafiltration, chromatography, or hydrophobic interaction chromatography, or a combination thereof.

Some embodiments of the invention include methods for producing a pseudotyped VSV comprising contacting, in any order, (a) a composition comprising cells that express at least one myomerger polypeptide, at least one myomaker polypeptide, or a combination thereof, (b) a composition comprising one or more VSV production plasmids, (c) a composition comprising a nucleic acid encoding a gene of interest and/or a nucleic acid that modulates gene expression, and (d) optionally, a composition comprising one or more chemicals to increase transfection or transduction efficiency; and optionally, recovering the pseudotyped VSV. In certain embodiments, the recovering step comprises centrifugation, filtration, ultracentrifugation, ultrafiltration, chromatography, or hydrophobic interaction chromatography, or a combination thereof.

Some embodiments of the invention include methods of producing a pseudotyped exosome comprising (a) growing exosome producing cells that express at least one myomerger polypeptide, at least one myomaker polypeptide, a polypeptide of interest, or a combination thereof, (b) optionally contacting the exosome producing cells with a nucleic acid of interest, the polypeptide of interest, or a combination thereof, and (c) placing the exosome producing cells in an exosome depleted media. In certain embodiments, the method further comprises recovering the pseudotyped exosomes. In yet other embodiments, the method further comprises recovering the pseudotyped exosomes and the recovering step comprises centrifugation, filtration, ultracentrifugation, ultrafiltration, chromatography, or hydrophobic interaction chromatography, or a combination thereof.

Some embodiments of the invention include pseudotyped lentivirus produced by any method disclosed herein. Other embodiments of the invention include pseudotyped VSV produced by any method disclosed herein. Certain embodiments of the invention include pseudotyped exosomes produced by any method disclosed herein.

Some embodiments of the invention include modified cells suitable to produce any pseudotyped particle disclosed herein. Some embodiments of the invention include modified cells comprising one or more nucleic acids encoding proteins for pseudotyped particle production and nucleic acid encoding a myomaker polypeptide and/or a myomerger polypeptide. In certain embodiments, the encoded myomaker polypeptide and/or myomerger polypeptide is transiently expressed by the modified cell. In other embodiments, the encoded myomaker polypeptide and/or myomerger polypeptide is expressed from a plasmid. In some embodiments, the encoded myomaker polypeptide and/or myomerger polypeptide is stably expressed by the modified cell. In still other embodiments, the encoded myomaker polypeptide and/or myomerger polypeptide is expressed from an endogenous locus. In yet other embodiments, the myomaker polypeptide and/or myomerger polypeptide is overexpressed. In yet other embodiments, the encoded myomaker polypeptide and/or myomerger polypeptide is inducibly expressed by the modified cell. In certain embodiments, the nucleic acid encoding a myomaker polypeptide and/or a myomerger polypeptide is linked to an inducible response element, optionally a promoter. Some embodiments of the invention include modified cells comprising a nucleic acid encoding a myomaker polypeptide and/or a myomerger polypeptide, wherein the nucleic acid encoding the myomaker polypeptide and/or the myomerger polypeptide is linked to an inducible response element, optionally a promoter. In certain embodiments, the inducible response element is a doxycycline response element. In still other embodiments, the modified cell overexpresses a myomaker polypeptide and overexpresses a myomerger polypeptide. In some embodiments, the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a BHK21 cell, a modified BHK21 cell, a HEK293t cell, a modified HEK293t cell, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In yet other embodiments, the modified cell is a modified myoblast, a modified fibroblast, a BHK21 cell, a modified BHK21 cell, a HEK293t cell, a modified HEK293t cell, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

Some embodiments of the invention include compositions comprising any pseudotyped particle disclosed herein or any modified cell disclosed herein. In other embodiments, the amount of the pseudotyped particle or the modified cell is from about 0.0001% (by weight total composition) to about 99%.

Some embodiments of the invention include pharmaceutical compositions comprising any pseudotyped particle disclosed herein or any modified cell disclosed herein. In certain embodiments, the amount of the pseudotyped particle or the modified cell is from about 0.0001% (by weight total composition) to about 50%.

Some embodiments of the invention include methods for mediating fusion of a pseudotyped particle with a target cell, the method comprising contacting the target cell with any pseudotyped particle disclosed herein. Some embodiments of the invention include methods of delivering a gene of interest to a target cell, the method comprising contacting any pseudotyped particle disclosed herein with a target cell. Some embodiments of the invention include methods of delivering a gene that modulates gene expression to a target cell, the method comprising contacting any pseudotyped particle disclosed herein with a target cell. In certain embodiments, the contacting occurs in vitro or in vivo. In other embodiments, the target cell endogenously expresses a myomaker polypeptide and/or a myomerger polypeptide and optionally the target cell is a muscle cell. In some embodiments, the target cell is a muscle cell, a myoblast, a myotube, or a mesenchymal stem cell (MSC). In still other embodiments, the target cell does not endogenously express a myomaker polypeptide and/or a myomerger polypeptide and optionally the target cell is a non-muscle cell. In some embodiments, the target cell is a non-muscle cell, a fibroblast, a mesenchymal stem cell (MSC), a hematopoietic stem cell, a blood cell, a bone marrow cell, or an adipose stem cell. In still other embodiments, the target cell under expresses dystrophin, does not express dystrophin, or expresses a defective form of dystrophin.

Some embodiments of the invention include methods for administering a pseudotyped particle to an animal comprising administering the pseudotyped particle to the animal, wherein the pseudotyped particle is any pseudotyped particle disclosed herein. In certain embodiments, the administering is part of treating a disease. Some embodiments of the invention include methods for treating a disease in an animal comprising administering any pseudotyped particle disclosed herein to the animal. In other embodiments, the pseudotyped particle comprises a nucleic acid molecule comprising a gene of interest. In still other embodiments, the gene of interest encodes a dystrophin polypeptide. In yet other embodiments, the gene of interest encodes a microdystrophin or a minidystrophin. In certain embodiments, the pseudotyped particle comprises a nucleic acid molecule that can modulate gene expression. In some embodiments, the pseudotyped particle comprises a nucleic acid molecule that can modulate gene expression selected from gRNA/Cas9 and anti-sense oligonucleotides. In still other embodiments, the pseudotyped particle is a pseudotyped exosome. In certain embodiments, the pseudotyped particle is a pseudotyped exosome and the pseudotyped exosome comprises a dystrophin polypeptide. In other embodiments, the pseudotyped particle is a pseudotyped VSV. In some embodiments, the pseudotyped particle is a pseudotyped VSV and the pseudotyped VSV comprises a nucleic acid molecule comprising a gene of interest. In still other embodiments, the pseudotyped particle is a pseudotyped VSV and the pseudotyped VSV comprises a nucleic acid molecule comprising a dystrophin nucleic acid molecule. In yet other embodiments, the pseudotyped particle is a pseudotyped VSV and the pseudotyped VSV comprises a nucleic acid molecule that can modulate gene expression. In certain embodiments, the pseudotyped particle is a pseudotyped lentivirus. In other embodiments, the pseudotyped particle is a pseudotyped lentivirus and the pseudotyped lentivirus comprises a nucleic acid molecule comprising a gene of interest. In some embodiments, the pseudotyped particle is a pseudotyped lentivirus and the pseudotyped lentivirus comprises a nucleic acid molecule comprising a dystrophin nucleic acid molecule. In still other embodiments, the pseudotyped particle is a pseudotyped lentivirus and the pseudotyped lentivirus comprises a nucleic acid molecule that can modulate gene expression. In yet other embodiments, the administering is parenteral administration, mucosal administration, intravenous administration, depot injection, subcutaneous administration, topical administration, intradermal administration, oral administration, sublingual administration, intranasal administration, or intramuscular administration. In certain embodiments, the administering is an injection or an intramuscular injection. In other embodiments, the animal is selected from mammals, primates, monkeys, macaque, rhesus macaque, or pig tail macaque, humans, canine, feline, bovine, porcine, avian, chicken, mice, rabbits, and rats. In some embodiments, the animal is a mouse, rat, or human. In still other embodiments, the disease is a muscle related disease. In yet other embodiments, the disease is a disease where the animal's cells under express dystrophin, do not express dystrophin, or express a defective form of dystrophin. In certain embodiments, the disease is myopathy, muscular dystrophy, amyotrophic lateral sclerosis (ALS or also called Lou Gehrig's disease), glycogen storage disease type II (also called Pompe disease), rhabdomyosarcoma (RMS), or sarcopenia. In some embodiments, the disease is muscular dystrophy. In some embodiments, the animal is in need of treatment of a disease.

Other embodiments of the invention are also discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the description of specific embodiments presented herein.

FIG. 1: Myomaker (Mymk) and Myomerger (Mymg) track with biological vesicles.

FIG. 2: Myomaker (Mymk) and Myomerger (Mymg) enhance exosome homing to myogenic cells.

FIG. 3: Myomaker (Mymk) and Myomerger (Mymg) can be pseudotyped on viral vesicles.

FIG. 4: Myomaker (Mymk) and Myomerger (Mymg)-pseudotyped VSVAG transduces fusing myogenic cells in vitro.

FIG. 5: Myomaker (Mymk) and Myomerger (Mymg)-pseudotyped VSVAG transduces myogenic cells in vivo.

FIG. 6: Myomaker (Mymk) and Myomerger (Mymg)-pseudotyped lentivirus is functional in vitro.

FIG. 7: Myomaker (Mymk) and Myomerger (Mymg)-pseudotyped lentivirus transduces skeletal muscle in vivo.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include pseudotyped particles (e.g., pseudotyped exosomes, pseudotyped VSV, and pseudotyped lentiviruses) and modified cells. Other embodiments of the invention include compositions (e.g., pharmaceutical compositions) of pseudotyped particles (e.g., pseudotyped exosomes, pseudotyped VSV, and pseudotyped lentiviruses) and modified cells. Certain embodiments of the invention include methods of making pseudotyped particles (e.g., pseudotyped exosomes, pseudotyped VSV, and pseudotyped lentiviruses) and modified cells. Other embodiments of the invention include methods of administering pseudotyped particles (e.g., pseudotyped exosomes, pseudotyped VSV, and pseudotyped lentiviruses). Further embodiments of the invention include methods of administering pseudotyped particles (e.g., pseudotyped exosomes, pseudotyped VSV, and pseudotyped lentiviruses) to treat diseases (e.g., muscular dystrophy). Additional embodiments of the invention are also discussed herein.

Myomaker Polypeptides and Myomaker Nucleic Acid Molecules

Some embodiments of the invention include pseudotyped particles comprising the myomaker polypeptide, the myomaker nucleic acid molecule, or both, compositions comprising the pseudotyped particles, or uses of the pseudotyped particles. In some embodiments, the myomaker polypeptide is the myomaker protein disclosed in WO 2014/210448 A1, which is herein incorporated by reference in its entirety. In other embodiments, myomaker polypeptide is the myomaker protein disclosed in Table 10A of WO 2014/210448 A1. In some embodiments, the myomaker polypeptide is the myomaker protein disclosed in WO 2018/152103 A1, which is herein incorporated by reference in its entirety. In other embodiments, myomaker polypeptide is the myomaker protein disclosed in Table 2 of WO 2018/152103 A1.

The term “myomaker polypeptide” encompasses “wt-myomaker polypeptides” (i.e., myomaker polypeptides found in nature without any purposely human-made modification) and “mutant myomaker polypeptides” (e.g., with one or more modifications made to a wt-myomaker polypeptide). Nonlimiting examples of wt-myomaker polypeptides are found in Table 10A of WO 2014/210448 A1, in Table 2 of WO 2018/152103 A1, or in Table 1A. In other embodiments, the myomaker polypeptide has at least one amino acid modification relative to a wt-myomaker polypeptide. A wt-myomaker polypeptide can, in some embodiments, be a myomaker polypeptide from any animal including but not limited to a mammal, a rat, a cat, a rabbit, a human, a cow, a chicken, a turkey, a monkey, a tree shrew, a dog, a pig, a shrew, an elephant, or an opossum. Table 1A provides nonlimiting examples of wt-myomaker polypeptides and Tables 1B and 1C provide nonlimiting examples of related nucleic acid sequences (including start and stop codons).

TABLE 1A
Myomaker

| Source | Polypeptide sequence |
|---|---|
| Human | MGTLVAKLLLPTLSSLAFLPTVSIAAKRRF |
| | HMEAMVYLFTLFFVALHHACNGPGLSVLCF |
| | MRHDILEYFSVYGTALSMWVSLMALADEDE |
| | PKRSTFVMFGVLTIAVRIYHDRWGYGVYSG |
| | PIGTAILIIAAKWLQKMKEKKGLYPDKSVY |
| | TQQIGPGLCFGALALMLRFFFEDWDYTYVH |
| | SFYHCALAMSFVLLLPKVNKKAGSPGTPAK |
| | LDCSTLCCACV (SEQ ID NO: 1) |
| Dog | MGTLAAKLLLPTLSSLAFLPTVSIAAKRRF |
| | HMEAMVYLFTMFFVALHHACNGPGLSVLCF |
| | MRHDVLEYFSVYGTALSMWVSLMALADFDE |
| | PKRSTFVMFGVLTIAVRIYHDRWGYGVYSG |
| | PIGTAVLIIATKWLQQMKEKKSLYPDKSVY |
| | TQQIGPGLCFGALALMLRFFFEDWDYTYVH |
| | SFYHCALAMSFVLLLPKVNKKAGSAGPPAK |
| | LDCSTLCCACI (SEQ ID NO: 2) |
| Pig | MGTVMAKLLLPTLSSLAFLPTVSIAAKRRF |
| | HMEAMVYLFTTFFVAFYHACHGPGLAMICF |
| | LRLDILEYFSVYGTALSMWVSLMALADFDE |
| | PKRSTFVMFGVLTIAVRIYHDRWGYGVYSG |
| | PIGTAALIIAAKWLQQMKDQRRLYPDKSVY |
| | TQQIGPGLCFGALALMLRFFFEEWDYTYVH |
| | SFYHCALAMSFVLLLPKANKKAGSAGPPAK |
| | LDCSTLCCACI (SEQ ID NO: 3) |
| Mouse | MGTVVAKLLLPTLSSLAFLPTVSIATKRRF |
| | YMEAMVYLFTMFFVAFSHACDGPGLSVLCF |
| | MRRDILEYFSIYGTALSMWVSLMALADFDE |
| | PQRSTFTMLGVLTIAVRTFHDRWGYGVYSG |
| | PIGTATLIIAVKWLKKMKEKKGLYPDKSIY |
| | TQQIGPGLCFGALALMLRFFFEEWDYTYVH |
| | SFYHCALAMSFVLLLPKVNKKAGNAGAPAK |
| | LTFSTLCCTCV (SEQ ID NO: 4) |
| Opossum | MGTLVTKLLLPTISSLAFLPTISIAAKRRF |
| | HMEAMVYLFTMFFIAIYHACDGPGLSVLCF |
| | MRYDILEYFSIYGTALSMWVSLMALAEFDE |
| | PKRSTFVMFGVLTIAVRIYQDRWGYGVYSG |
| | PIGTAVLIIATKWLQKMKEKKGLYPDKSVY |
| | TQQIGPGFCFGALALMLRFFFQEWDYTYVH |
| | SFYHCSLAMSFVLLLPKVNKKAGNAGTPAK |
| | LDCSTLCCACI (SEQ ID NO: 5) |
| Zebrafish | MGAFIAKMLLPTISSLVFVPAASVAAKRGF |
| | HMEAMVYFFTMFFTAIYHACDGPGLSILCF |
| | MKYDILEYFSVYGTAISMWVTLLALGDFDE |
| | PKRSSLTMFGVLTAAVRIYQDRLGYGIYSG |
| | PIGTAVFMITVKWLQKMKEKKGLYPDKSVY |
| | TQQVGPGCCFGALALMLRFYFEEWDYAYVH |
| | SFYHVSLAMSFILLLPKKNRYAGTGRNAAK |
| | LNCYTLCCCV (SEQ ID NO: 6) |

TABLE 1B
Myomaker

| Source | cDNA nucleic acid sequence |
|---|---|
| Human | atggggac gctggtggcc aagctgctcc tgcccaccct cagcagcctg gccttcctcc ccactgtcag |
| | catcgcggcc aagaggcggt tccacatgga ggccatggtc tacctcttca ccctgttctt |
| | cgtggcgctc caccatgcct gcaatggacc cggcttgtct gtgctgtgct tcatgcgtca cgacatcctg |
| | gagtatttca gtgtctacgg gacagccctg agcatgtggg tctcgctgat ggcactggcc |
| | gacttcgacg aacccaagag gtcaacattt gtgatgttcg gcgtcctgac cattgctgtg cggatctacc |
| | atgaccgatg gggctacggg gtgtactcgg gccccatcgg cacagccatc ctcatcatcg |
| | cggcaaagtg gctacagaag atgaaggaga agaagggcct gtacccagac aagagcgtct |
| | acacccagca gataggcccc ggcctctgct tcggggcgct ggccctgatg ctacgcttct |
| | tctttgagga ctgggactac acttatgtcc acagcttcta ccactgtgcc ctggctatgt cctttgttct |
| | gctgctgccc aaggtcaaca agaaggctgg atccccgggg accccggcca agctggactg |
| | ctccaccctg tgctgtgctt gtgtctga (SEQ ID NO: 7) |
| Dog | atgggga cgctcgcggc gaagctgctc ctgcccaccc tcagcagcct ggccttcctc cccaccgtca |
| | gcatcgccgc caagcggcgg ttccacatgg aggccatggt ctacctcttc accatgttct |
| | tcgtggcact ccaccacgcg tgcaacgggc ccgggctatc ggtgctctgc ttcatgcgcc |
| | acgacgtcct ggagtacttc agcgtctatg ggacggcact gagcatgtgg gtctcgctga |
| | tggcactggc tgacttcgac gaacccaaga ggtcgacttt tgtgatgttt ggcgtcctga ccatcgccgt |
| | gcggatctac catgaccgct ggggctacgg ggtgtactcg ggccccattg gcacggctgt |
| | cctcatcatc gccacaaagt ggctgcagca gatgaaggag aagaagagtc tgtacccgga |
| | caagagtgtc tacacccagc agataggccc tggcctctgt tttggggcac tggcccttat gctgcgcttc |
| | ttttttgagg actgggatta cacctatgtc cacagcttct accactgtgc cctggccatg tccttcgtcc |
| | tcctgctccc caaggtcaac aagaaggctg gaagcgcggg gccccctgcc aagctagact |
| | gctctaccct ttgctgtgct tgcatctga (SEQ ID NO: 8) |
| Pig | atgg ggaccgtcat ggccaaactg ctgctaccca cgctgagcag cctggccttc ctccccacgg |
| | tcagcatcgc tgccaagcgg cggttccaca tggaggccat ggtctatctc ttcaccacgt tcttcgtggc |
| | gttctaccac gcctgccacg ggccgggcct ggctatgatc tgctttctgc gccttgacat cctggagtat |
| | ttcagcgtct acggaaccgc cctgagcatg tgggtctcgc tgatggcgct ggctgacttc |
| | gacgagccca agaggtcgac tttcgtgatg tttggcgtcc tgaccatcgc cgtgcggatc |
| | taccacgacc gctggggcta cggcgtgtac tcgggcccca tcggcacggc cgccctcatc |
| | atcgcggcca agtggctgca gcagatgaag gaccaacggc gcctgtatcc agacaagagc |
| | gtgtacacac agcagatagg ccccggcctc tgcttcgggg cgctggccct catgctgcgc tttttcttcg |
| | aggagtggga ttatacctac gtccacagct tctaccactg cgccctggcc atgtccttcg tcctgctgct |
| | gcccaaggcc aacaagaagg ctggaagcgc agggccaccc gccaagctgg actgctccac |
| | cctctgctgt gcttgtatct ga (SEQ ID NO: 9) |
| Mouse | atgg ggacagttgt agccaaactg ctcctgccta ccctcagcag cctggccttc ctcccgacag |
| | tgagcatcgc taccaagagg cgtttctaca tggaggccat ggtctacctc ttcaccatgt tctttgtggc |
| | gttctcccat gcctgtgatg ggcctggttt gtctgtgctg tgcttcatgc gccgtgacat tctggagtac |
| | ttcagcatct atggaacagc cctgagcatg tgggtctccc tgatggcact ggccgacttt |
| | gatgaacccc agagatcgac cttcacaatg cttggcgtcc ttaccatcgc tgtgcggact ttcatgacc |
| | gctggggtta cggggtatac tccggtccca taggcacggc caccctcatc attgctgtaa |
| | agtggctgaa gaagatgaaa gagaagaagg gcctgtaccc cgacaagagc atctacaccc |
| | agcagatagg ccccggcctg tgctttgggg ccctggccct gatgcttcga ttcttctttg aggaatggga |
| | ttacacctac gtccacagct tctaccactg tgccctggcc atgtcctttg tcctgctgct gcccaaggtc |
| | aacaagaagg ctgggaacgc aggggccccc gccaagctga ccttctccac cctctgctgc |
| | acttgtgtct ga (SEQ ID NO: 10) |
| Opossum | atggg gactcttgtt accaagttgc ttcttcccac aatcagcagc ctcgcctttc tccccaccat |
| | cagcatcgct gctaagagga gattccacat ggaagccatg gtctacctct tcaccatgtt cttcatagca |
| | atatatcatg catgtgacgg gccaggctta tcagtgctat gcttcatgcg ctatgacata ctggagtatt |
| | tcagcatcta tgggacagca ctgagcatgt gggtgtcatt aatggcactg gcagagttcg |
| | atgaaccaaa aaggtcaacc tttgtaatgt ttggcgtgtt gactattgcc gtgaggatct accaagaccg |
| | gtggggatat ggggtatact cggggcctat tggcacagct gtccttatca ttgcaacaaa |
| | atggctgcaa aagatgaaag agaagaaggg tctgtaccct gacaagagtg tgtacaccca |
| | acagataggc cctggtttct gttttggagc gttagcactg atgctgcgtt tctttttcca ggagtgggat |
| | tacacctatg ttcacagctt ctaccactgt tcactagcca tgtcctttgt cttgctgctg cccaaggtaa |
| | acaagaaagc tgggaatgct gggacacctg ccaaattgga ctgttctaca ctctgctgtg cttgcatctg |
| | a (SEQ ID NO: 11) |
| Zebrafish | atgggag cgtttatcgc caagatgttg ctgcccacta ttagcagttt ggtgtttgtg cctgcagcca |
| | gcgtggctgc aaagaggggc ttccacatgg aggccatggt ctatttcttc acaatgttct tcaccgcgat |
| | ttaccacgca tgtgacggtc cgggcttgtc cattctctgt ttcatgaagt atgacattct ggagtacttc |
| | agcgtgtacg ggacagccat ctccatgtgg gtcacgctac tggcgcttgg ggatttcgat |
| | gagcccaaac gctcttcgct caccatgttt ggggtgttga ccgcagctgt gaggatttac |
| | caggaccgac tgggctacgg catttactcc ggccccatcg ggacagctgt ctttatgatc |
| | acagtcaaat ggttacagaa aatgaaggaa aagaaaggcc tttatccaga caaaagtgtt |
| | tacactcaac aagtgggccc agggtgctgc ttcggtgctc ttgctttgat gcttcgcttc tattttgagg |
| | agtgggacta cgcttatgtt cacagtttct accacgtgtc tctggccatg tcctttattc tgctgctgcc |
| | caagaagaac cgttatgctg gaacgggacg taacgcagcc aaactcaact gctacaccct |
| | ctgctgctgt gtatga (SEQ ID NO: 12) |

TABLE 1C
(exons in lowercase) - Myomaker

| Source | Genomic nucleic acid sequence |
|---|---|
| Human | caagtgtgagctggggagggcaggggctcagagccgggctgggcgcagcatcagacacaa |
| (+ strand) | gcacagcacagggtggagcagtccagcttggccggggtccccggggatccagccttcttg |
| | ttgaccttgggcagcagcagaacaaaggacatagccagggcacagtggtagaagctgtgg |
| | acataagtgtagtcccagtcCTGCGGGGGGCAAGCGGTCAGTCTGGGGCCTCAGCCCCCT |
| | CCCCGAGGCTCCTCCCTCTCCAAGACCCAGCAGAGCCCCTTCAGGCCCCCGCCTCTGCCA |
| | GGGCACTGGGACACCTGCAGGAAGCCTCCCCCACGGTCGCGCTCACAGTGGTTTTTCTCT |
| | CCACCTAAACCCAGAGCAGTGAGGGCCTGTGCCATCCTCCAGGCTGCACTCCTTCCTTCT |
| | TCCCCATCCCCTCTCTCTGCTGTCCTTCTCTTCCTCCATCCTTCTCTCCCTCCTACCCTC |
| | CCTCCCTCCATCTCCCCCTCTTTTCTCTCCTTATCCCTCTTCCCCTGTTCCTCCCTCCCT |
| | CCTCCACTTTCTCCCTCCTTCCTTCCCTGTCTCCTCCCCTCCCTCCCTCCCTCCTCCAGG |
| | TGTTGGGCACCTGCCCCAGGCGTCTCCCAGGCTGTGCTGCCGTCTGAGATGCCAGCTGTC |
| | TGTAGGCAGCCAGCTTTGGTCTCTGTGACCTCCAGGTCCACACAGGCCATGGTGCTGGTG |
| | GTGCTGGGGACGGCATTGCCCCCGACATAGCCCTGGGAGGGGCTAGTGAGCAGGGACTAA |
| | TACCAGACTTTGGCCTGGGGCTGTCAGAGTCCCCCCAGCGTGGGCACAGCCCTGGTATCC |
| | CAGCTGAGCAGAGCCATGCCGAGTGGGCTCTGGGGCACAGGACACCTCCCCGCTGGGCTT |
| | GGTACctcaaagaagaagcgtagcatcagggccagcgccccgaagcagaggccggggcct |
| | atctgctgggtgtagacgctcttgtctgggtacaggcccttcttctccttcatcttctgt |
| | agCTGTGAGGACAGGAGGCCACAGCAAAGCTTTTAGGTCACAGCACTGGGGAACGCCCCT |
| | CCCCAAACCAGCCCGAGAGCTGGCCCTGCACAGGCTCACCCCAGCCCTCTCCCGGCAGGA |
| | GAGGAGGCTCAGGAGCCTCCTGCCGCACCCAGCCTCAGATGGCTTCTGCTGGACAGGGCC |
| | CTTCACGGTGCGACCCAGCAGAGACCCCAGCCTGGATGGCTGGGAAGGAAGCCACTGGGC |
| | CATGTGCCCCACAAAGACCCCGCTGCCCTCCCGCCTCTTTGAGATGTAACAACGCCACCC |
| | TCGCATGTCTCCTCCTCCCTGGAGGGGAGCTCTGGGGGGACTAGACTCCATGATTGCTTA |
| | CCAAGGAAAGTACTGGAGTACTTGGGACCTGCCAGCCCAGTGTGGCCCATGGGGATGGCA |
| | CTTGTGGTGATCCCTGAGCCATGGACAAGCATCGTTTGCTTTCCTAGTTAAAGGACCTAT |
| | CTCACTCTTCATTAGACAAACTTGGCCAGCACTGCTTCTCAGGTCCCAGTGCTTAGGAAG |
| | GCTCGCGTGGGCGTTTCCACTTACAGAGGGGTTTGCATTCCGAGGAAGATGCGGGAAGTG |
| | TGGGGCCACATCCCTGGAGCCGGCCTTGTGTTTTCTAGGCCACTTCACATGGAGTCTATT |
| | TGGGATTTTCAAGGGCAGTTGTTTCCTGGAATGAGGGTGGATTTTTCTCCCTGAGCCTGG |
| | TCCCCTCTTGGGAGGGGCTGGGGAACGACAGCCTTGTTGGGGAGGAAGGAGGGAGGGTTG |
| | GGTGATGGCGGCCTCGGAGTGGGGCCAGACCCGTGGGGGTACACTCAGGAGGCTATAGAT |
| | TTCAGTGGAATCAACTGTTAGACACACAGCGTGTGGCACAAGCCCCIGGGGGTGGGGGCA |
| | GCACCCCATAACTGCACCCATTGCTGAGTGGCCTATGCAAAGAGCACAAAGAGCCTTATG |
| | CTGGGTCAGGTCAGGTTTTGCCACCCAGTGAATTATGAATTGATGCCCGGCTTTCCATTT |
| | TCTGGAATTCCATTGCCAACAAGGAATTGAGCACCTGCAGTCCTGCAGTGGCCTGAAGAC |
| | AGCTGGACCGTGTGACCCTGGGTGCGGTGGTCAAGGCTGCCAGCCCACCTCTGGCCAGCC |
| | CTGCAGTAGTAACACCAGGGAGAAGAGAGGTGCCTGCCCCAGGTCACACAGTGGGCCTGG |
| | CACTATTGAAAGGGCGCCATCACCCAACCCTCCCTCCTTCTTCCTCCCGGGCTGCCATTG |
| | CCCAACCCCTCCCAAGAGGGGACGAGGCTCAGGGAGAAAAATCCACCCTCATTCCAGGAA |
| | ACAATTGCCCTTGAAAATCCTAAATAAACTCCATACTAAATGGTCTAGAAGACAACAATT |
| | TGAGCCCCAGATGCGGGGAGGCGGGCAGCCCATCCTCGGCTCCTGTGGCTGGATCTGCAG |
| | CCTGAGGGCCTTGGCAGTCTCGTGGCTCTTGGTGGGAAACACAGCAGTGAATTCTCTTCT |
| | GGGCAATTACAGTTCAGCCCAGTTCAGACCTGGCCAAGACCAGCGGGAGGAGCAACCTTC |
| | AGGGGCAGAAGGAGGCGAGAGGCGGGTGGCCAGGACCCAGGGCCCCAGCACGCTCCTTCC |
| | TGCCACCCACCTTGGTCCAGCCCACTTATGCCCAGCGCTCCCTCTCTCCCCACCAGGTGA |
| | CTCCCAGGGGCCTCCTGGGTCAGCCCAGGATTAGTGCTGCTTCCTCAGGTTGCAGACAGA |
| | AAGCAGGTCCTCTGTCTCCTGCTCAAAAAGTCAAGTCCAGCCAGGCGTGGTGGCTCATGC |
| | CTGTAATTCCAGCACTTTGGGAGACTGAGGCAGGCAGATTACCTGAAGTCAGGAGCTCAG |
| | GACCAGCCGGGCCAACGTGGTGAAACCCCATCGCTACTAAAAATATAAAAATTACCTGGG |
| | CGTGATGGCATGCGCCTATAATCCCAGCTACTCGGGAGGCTGAGACAGGAGAATCGCTTC |
| | AACCCGGGAGGCGGAGGTTGCAGTGAGCCAAGATGGCGCCATTGCACTCCAGCCTGGGTG |
| | ACAAGAGCAAAACTCCGTCTCAAAAAAAAAAAAAAAAAAGTCAGGTTCTGGCCCCGCCAC |
| | TGCCCTGCCATGACGTCCTGTTAAGTTGCTGAGGCCTCCATGCTTTGGTTCCTTCATAGG |
| | CCAAATGGCAAATCAGTCCCATGCTCCTTGGCTGTGGGGAGGATTGGGACGGGCTTTGCA |
| | AGCTGCCCACCAGAACTCGAGCGCTCTCCCCACAGCCGTGGGCCCTCCTGCACTGAGAGC |
| | TGCCCTCTGTCTTGCTGGGTGTCCTGCGGCTCTGGCCGGGGCTGGCAGTGTGGCTGGGCT |
| | GGACCAGGCCAGGTCCTCTCTTGGCACTTGAAACTGACCCTGAGACTTCAGGTCCACTCC |
| | AAAGAGGTGAAATGCAGCACAGGGATGTTCAGGCGGTGCCTGGGCTGCTGCAGGCCTGGA |
| | GAGCAGGCTCAGGCTGAAGCCTGCTGGCTCCCCAGGTCTGGGAGACCCTTGCAAGGGTGA |
| | GCTCCCTCCTGCTCTGGGGTCCCAGGAGATGCCCCGGGTCTATTTTTCCCTAAGATCCCT |
| | CTTTAGCTTGGGCGAGTTTGAGTGGGGTTTGGTCCCTGAGCCAGGAGGGTCTTGGTAGGA |
| | CGGAGAGAGCAGGGAGCACTGAAGACCACGTGAGGGCCTTGCTGCTCTGCAAGGGGCTGT |
| | CTGTGCTAGAAGGTCTGGCCCAGGCTGCCTCACTGTCATACCACACTCTCCCTCCTGGCT |
| | AGAACCAAGCTCGAGGCTCACTCCCTCCAGGAAGTCTTCCCAGATTACCCCAGGCCATTT |
| | TCCAAGTTGATGTTGCATCTCTAAAGCAGCTGGTAGTAAGAGCGGTGATGAGAGTGATAA |
| | CAAATAGCTCTTATGTGCGGAGCACATTGGAAGCCAGGCTCCATGCCAGGACTTCAGGTG |
| | CCTGATCTCAGTGAGTCTTTGAACCACCCCATGAGACAGGCAGGGGGCTGTAATGACAAC |
| | ACCTGCTTTACAGGTACGGGCGTGGAGGTGAGACATTGGGTAACTTGGGCTCAGTCTGGA |
| | GCTGGTGAGTACAGACAAGCGTCACACACAGTCTACACAGCCGGAGCACCTCATGGCTAT |
| | TTTCTACGTGGTTTTGCTGAATTCCTGCATCCACCCATTTGCCTATGAGGGCAGGAGGTA |
| | AATGAAGATCCGAGGCAGGAGGAGTCAGACAGGGGAGAGGTGACGGGCCTCCTGGGTCCC |
| | CGTTCATCGAGGCTCGCGCAGTACGCACccactttgccgcgatgatgaggatggctgtgc |
| | cgatggggcccgagtacaccccgtagccccatcggtcatggtagatccgcacagcaatgg |
| | tcaggacgccgaacatcacaaatgttgacctcttgggttcgtcgaagtcggccagtgCTG |
| | GAGGGGCCAGGGAGACACAGGGGGAGGTGAGTGGTCTCTCTTGCTCCTCCTGGCTACCCC |
| | CCCACCCCCCAGCCCCCAGGAGGCATCCTGTAGATGCCCTCTCTCGGTGTCCCCTCAGCC |
| | AGCGAGACCCTGAGGCCCAGCCTGGTCATGGAGGGGTCTGAATTCCAGCCAGTTTGAGAG |
| | GACAGGCAGCCTGCTGCTTCCCCATGGACACAGCAGCTTGGATTGTGCTCCCAGCACCTC |
| | ATTTTAATAAACAGACCACAGCTGGTTGTGGTGGCTCAGGTCTGCAATCCCAGTGCTTTG |
| | GGAGGCAGAGGCAGGAGGATCGTCTGAGACCAGGAGTTCAAGACTAGCCTGGGCAACATA |
| | GCGGGACCCCCATCTCCACAAAAAATTCGGTGGGTGTCGTGGTGCATGCCTGTCATCCCA |
| | GCTACTTGGGAGGCTGAGGTGGGAGGATGGCTTGAGGCTGTGAGTTCGAGGCTGCAGTGA |
| | GCCGTGTTTGTGCCACTGTACTCTGGCCTGAGTGACAGAGTGAGACCCTGTGGCTAAAAA |
| | TCAATAATCACTATGCAAAGTGAATAGGATCGAATCTATCCCATAGGATCACAGGACAAA |
| | GACACTAAGATTCAAGAGAAGAAATGAAGCCCCTCACAGGCCCGGTTAGATGGCAAGGAG |
| | CCTCAGGTCATGGGGACCTTGCCACAGACAACAGTTACGTGGAAAAAAACATGGTGGGAA |
| | AGGGGGCTTATGAACAGTCCCGTCTTCCAGGCTGGATATCACCCGTGTGTGTGGATGTTT |
| | GTATGACAGTCTGGGAAGCCAACCCCCCTGAGCAGTGAACAGCGGTCCTCCCAGGGAAGG |
| | AGTGACGGGAGGGAGCCCTTTCACTTTTTCCTTTGTATGCCTCTGCTGTTGAAATGTGTC |
| | ACAACAAGCTTTTACTAAATGAGTCATTTTAAAAGGATATAAAAAATCGGCATCAGGGCA |
| | TTTAAGAGGTGCATATTCTTTTTCATAGATTAAGCACAACCCTGAAACCCAGACAAGGGA |
| | AGACATTCCTGGGGCTGGGAGTGAGTGGGGATAGAGGGCTGCAGCGGGACTGGTTTGAGG |
| | CTGGGTGTGCGGACACTGGGGAGCCGGTCCTTGTCCGCAAGGCTTGTCTGCAGGGGTTGA |
| | CCACTCACccatcagcgagacccacatgctcagggctgtcccgtagacactgaaatactc |
| | caggatgtcgtgacgcatgaagcacagcacagacaagccgggtccattgcaggcatggtg |
| | gagCTGCCAGAAAACCCACAGGTGGTCACAGCACAAAGAGGCCAGAGCTGGTCCCCGAGC |
| | CACGGCCCCCAGAGTGCCAGGTCACTTGCTGGCTGTGAGAAGTCACTTTGGCGAGTCACT |
| | TAATGACTGTGTGCCTCAGTCTCCCCGTCTGAAAAATGGGGGTACTGCCGAGCACTCCCG |
| | CAGAGGGTCCTGTGGGGATTAAGTGGCACATGCCAGCGAGGTGTTTAGGGGCTGGGGTGT |
| | GCCAAGGGTTCACTCAATGTCACCTCAGCAGAATTCGCTCATCTGCACTGGCAGGACTGG |
| | GCGGAGACTGAGTGGTCACTCAGGTGAAGCCCGCTTAGGTGGGGCGGTCTCCGGGAGGGA |
| | CCCTACACGGCTCTCCCCGGACCTTCAGCATCTGTGCTTCCTTGAAGCACACAGCTGCGT |
| | GTTCACTCGCCAATCTTTGGATGTGAGGTCAGAGCCTCTCTGGGGGCTCCTTTGCTCTTT |
| | GGGGGCTCCTGGGGCCTTCTCTTGCACAAATTACCCCTCTGATGACTGGTCTACACTGCA |
| | GCAGCGTTCTCAGGCTTGAGTGGGCATCAGAACGCCTGGGGCCTTGTTTAGACACAGGTT |
| | ACTGAGCCCTGCCTAGGGTTGCTGATTAGGGAGGGCTGGGTTGGGTAGAAAATGTGCATT |
| | TTGAACACATTCCCTGTGGCACTGCTGAGGCTGGCAGGGCCCACACTGAGAGCCGGGCTG |
| | TAGCTCCTGGTTTCTGTTGCCTTAACGTGGACGAAGATCTCTGAGACCCCCTTGCAGAAG |
| | CTGAACACAGCCCCCTAGGCTCATCCATCTCTGCCCTATACTCTCGTCGTCGCCTCCCCA |
| | ACACCCACTTTCATGGCAATTTTTAAGGCAAAAGGCTTATAGGGAGTGTTTTCAAAGCAG |
| | TCAACTACTTTTCTACGGAAAACAACTCTCTCTCCTTTTGCATTCGCATTTCATCATTTT |
| | AGGTAATATTTAATTACATGACATAATTATTTTGACAGGTTCAACTGGCACAAACAAGCT |
| | TGGGAAACAGCACGGTGGACTCTTGGTCAGCCCAGCTCAGCGGGAGGAGCAGGCGTGCTG |
| | GAAAGCAGCCCGTGTCTGGAGGCGACAGGGACAGCACAGAGGGAGCGGGGGCCCTGGGTG |
| | ATCTGGGGGGCAGGCAATTCGGGGTCAAAGTGGAGTGCTTCTACTGATGGCAATTGTACA |
| | CGGCCTAAAGTGACGGTGCACCTAGGAGGCATTAATAGGGATCCAGCATCTAAAATGAGG |
| | GAGGCGGCGGTCCTGCTTCTCTCTGTTCTTGTCAGGCTCATCCAGAAGACTATGCCGAGC |
| | TCTGTGTGGTGCACCTTTCTTGAAGTGAGACTGGGAAAGACGCGGCTAGAGGAGGGTGAC |
| | CAGCGGTGGACATGACTGTTTACCTTGGGGACAGGGAAGCTTCAGGAGGGGCCTGATCAA |
| | GGTGCTTACACCTCTGTGGGAAAGAGGAGCGAGGAAGACTCCGGCCCTAGGCTGTTCTCC |
| | TGTTCTCCTGGCTTCTTCCCATCCCCCACCCCAGCCCCATCACCTGCTGTCTGTGTGCCT |
| | CAATGTAGCACAGATGGTCATGTGTGATTAAGGCATTCACTGTGAGATTGTGATAAGGCC |
| | TGTGCCCTTGCCCTGCCAGGAGCAGGAATGGCTCTGTCTGGTCCCAGTTGCATGGACGGC |
| | TCCCAGCATAGAGTGCTTGCTGCATGTGTTCAGGGAGGGGGACGCCAGGCTCTGAGAATT |
| | CTAAAGGACAGCCAGCTCACCCTGGGGACCCAGAGCCTCTGCCACTAGGCCCTTGGCTCC |
| | TCCCAATGGTGGGAACTTAGCTCCATTCGACAGATGGGGAAAGTGAACTTCAGAGCAGCA |
| | CTGCCTGCCCAAAGAGGTGAAACAGAGCAGTGCTTGGCACCTGGCCACTTCCTCCCATCC |
| | TGCAGTGCAGGGGGCAGACCTGGCCCAGCCGGGGCACTGGTGGGGTGGGTGCGGCTGAGG |
| | GCCTGGGGGGTCAGAGCTCAGGCTCGGGGAGTCTGACTTTGCAGATGTTCCCAGTGGGGG |
| | CTCAGGTGAGTGGCTGTCGGGGGGGGGCCTCCTCTGTTGTGTGGGGACAAGCACACTGTC |
| | TCCGTGGGGTTTGCACCCATAGCAAGGTGTCCGGCACAGAGATGGAGATTGTCACGGGAG |
| | GGGCCTGATTGGAAGGGAAGGGACGCCATGCGGGTGGCAGAACTTTGGGAGGGACTGAGT |
| | GTGGCTTTGAGTTCAGAAGACGTTTGTCACAAGAGGCAGCTGCCCCTGCCACTCTGGGTG |
| | GGGCAGGGTGGGGCCTCTGAGACCAGTGCAGAGGCAGCTGCGGGGCCAGCCTAGGCCCAG |
| | GCAGGGAGGTGTGGCCTGGTGGGTGCTTGTGGTTTGCTGGGCTAGGTCTAACAGGAGCCT |
| | TGAGAACAAGACCTCAGCTTTTCTCCCTGCGCTAAGGCCATGGGACCTGCAGAGAAATCC |
| | TGGCTCTGCTCTGGGCTTCAGTCTCTCATCTGCCCAAGAGGCTTCCTAGCCCTAGCCCAG |
| | GCTGGAGTCCCAGAGGAGCGAATGCAGTGGCATTTGGGTGAGTCAGGAGCTCTGGAGAGC |
| | TTGATGGTCACAGTGACACAAGTGACTCTGTCTCTCTGGGATTTGGTTTCTTCATCTGCC |
| | AAATGGGAATCAAGATCCTAGGCTTGTGGGGAAGGTGAAAAGGCTGAATCAGACACTGTG |
| | CACAGAGCGCCTAGCCGAGTCCTCTGCCCTGGGTACTGGCGCTCGAGGTGGACTCAGAAG |
| | CTCCAGGGCATCTGGTTCCACAAAGGACCCAGCCTGTCCCAGGCCACTGTCACCCCTGGG |
| | AGTGGCACACACTGGAGGGAATGCCTCGCTCCCAGCCCACACGTGCACACTCAGCTTCTG |
| | CCATTGCGGGCAAAATTGGACTTGACCAATTCAGGATACAAGCATAACATGTGAATATAT |
| | GCTTGCAAACACACGTGTGAGCTCACGGGCCTCACCCGCTCAGGACTCCCTCTGTGCACT |
| | CACATGCACTTGGCATTCTTGCCCATAGAGGCCCTGCTGCTGGAGAAGGAGGCTGTCTGG |
| | GGAGAGGAGGTGGAGTTTTCACAGGTTGGGCCCAGCACTGCCCCAAGAAGGAGGCTAGTG |
| | GGACGCTTGCCTCCCCAGAGCAGGTGTCATGCTGGGGATTGGGCTGTCAGTGAAGGAGGG |
| | GTGTGATGGAAGGTGAGCAAGGAAGGCTTCGGGAGAGCAAGAGGTGGGGCACCACTTGTG |
| | GGAGTCCAGGAGTGAGGGCATGTTAGTGGAGAAAGTCGGAAAGACCCAGAGGCAAGAAGG |
| | CAGGGGGTACCGAGACATATAAATGATGGCTGAATGGCGAGATGGTAATAGACGAATAGA |
| | TCACAGGTAGATGGATGCGTAGATAGAGAGATAGATGGAGAGAGAGAGAGAGAGAGAGAG |
| | AGAGAGAGAGAGAGACAAGCTGGAGAAGGTGGATAGCTAAAGCCAGAGAGACACATGGAG |
| | AGTCAGGGGACTAAAACCAGGGAGGGTGGGACCAAGAGCTTTAGAGAGAGTGAATTCCAT |
| | GGGGATCGAGTTCCAGAAATCAAAAGAGAACCAGACAGAGAGAGAAAGGAAAAAAAGAGA |
| | AACAGAGAAAACTAGACACAGAAAACCAATACGAGAAACACAGAGTGAAAGAGACCCAGA |
| | AAGAGAGAGAGAGAAGACAGGGGAGACAGGGGTCCCAGAAACAGCGACCTCAGAAACAAG |
| | GACAGATGGGGTTCTGGGCGCTCCACTGAAAGCCGGATAAGATCACCCAATGACAGGTAC |
| | CAGGAAACAGAGAGCAGGAGAGAGCCAGAGAGAGAGCAAGCGGAGACAGTCAGCCAGCCA |
| | GACACATAAATAGAAAGAGAAAGACGGACCCACAGAGAGAGAAGTAGGCCCCAGAAGAGG |
| | GAGAGACCAGCAGGCCCTCCTGAACCAGAGCAGCTCCAGGATTCTGGAATCAGACTCACT |
| | CACCCAGGCCTTCACACTCCCTGAACCCTGCAGACCCCTTCCCAGGCCTGGCTTGCCCCA |
| | CTCATCTCTGCTCCATCGTGGCCTATGGGTAGAGCTCGAAGAGAGGTGGGGaGGGGAGGT |
| | GGCCCCATGGGCAGCCGTGGGGGCTTTGATTAGCAGCTGAGAAAAGGGGCACGCTGGAAG |
| | GGTTTATCCTCAACTCAATGGCCCTGCTTCACCCCAGGCTTGGTCTCACACAGGCAGTGA |
| | TCCCAGAGCAACTTCCTGGCACAGATGGGAAAACTGAGGTCCAGATAGGGGAAGGGACTC |
| | CCCTAGTCCTCTCTCTTCAGTCTCCAGACCCCACCTGGGCCTGCTGTTTCATTTTCAAAT |
| | CACTTCTGCTCATCACCCAATACAAGAACGCTGTGGACAGAGAGCCTCTCCTCTACCTCC |
| | AGGATGGGGCCTGTGTGGGACTTCCTCCCAGCCCCCAGACTCACcgccacgaagaacagg |
| | gtgaagaggtagaccatggcctccatgtggaaccgcctcttggccgcgatgctgacagtg |
| | gggaggaaggccaggctgctgagggtgggcaggagcagcttggccaccagcgtccccatg |
| | ggccaggaggaaagcactggctggggggggagggtgctggtgtcccaggtccccagcac |
| | aggagcacgaagtgggaaggccagctccctttgggcagggc |
| | (SEQ ID NO: 13) |
| Human | gccctgcccaaagggagctggccttcccacttcgtgctcctgtgctggggacctgggaca |
| (− strand, | ccagcaccctccccaccccagccagtgctttcctcctggcccatggggacgctggtggcc |
| reverse | aagctgctcctgcccaccctcagcagcctggccttcctccccactgtcagcatcgcggcc |
| complement) | aagaggcggttccacatggaggccatggtctacctcttcaccctgttcttcgtggcgGTG |
| - start codon | AGTCTGGGGGCTGGGAGGAAGTCCCACACAGGCCCCATCCTGGAGGTAGAGGAGAGGCTC |
| is bold & | TCTGTCCACAGCGTTCTTGTATTGGGTGATGAGCAGAAGTGATTTGAAAATGAAACAGCA |
| underlined; | GGCCCAGGTGGGGTCTGGAGACTGAAGAGAGAGGACTAGGGGAGTCCCTTCCCCTATCTG |
| stop codon is | GACCTCAGTTTTCCCATCTGTGCCAGGAAGTTGCTCTGGGATCACTGCCTGTGTGAGACC |
| bold and | AAGCCTGGGGTGAAGCAGGGCCATTGAGTTGAGGATAAACCCTTCCAGCGTGCCCCTTTT |
| italicized | CTCAGCTGCTAATCAAAGCCCCCACGGCTGCCCATGGGGCCACCTCCCCTCCCCACCTCT |
| | CTTCGAGCTCTACCCATAGGCCACGATGGAGCAGAGATGAGTGGGGCAAGCCAGGCCTGG |
| | GAAGGGGTCTGCAGGGTTCAGGGAGTGTGAAGGCCTGGGTGAGTGAGTCTGATTCCAGAA |
| | TCCTGGAGCTGCTCTGGTTCAGGAGGGCCTGCTGGTCTCTCCCTCTTCTGGGGCCTACTT |
| | CTCTCTCTGTGGGTCCGTCTTTCTCTTTCTATTTATGTGTCTGGCTGGCTGACTGTCTCC |
| | GCTTGCTCTCTCTCTGGCTCTCTCCTGCTCTCTGTTTCCTGGTACCTGTCATTGGGTGAT |
| | CTTATCCGGCTTTCAGTGGAGCGCCCAGAACCCCATCTGTCCTTGTTTCTGAGGTCGCTG |
| | TTTCTGGGACCCCTGTCTCCCCTGTCTTCTCTCTCTCTCTTTCTGGGTCTCTTTCACTCT |
| | GTGTTTCTCGTATTGGTTTTCTGTGTCTAGTTTTCTCTGTTTCTCTTTTTTTCCTTTCTC |
| | TCTCTGTCTGGTTCTCTTTTGATTTCTGGAACTCGATCCCCATGGAATTCACTCTCTCTA |
| | AAGCTCTTGGTCCCACCCTCCCTGGTTTTAGTCCCCTGACTCTCCATGTGTCTCTCTGGC |
| | TTTAGCTATCCACCTTCTCCAGCTTGTCTCTCTCTCTCTCTCTCTCTCTCTCTCTCTCTC |
| | TCTCCATCTATCTCTCTATCTACGCATCCATCTACCTGTGATCTATTCGTCTATTACCAT |
| | CTCGCCATTCAGCCATCATTTATATGTCTCGGTACCCCCTGCCTTCTTGCCTCTGGGTCT |
| | TTCCGACTTTCTCCACTAACATGCCCTCACTCCTGGACTCCCACAAGTGGTGCCCCACCT |
| | CTTGCTCTCCCGAAGCCTTCCTTGCTCACCTTCCATCACACCCCTCCTTCACTGACAGCC |
| | CAATCCCCAGCATGACACCTGCTCTGGGGAGGCAAGCGTCCCACTAGCCTCCTTCTTGGG |
| | GCAGTGCTGGGCCCAACCTGTGAAAACTCCACCTCCTCTCCCCAGACAGCCTCCTTCTCC |
| | AGCAGCAGGGCCTCTATGGGCAAGAATGCCAAGTGCATGTGAGTGCACAGAGGGAGTCCT |
| | GAGCGGGTGAGGCCCGTGAGCTCACACGTGTGTTTGCAAGCATATATTCACATGTTATGC |
| | TTGTATCCTGAATTGGTCAAGTCCAATTTTGCCCGCAATGGCAGAAGCTGAGTGTGCACG |
| | TGTGGGCTGGGAGCGAGGCATTCCCTCCAGTGTGTGCCACTCCCAGGGGTGACAGTGGCC |
| | TGGGACAGGCTGGGTCCTTTGTGGAACCAGATGCCCTGGAGCTTCTGAGTCCACCTCGAG |
| | CGCCAGTACCCAGGGCAGAGGACTCGGCTAGGCGCTCTGTGCACAGTGTCTGATTCAGCC |
| | TTTTCACCTTCCCCACAAGCCTAGGATCTTGATTCCCATTTGGCAGATGAAGAAACCAAA |
| | TCCCAGAGAGACAGAGTCACTTGTGTCACTGTGACCATCAAGCTCTCCAGAGCTCCTGAC |
| | TCACCCAAATGCCACTGCATTCGCTCCTCTGGGACTCCAGCCTGGGCTAGGGCTAGGAAG |
| | CCTCTTGGGCAGATGAGAGACTGAAGCCCAGAGCAGAGCCAGGATTTCTCTGCAGGTCCC |
| | ATGGCCTTAGCGCAGGGAGAAAAGCTGAGGTCTTGTTCTCAAGGCTCCTGTTAGACCTAG |
| | CCCAGCAAACCACAAGCACCCACCAGGCCACACCTCCCTGCCTGGGCCTAGGCTGGCCCC |
| | GCAGCTGCCTCTGCACTGGTCTCAGAGGCCCCACCCTGCCCCACCCAGAGTGGCAGGGGC |
| | AGCTGCCTCTTGTGACAAACGTCTTCTGAACTCAAAGCCACACTCAGTCCCTCCCAAAGT |
| | TCTGCCACCCGCATGGCGTCCCTTCCCTTCCAATCAGGCCCCTCCCGTGACAATCTCCAT |
| | CTCTGTGCCGGACACCTTGCTATGGGTGCAAACCCCACGGAGACAGTGTGCTTGTCCCCA |
| | CACAACAGAGGAGGCCCCCCCCCGACAGCCACTCACCTGAGCCCCCACTGGGAACATCTG |
| | CAAAGTCAGACTCCCCGAGCCTGAGCTCTGACCCCCCAGGCCCTCAGCCGCACCCACCCC |
| | ACCAGTGCCCCGGCTGGGCCAGGTCTGCCCCCTGCACTGCAGGATGGGAGGAAGTGGCCA |
| | GGTGCCAAGCACTGCTCTGTTTCACCTCTTTGGGCAGGCAGTGCTGCTCTGAAGTTCACT |
| | TTCCCCATCTGTCGAATGGAGCTAAGTTCCCACCATTGGGAGGAGCCAAGGGCCTAGTGG |
| | CAGAGGCTCTGGGTCCCCAGGGTGAGCTGGCTGTCCTTTAGAATTCTCAGAGCCTGGCGT |
| | CCCCCTCCCTGAACACATGCAGCAAGCACTCTATGCTGGGAGCCGTCCATGCAACTGGGA |
| | CCAGACAGAGCCATTCCTGCTCCTGGCAGGGCAAGGGCACAGGCCTTATCACAATCTCAC |
| | AGTGAATGCCTTAATCACACATGACCATCTGTGCTACATTGAGGCACACAGACAGCAGGT |
| | GATGGGGCTGGGGTGGGGGATGGGAAGAAGCCAGGAGAACAGGAGAACAGCCTAGGGCCG |
| | GAGTCTTCCTCGCTCCTCTTTCCCACAGAGGTGTAAGCACCTTGATCAGGCCCCTCCTGA |
| | AGCTTCCCTGTCCCCAAGGTAAACAGTCATGTCCACCGCTGGTCACCCTCCTCTAGCCGC |
| | GTCTTTCCCAGTCTCACTTCAAGAAAGGTGCACCACACAGAGCTCGGCATAGTCTTCTGG |
| | ATGAGCCTGACAAGAACAGAGAGAAGCAGGACCGCCGCCTCCCTCATTTTAGATGCTGGA |
| | TCCCTATTAATGCCTCCTAGGTGCACCGTCACTTTAGGCCGTGTACAATTGCCATCAGTA |
| | GAAGCACTCCACTTTGACCCCGAATTGCCTGCCCCCCAGATCACCCAGGGCCCCCGCTCC |
| | CTCTGTGCTGTCCCTGTCGCCTCCAGACACGGGCTGCTTTCCAGCACGCCTGCTCCTCCC |
| | GCTGAGCTGGGCTGACCAAGAGTCCACCGTGCTGTTTCCCAAGCTTGTTTGTGCCAGTTG |
| | AACCTGTCAAAATAATTATGTCATGTAATTAAATATTACCTAAAATGATGAAATGCGAAT |
| | GCAAAAGGAGAGAGAGTTGTTTTCCGTAGAAAAGTAGTTGACTGCTTTGAAAACACTCCC |
| | TATAAGCCTTTTGCCTTAAAAATTGCCATGAAAGTGGGTGTTGGGGAGGCGACGACGAGA |
| | GTATAGGGCAGAGATGGATGAGCCTAGGGGGCTGTGTTCAGCTTCTGCAAGGGGGTCTCA |
| | GAGATCTTCGTCCACGTTAAGGCAACAGAAACCAGGAGCTACAGCCCGGCTCTCAGTGTG |
| | GGCCCTGCCAGCCTCAGCAGTGCCACAGGGAATGTGTTCAAAATGCACATTTTCTACCCA |
| | ACCCAGCCCTCCCTAATCAGCAACCCTAGGCAGGGCTCAGTAACCTGTGTCTAAACAAGG |
| | CCCCAGGCGTTCTGATGCCCACTCAAGCCTGAGAACGCTGCTGCAGTGTAGACCAGTCAT |
| | CAGAGGGGTAATTTGTGCAAGAGAAGGCCCCAGGAGCCCCCAAAGAGCAAAGGAGCCCCC |
| | AGAGAGGCTCTGACCTCACATCCAAAGATTGGCGAGTGAACACGCAGCTGTGTGCTTCAA |
| | GGAAGCACAGATGCTGAAGGTCCGGGGAGAGCCGTGTAGGGTCCCTCCCGGAGACCGCCC |
| | CACCTAAGCGGGCTTCACCTGAGTGACCACTCAGTCTCCGCCCAGTCCTGCCAGTGCAGA |
| | TGAGCGAATTCTGCTGAGGTGACATTGAGTGAACCCTTGGCACACCCCAGCCCCTAAACA |
| | CCTCGCTGGCATGTGCCACTTAATCCCCACAGGACCCTCTGCGGGAGTGCTCGGCAGTAC |
| | CCCCATTTTTCAGACGGGGAGACTGAGGCACACAGTCATTAAGTGACTCGCCAAAGTGAC |
| | TTCTCACAGCCAGCAAGTGACCTGGCACTCTGGGGGCCGTGGCTCGGGGACCAGCTCTGG |
| | CCTCTTTGTGCTGTGACCACCTGTGGGTTTTCTGGCAGctccaccatgcctgcaatggac |
| | ccggcttgtctgtgctgtgcttcatgcgtcacgacatcctggagtatttcagtgtctacg |
| | ggacagccctgagcatgtgggtctcgctgatggGTGAGTGGTCAACCCCTGCAGACAAGC |
| | CTTGCGGACAAGGACCGGCTCCCCAGTGTCCGCACACCCAGCCTCAAACCAGTCCCGCTG |
| | CAGCCCTCTATCCCCACTCACTCCCAGCCCCAGGAATGTCTTCCCTTGTCTGGGTTTCAG |
| | GGTTGTGCTTAATCTATGAAAAAGAATATGCACCTCTTAAATGCCCTGATGCCGATTTTT |
| | TATATCCTTTTAAAATGACTCATTTAGTAAAAGCTTGTTGTGACACATTTCAACAGCAGA |
| | GGCATACAAAGGAAAAAGTGAAAGGGCTCCCTCCCGTCACTCCTTCCCTGGGAGGACCGC |
| | TGTTCACTGCTCAGGGGGGTTGGCTTCCCAGACTGTCATACAAACATCCACACACACGGG |
| | TGATATCCAGCCTGGAAGACGGGACTGTTCATAAGCCCCCTTTCCCACCATGTTTTTTTC |
| | CACGTAACTGTTGTCTGTGGCAAGGTCCCCATGACCTGAGGCTCCTTGCCATCTAACCGG |
| | GCCTGTGAGGGGCTTCATTTCTTCTCTTGAATCTTAGTGTCTTTGTCCTGTGATCCTATG |
| | GGATAGATTCGATCCTATTCACTTTGCATAGTGATTATTGATTTTTAGCCACAGGGTCTC |
| | ACTCTGTCACTCAGGCCAGAGTACAGTGGCACAAACACGGCTCACTGCAGCCTCGAACTC |
| | ACAGCCTCAAGCCATCCTCCCACCTCAGCCTCCCAAGTAGCTGGGATGACAGGCATGCAC |
| | CACGACACCCACCGAATTTTTTGTGGAGATGGGGGTCCCGCTATGTTGCCCAGGCTAGTC |
| | TTGAACTCCTGGTCTCAGACGATCCTCCTGCCTCTGCCTCCCAAAGCACTGGGATTGCAG |
| | ACCTGAGCCACCACAACCAGCTGTGGTCTGTTTATTAAAATGAGGTGCTGGGAGCACAAT |
| | CCAAGCTGCTGTGTCCATGGGGAAGCAGCAGGCTGCCTGTCCTCTCAAACTGGCTGGAAT |
| | TCAGACCCCTCCATGACCAGGCTGGGCCTCAGGGTCTCGCTGGCTGAGGGGACACCGAGA |
| | GAGGGCATCTACAGGATGCCTCCTGGGGGCTGGGGGGTGGGGGGGTAGCCAGGAGGAGCA |
| | AGAGAGACCACTCACCTCCCCCTGTGTCTCCCTGGCCCCTCCAGcactggccgacttcga |
| | cgaacccaagaggtcaacatttgtgatgttcggcgtcctgaccattgctgtgcggatcta |
| | ccatgaccgatggggctacggggtgtactcgggccccatcggcacagccatcctcatcat |
| | cgcggcaaagtggGTGCGTACTGCGCGAGCCTCGATGAACGGGGACCCAGGAGGCCCGTC |
| | ACCTCTCCCCTGTCTGACTCCTCCTGCCTCGGATCTTCATTTACCTCCTGCCCTCATAGG |
| | CAAATGGGTGGATGCAGGAATTCAGCAAAACCACGTAGAAAATAGCCATGAGGTGCTCCG |
| | GCTGTGTAGACTGTGTGTGACGCTTGTCTGTACTCACCAGCTCCAGACTGAGCCCAAGTT |
| | ACCCAATGTCTCACCTCCACGCCCGTACCTGTAAAGCAGGTGTTGTCATTACAGCCCCCT |
| | GCCTGTCTCATGGGGtGGTTCAAAGACTCACTGAGATCAGGCACCTGAAGTCCTGGCATG |
| | GAGCCTGGCTTCCAATGTGCTCCGCACATAAGAGCTATTTGTTATCACTCTCATCACCGC |
| | TCTTACTACCAGCTGCTTTAGAGATGCAACATCAACTTGGAAAATGGCCTGGGGTAATCT |
| | GGGAAGACTTCCTGGAGGGAGTGAGCCTCGAGCTTGGTTCTAGCCAGGAGGGAGAGTGTG |
| | GTATGACAGTGAGGCAGCCTGGGCCAGACCTTCTAGCACAGACAGCCCCTTGCAGAGCAG |
| | CAAGGCCCTCACGTGGTCTTCAGTGCTCCCTGCTCTCTCCGTCCTACCAAGACCCTCCTG |
| | GCTCAGGGACCAAACCCCACTCAAACTCGCCCAAGCTAAAGAGGGATCTTAGGGAAAAAT |
| | AGACCCGGGGCATCTCCTGGGACCCCAGAGCAGGAGGGAGCTCACCCTTGCAAGGGTCTC |
| | CCAGACCTGGGGAGCCAGCAGGCTTCAGCCTGAGCCTGCTCTCCAGGCCTGCAGCAGCCC |
| | AGGCACCGCCTGAACATCCCTGTGCTGCATTTCACCTCTTTGGAGTGGACCTGAAGTCTC |
| | AGGGTCAGTTTCAAGTGCCAAGAGAGGACCTGGCCTGGTCCAGCCCAGCCACACTGCCAG |
| | CCCCGGCCAGAGCCGCAGGACACCCAGCAAGACAGAGGGCAGCTCTCAGTGCAGGAGGGC |
| | CCACGGCTGTGGGGAGAGCGCTCGAGTTCTGGTGGGCAGCTTGCAAAGCCCGTCCCAATC |
| | CTCCCCACAGCCAAGGAGCATGGGACTGATTTGCCATTTGGCCTATGAAGGAACCAAAGC |
| | ATGGAGGCCTCAGCAACTTAACAGGACGTCATGGCAGGGCAGTGGCGGGGCCAGAACCTG |
| | ACTTTTTTTTTTTTTTTTTTGAGACGGAGTTTTGCTCTTGTCACCCAGGCTGGAGTGCAA |
| | TGGCGCCATCTTGGCTCACTGCAACCTCCGCCTCCCGGGTTGAAGCGATTCTCCTGTCTC |
| | AGCCTCCCGAGTAGCTGGGATTATAGGCGCATGCCATCACGCCCAGGTAATTTTTATATT |
| | TTTAGTAGCGATGGGGTTTCACCACGTTGGCCCGGCTGGTCCTGAGCTCCTGACTTCAGG |
| | TAATCTGCCTGCCTCAGTCTCCCAAAGTGCTGGAATTACAGGCATGAGCCACCACGCCTG |
| | GCTGGACTTGACTTTTTGAGCAGGAGACAGAGGACCTGCTTTCTGTCTGCAACCTGAGGA |
| | AGCAGCACTAATCCTGGGCTGACCCAGGAGGCCCCTGGGAGTCACCTGGTGGGGAGAGAG |
| | GGAGCGCTGGGCATAAGTGGGCTGGACCAAGGTGGGTGGCAGGAAGGAGCGTGCTGGGGC |
| | CCTGGGTCCTGGCCACCCGCCTCTCGCCTCCTTCTGCCCCTGAAGGTTGCTCCTCCCGCT |
| | GGTCTTGGCCAGGTCTGAACTGGGCTGAACTGTAATTGCCCAGAAGAGAATTCACTGCTG |
| | TGTTTCCCACCAAGAGCCACGAGACTGCCAAGGCCCTCAGGCTGCAGATCCAGCCACAGG |
| | AGCCGAGGATGGGCTGCCCGCCTCCCCGCATCTGGGGCTCAAATTGTTGTCTTCTAGACC |
| | ATTTAGTATGGAGTTTATTTAGGATTTTCAAGGGCAATTGTTTCCTGGAATGAGGGTGGA |
| | TTTTTCTCCCTGAGCCTCGTCCCCTCTTGGGAGGGGTTGGGCAATGGCAGCCCGGGAGGA |
| | AGAAGGAGGGAGGGTTGGGTGATGGCGCCCTTTCAATAGTGCCAGGCCCACTGTGTGACC |
| | TGGGGCAGGCACCTCTCTTCTCCCTGGTGTTACTACTGCAGGGCTGGCCAGAGGTGGGCT |
| | GGCAGCCTTGACCACCGCACCCAGGGTCACACGGTCCAGCTGTCTTCAGGCCACTGCAGG |
| | ACTGCAGGTGCTCAATTCCTTGTTGGCAATGGAATTCCAGAAAATGGAAAGCCGGGCATC |
| | AATTCATAATTCACTGGGTGGCAAAACCTGACCTGACCCAGCATAAGGCTCTTTGTGCTC |
| | TTTGCATAGGCCACTCAGCAATGGGTGCAGTTATGGGGTGCTGCCCCCACCCCCAGGGGC |
| | TTGTGCCACACGCTGTGTGTCTAACAGTTGATTCCACTGAAATCTATAGCCTCCTGAGTG |
| | TACCCCCACGGGTCTGGCCCCACTCCGAGGCCGCCATCACCCAACCCTCCCTCCTTCCTC |
| | CCCAACAAGGCTGTCGTTCCCCAGCCCCTCCCAAGAGGGGACCAGGCTCAGGGAGAAAAA |
| | TCCACCCTCATTCCAGGAAACAACTGCCCTTGAAAATCCCAAATAGACTCCATGTGAAGT |
| | GGCCTAGAAAACACAAGGCCGGCTCCAGGGATGTGGCCCCACACTTCCCGCATCTTCCTC |
| | GGAATGCAAACCCCTCTGTAAGTGGAAACGCCCACGCGAGCCTTCCTAAGCACTGGGACC |
| | TGAGAAGCAGTGCTGGCCAAGTTTGTCTAATGAAGAGTGAGATAGGTCCTTTAACTAGGA |
| | AAGCAAACGATGCTTGTCCATGGCTCAGGGATCACCACAAGTGCCATCCCCATGGGCCAC |
| | ACTGGGCTGGCAGGTCCCAAGTACTCCAGTACTTTCCTTGGTAAGCAATCATGGAGTCTA |
| | GTCCCCCCAGAGCTCCCCTCCAGGGAGGAGGAGACATGCGAGGGTGGCGTTGTTACATCT |
| | CAAAGAGGCGGGAGGGCAGCGGGGTCTTTGTGGGGCACATGGCCCAGTGGCTTCCTTCCC |
| | AGCCATCCAGGCTGGGGTCTCTGCTGGGTCGCACCGTGAAGGGCCCTGTCCAGCAGAAGC |
| | CATCTGAGGCTGGGTGCGGCAGGAGGCTCCTGAGCCTCCTCTCCTGCCGGGAGAGGGCTG |
| | GGGTGAGCCTGTGCAGGGCCAGCTCTCGGGCTGGTTTGGGGAGGGGCGTTCCCCAGTGCT |
| | GTGACCTAAAAGCTTTGCTGTGGCCTCCTGTCCTCACAGctacagaagatgaaggagaag |
| | aagggcctgtacccagacaagagcgtctacacccagcagataggccccggcctctgcttc |
| | ggggcgctggccctgatgctacgcttcttctttgagGTACCAAGCCCAGCGGGGAGGTGT |
| | CCTGTGCCCCAGAGCCCACTCGGCATGGCTCTGCTCAGCTGGGATACCAGGGCTGTGCCC |
| | ACGCTGGGGGGACTCTGACAGCCCCAGGCCAAAGTCTGGTATTAGTCCCTGCTCACTAGC |
| | CCCTCCCAGGGCTATGTCGGGGGCAATGCCGTCCCCAGCACCACCAGCACCATGGCCTGT |
| | GTGGACCTGGAGGTCACAGAGACCAAAGCTGGCTGCCTACAGACAGCTGGCATCTCAGAC |
| | GGCAGCACAGCCTGGGAGACGCCTGGGGCAGGTGCCCAACACCTGGAGGAGGGAGGGAGG |
| | GAGGGGAGGAGACAGGGAAGGAAGGAGGGAGAAAGTGGAGGAGGGAGGGAGGAACAGGGG |
| | AAGAGGGATAAGGAGAGAAAAGAGGGGGAGATGGAGGGAGGGAGGGTAGGAGGGAGAGAA |
| | GGATGGAGGAAGAGAAGGACAGCAGAGAGAGGGGATGGGGAAGAAGGAAGGAGTGCAGCC |
| | TGGAGGATGGCACAGGCCCTCACTGCTCTGGGTTTAGGTGGAGAGAAAAACCACTGTGAG |
| | CGCGACCGTGGGGGAGGCTTCCTGCAGGTGTCCCAGTGCCCTGGCAGAGGCGGGGGCCTG |
| | AAGGGGCTCTGCTGGGTCTTGGAGAGGGAGGAGCCTCGGGGAGGGGGCTGAGGCCCCAGA |
| | CTGACCGCTTGCCCCCCGCAGgactgggactacacttatgtccacagcttctaccactgt |
| | gccctggctatgtcctttgttctgctgctgcccaaggtcaacaagaaggctggatccccg |
| | gggaccccggccaagctggactgctccaccctgtgctgtgcttgtgtc [symbol] tgctgcgcc |
| | cagcccggctctgagcccctgccctccccagctcacacttg |
| | (SEQ ID NO: 14) |
| Mouse | ttccaggaactagaatgtatgttaggcgaagctaatgactagtggctgatcaagagttta |
| (+ strand) | ctgtgaatggcttgatcgaaaacctgcagaagggatgggactcaggcaggggtatgcaag |
| | gttcgctggctccagcttcctaagtggagagctttcagagcctgggcaggggttaaaagg |
| | gcaatcccagtttcctagggaaagcagacgattctgacaggcaggacctgggaaatagat |
| | aaccctgcatgctgctgggtatttactggtctagggttctctgccaggcacacctatggt |
| | tgtgaggccttgggggataaagttcttttttttcctgaacagagtgaagcaactggtgaa |
| | cacagaaccagtgggtccctaagcagcactcagcagaatgcagcaggcctgctggtctct |
| | tggggtgtagagaagaccatttctcatgtacaggccgcataacaaagtataggaagtacc |
| | ttgggagagacagcaggactgccaggcaggaaggcaggggcctggtgtgtgtgtgtgtgt |
| | gggggggtatagtcagacacaagtgcagcagagggtggagaaggtcagcttggcgggggc |
| | ccctgcgttcccagccttcttgttgaccttgggcagcagcaggacaaaggacatggccag |
| | ggcacagtggtagaagctgtggacgtaggtgtaatcccattcCTGTGGAGGAGAATGAGT |
| | CAGTCTGGGCCTCCATCCCTTCCCTAAACCAAGTCCTAGCCATTTGGTGCCTCTGTCAGC |
| | CAGCCCACCCTGAGAAGGTGGCAGAAAGGCTTGCTGCCTTCCTCTGTTCCATGCCTCCTG |
| | GGTGCTGGGCACCAGCTCCTGGTTCCTTCCAGGACATGCGTGCATCTTGGGTGCAGGCTT |
| | CCTAAAGTCAGGGCCTGACTTGTCCACTCAGGCAGTGAGGCTAGTACACTGGGGATGGTG |
| | AGTACCATCCTCAAGAGGACAGAATTTACAACTTGGAGCCTCCATATGTGGCTGTTAGTT |
| | AACTATTTCCAGAGGCTCTTGCTCCCCTTCCCCATAGGCCAGGTACctcaaagaagaatc |
| | gaagcatcagggccagggccccaaagcacaggccggggcctatctgctgggtgtagatgc |
| | tcttgtcggggtacaggcccttcttctctttcatcttcttcagCTGCAGGCACAAGGTGG |
| | GGACATCAAAGTTCTTGGGGTGCAGCACAGGAAGGGACCCCTCCATGAACTGTAGAAGAG |
| | CCCTACCCCCATTCCTCTGTATGCCTGACTGATGGGACTCTCTGGGCCAATTTCCCCTGG |
| | GTCCTCTACTGCCCGCATCTGGTGGGCTTTGGCACTTCAGTGGCAGACGTGATCAGTTTT |
| | CCCAGCTAAGGGGTTTTCCTCTGTTAACCTTGGTTTCATAGGCCCTGTGTGTTCAAGCTT |
| | GGTAAGATGGAGTGTTACATGGAATAGATGGGAGTCCCATGGTTCCTCACTGGAATGCAC |
| | ATCCTTGGGGCCCAAAGGTATTTTAGGTATTCAAGATTGTTCAGGTTTCAGTGGGGAAGA |
| | TCATTATAAATACCACTGTCAGGTGTGCACAGAGGGCACAGGACAGCAGCCCTGACTGAG |
| | TGATGTGCACAGTGGGCACAGGACAGCGGCCCTAATTGCACACCTCACTAAATACATTAT |
| | ATGTACAAATGCTGTCAATGGCCTCGTGCAAATCAGGGCAAGCTTTGTCACTCTGAGTGA |
| | TGATATGTTGCTGTTTCCAAGTGTTCTAAAACTTGCCATTAGTAACAGGAGTGGAGGTCC |
| | CAGTGAGCAGTGCCAGTGACATGGGCACCGCCTATTAGCCTGAGTGTAGGCCGTATGACC |
| | ATCAATCACACAGTTCTAACACTGGGGCCCCAGAGAGGAGAAGAATATTGAAGATCACCC |
| | ATGGGCCCTGTCTTGCCCCGGGAACCCCTATTTCCCATTTCACTCAGCTTCTTCTCCCCA |
| | AATGTTGTATTCATGTTCCTTTCCTGAAAGGGTGAGACATGGGAAAGAATTGTACTCCGT |
| | TCTAAGAAGTAAGTCCAAACCACCTGCCTATCTAAGATCTAGGAGATGGGGTCTGTGCCC |
| | CAGGCATGGGTGGCTGCAGCCCCTCACTCCCATTCTCACCAGAGACCTGGGGAGGCTGGC |
| | ATTTAGTGGAGGGGGGCACTGGCACATGTATGCTATCCTGGCTAATTAAAATCCCATCAG |
| | GATGGGTGTGCTGGGCTTGGACACCAGCATTCAAGAGGCAGAGGcGGGCAGATCTCTATG |
| | AGTTTGAAGCCATCCAGAGATACAAAGTGAGAGTCTATCTTTAAAAACAAACAAACAAAC |
| | AAACAAACAAACAAACAATCAAGTCAGATCCAGAACCAGTGAAGAGCAGCAAGGGGCCAT |
| | GATAGGCAAGACAAAGAGGCAGTTATCAGAGCAAGCCTTCTTGTTTATGCATTCCAGCTT |
| | GTTAACTAGCCATGCAGAAGCCCAACACCTCTGCCTTGGGTCAGAGAGGGCCAGCTTCGG |
| | CTCCTCAAACTGGAGTGGGATGGAAGCTTCTCCCCTCGAAAGTCAAGCACAGCTGCCATT |
| | ACCTACTAGGGCTGCAGGTTAGGCTGCTGAGCTCTGTGCATTTCAGGTTCATCCTTAACT |
| | TAAAATCAGAATAAGCCCGGGTTCCTCGGAGCCCACAGGAGTAGGATGTGGCTTGGAAGC |
| | TTCCTCCCTGACTATACCTGTCCCCACTTTGCTGAAGATGGATCAGAGCTCTCCCACCCC |
| | TGGCCCTGCCACTCCCCTCTGACACAGACACAGACACAGACACAGACACAGACACAGACA |
| | CAGACACAGACACAGACACAGACACAGACACAGACACAGACACAGACAGACACAGACACA |
| | GACAGACACAGACACAGACACAGACACAGACACAGACACAGACACAGACACAGACACAGA |
| | CACAGACACAGACACAGACACACAGGCATAGACACAGACACAGACAGACACAGACACAGA |
| | CACAGACACAGACACAGACACAGACACAGACACAGACACAGACACAGACACAGACACAGA |
| | CACAGACGACACAGACACAGACAGACACAGACACAGACAGACACAGACACAGACAGACAC |
| | AGACACAGACACAGACACAGACGACACAGACACAGACAGACACAGACACAGACACAGACA |
| | CAGACGACACAGACACAGACAGACACAGACACAGACACAGACACAGACACAGACACAGAC |
| | ACAGACACAGACACAGACACAGATGACACAGACACAGACGACACAGACACAGTCACAGAC |
| | ACAGACACAGACGACACAGACACAGACAGACACAGACACAGACAGACACAGACACAGACG |
| | ACACAGACACAGACACAGACACAGACGACACAGACACAGACAGACACAGACACAGACACA |
| | GACAGACACAGACACAGACAGACACAGACACAGACACAGACACAGACACAGACACAGACG |
| | ACACAGACACAGACAGACACAGACACAGACACAGACACAGACATAGACACAGACACAGAG |
| | ACACAGACACAGACAACACAGACACAGACACAGACACAGACACAGACACAGACTCAGACA |
| | CAGACACAGACACAGACACAGACACAGACACAGACTCAGACTCAGACTCAGACTCAGACT |
| | CAGACTCAGACTCAGACTCAGACTCAGACACACAGTCACACAGACACACACAGACACACA |
| | CAGACACACACACAAAGGCACACACACACACAAAGGCACACACACACACACACACCCCAC |
| | CGCCTGCCCCAATCTGCACTGCTGTAGCTCTACTTCCAGGAACCTGCAAGATCCCAAATG |
| | GTGCTTCCTGCATGAGGTAGCAGACAGGTGAGAACTTGAAGCCTGAGTGCTGTCTGCTTG |
| | GTCTGAAGCCTGCTGGCCTGGAAGGTCTGTGTTTTGGGCCTAACTGCTCTGGGTGGAGCT |
| | CAGAAAACATCCCTGGGTCTTTCCTGCTATTGGGAAATTTGTTCACGATGGCTAACTTAG |
| | GTGGGTTTTAGGCCATGGAGGTGAGAGGGCCTTGAGACCATAGAGGGGTTAGGAGCCTAT |
| | ACAGCAGAGTAGATGCCAAGCGCCAGGCCCTCCTCTAGGCCTCCCACTCAATACCCTGCT |
| | TCACCCCCACCTCACACCTTCCTTCCTCATGAGAACCATTTCCAAGGCTTGCTTCTTTCG |
| | GGAAGACTATCCAGATTAACCTATCTGCTCTCCAAACTGGTATTATACCTGTAAGCAGTG |
| | TTGTCTCTCAGAATGATAATGATAGTGATCTTATGTTGATGAAAAGACTGACAGTGACAG |
| | TCATGATGACAAAAGGTCTCCTCAGCTCTGGGTATATTAAAAATCACACCTGTGCCTGTG |
| | CCTGTGCTTAGAAGCATTCTTTATGGGTATTTGGATGCAGAGCAGAGGTCAAGAGAAAAG |
| | GAGTTTTGGCTTTATCCAGGACCAATAAACCAGCAGGGCATGGGACCCGAGCATGAGCCA |
| | CCATTTTTAGGAATTTTAGGGTTTTTGGCCCAATTCTTACTAATTCACCTGCATATAAGG |
| | ATATGGGGTATAGGACCCTACATAGGAGAAACCAAGATCAGGGAAGAAATGCAGGTTCCG |
| | TGGTCTCCGACAGTGGAGATACTGGAAGTACTCACccactttacagcaatgatgagggtg |
| | gccgtgcctatgggaccggagtataccccgtaaccccagcggtcatgaaaagtccgcaca |
| | gcgatggtaaggacgccaagcattgtgaaggtcgatctctggggttcatcaaagtcggcc |
| | agtgCTGGGGAGAGGCATAGCTATGGTGAGCAGCGTCCCTCACATGGCTGTGCCTCCATC |
| | CTTGGGAACCTATTGGTATGTCCTCTCAATCTGTAGGGCCAGCCTGGTTTCCATAAGGTC |
| | TGAATTTTGGTTATTTGGAGGGAGTGGGTGATGCTGCTTCCCTGGAGCAGGGTGGCTGAA |
| | ATAAACTGGTAGACTGAGTGACCAGCATTTCCTAGGAATCCTGAGACAAAAGTGTTAAGA |
| | CTAATGATTGGTGCGCAGAGCTGAGTCTCAGGAGGGACCCCGGGACTGCATCCCTGGGAG |
| | ACAAGGGTGAGCTTGCTTGGTTTCTCCCTTTTCTCTTTCCTTCCCTTCCCTTTCTTTCCC |
| | CTTTCTTTGCTCTCTCCCTCCTTCTCTCCCTGTCTCCCTTCCTCCCTCCCTCCCTGTCTC |
| | CTTTCCTTTCTTCTTCCTTCATCCTTTCTGCTTTCTACTTTTCTCTATCTCTTCCTTCTT |
| | TTTCTTTCAAAATTTTGCAGTTGCTGGTAATGGAATGAAGGGCCTATTGATTACCAGGCG |
| | AGCGATCTGCCATTGAGCTGTATATACCCCAGGTCCAAGGTGAGGATTTTGAATGGTCTG |
| | CCTTCCTAATACACAGAGCTGAGCTGACCCATGAGGGCAAATGCTCCTCTGAGCCTGGAG |
| | GACAAGCTGGGAGGCTAGGTCCAGGATGCCTTTGGCCTCTCCTTTGTATGCTTCTGTTTT |
| | TTAAATGTCACAAGTGCTAACTACTGGAGTCACTTAAGGATGGTGGAAATGAGAGTGCAG |
| | GCATCAGAGAAATGTGCATGTCTCTTTAAGCAGATTAAGCTCTGCAAAGCAGCAAGGAGG |
| | GAGGATCTCAGAGAGGGGCTGGGTACTGGCTGGGGTTCAGGACTGGCTCCCACCCATTGG |
| | CCAAGATGGCCACTTACccatcagggagacccacatgctcagggctgttccatagatgct |
| | gaagtactccagaatgtcacggcgcatgaagcacagcacagacaaaccaggcccatcaca |
| | ggcatgggagaaCTGTAGGGAAATCACATGAGGTCAGCAGGCAGTGGGCAGCCCAGGAGT |
| | GGGTGAGAACTGGTCCCAAGGCTCAGGTTCACTAGCTGTGAGCCCCTAATGGTTTTGTAC |
| | CTCAGCCTCCTCCCTCACACTATCAGAGCCCTTGTGGAGATTAAACAGGTGAGTCCATCT |
| | AGCCTGGGAGTGCAAAAGTCTTTGTAAATATCCCTTTCAGACTCAGCACTGGCCCAAGGC |
| | TGGTGAGAAGCATGCTCAGAAGGGCATCCTTAAAGACCACTTACacctttgcccatgact |
| | gactgaaagtgtacacattcctatgccagtctttgcataggagccttttatcctggaccc |
| | ctgtctctccataaaagaggaagcccttagattccccccaagcaagtgctgatTCTGACA |
| | CACTGGTTTCTTTCCCCCATATGCCAGCAGGTGTGTCCCTGACTCGTAGTTGAATAGATT |
| | TGCTTCTAAGCAAAAGGTTCTATATGCAGGATTTCCAAGCAGACAACTTATTTCTTGCAG |
| | AAAACAACTTGCTCTCCCTTTGCTTCACATTTCATCATTTTAAGTAATATTTAATTACAT |
| | GACATAATTATTTTGACAAGTGCAACTGGCACAAACAAGCCCAGCAGCCAGCACAATGAG |
| | CTCTTGGTAAGCCCAACTTAGCAGGAGGGAGCAGGCAAGCTGGAAAACAGCCTTGTCTGG |
| | AGGCAGCAGGGGCACCACCGAGGGAGGCAGGCGGAGAGCTGGGGACCCTGGATGATGGAT |
| | GTATCAGTCAAGCACATAGGGCCTACTTAGAAGCTCAGAGACCTCCTGCTGGTCACAGTT |
| | GCACATGGACTCTGTCAATCAATAGAGAGCATCCAGGGGAAGGGAGGAGGTGGTCCAGCC |
| | TCTGTGTTGGGTCAGCCCCAGCCTTGAGCTTTGGGTTCTGCACCTTTTAAAAGGGAGATT |
| | GGTGAAGAGGAGGTTAACCAACTAGGTATGAGCTCAGGAAAAGACAAGCTTTGGGTTGGG |
| | CCAGACCAAGGTACGCAAGTGGAGAAGGAAAGGAACTCAGCTCTGGGAGGGGACTCTGCT |
| | CTTCTGGCTCCTTACAGAACCACATGACCCCACCCCACCCCACCTGAGCCCATCATCTGT |
| | AGCATCTTGCTTCCTTCTCTTGTATAGGCCCCCATGAATGAGTAAAACTTACTTACTGTG |
| | AGATCCTGGGAAACACTCATGCCTTCCCCCACGAGGAGAAGAGCTTCCTTAGGCTTGATC |
| | TCAACATAGAATACTTGGCTACATGTGAAGGCCAGAGGAGCAGGCTTTCTAACAAGGGAT |
| | CTAACTGTCCTCAGGCCCTGAGGATTAATTTTTTGGGGGGTGGGTGACCTGTGTGACAGT |
| | GAACTTCCCTGGGGAACCTCCTGCCCAAGGAGGCAGGGGCAAGGCTGTGATGTGTACCCT |
| | TTCTCCCCAGAGGCAGGGAGATCTGGTCCAGCTGGTGCCAGGCTAGGACACAGCTGGGTG |
| | TGACAGGAGCCCTAACCCTGCTGTCAGCTCAGAGCTGGCAGAGGGGCCCAGGTTCTCTCA |
| | GGTCTCTCAGGCCCACCTTGTCTAATGGCATGAGAACACCTGTTCTGTGGGGCTTACAAG |
| | GGGACCCTAACGATAACTGCGGAGCATGGCACCCCACACTGCAAAAATGAAATGCTGTTT |
| | AAAGTTTGCTTTCATTAATCAAACTTTCCCCCAACCTGAAACCAAGTTAATATGTGCGTT |
| | ATGGGCATTTAAACAATGTGCTTGCCCTGGGCAGAATTAGCTCACCTCTGGGAAAAACAA |
| | TTCAATCGATCTTATTATGCTTTGCATTTCTGGTGGAGGACTCTAGTGAGTCTTTGTGAC |
| | TCTTTCATGCCCGACTCAGAACAGTATATGTTTGTGTGAGATGTGGTGACCAGGTCTAAG |
| | ACCACGTGTGTTAGAAACAGCAAGGTATGGAGACCATGTTGAAAGCAAAATGTGGGTGTA |
| | GGCTGATAATATCTGATTGTGGATTTGTGTGCTACTGAGTCAAAGGGCCAGAGAGACAGC |
| | TGTCTGCTATAAAAGCCTAAGACTCAGATCCCATTCTTTTTGTCCCTGTTTGTTGTGCTG |
| | TTCAGCAAGTAGAAAGGATGATATTGTCTAAGATTCTTAGATTAGAACCTGATTTTAGAT |
| | TAGATGACTATCAGGTTAGAACAGGAGAGGGCAGAATTCTTTGGAATACATCAGATCCAC |
| | CCGCTGTGTAACTGACACCAAGAGTCATTCTTCTATTCAGCAGCAGCATACCATACAACT |
| | GGTAGTTGTCATGGAGAGTCCTACAGCAGCCACGTGGAAGGCAGAACTCTGTGAGGAACA |
| | GATTGTGGCTTTGAGGCCAGAGGACATTTGTCATAAGAGACAGCTGGCCCTGCCACTCTG |
| | GGTGGGGTGTGGCAGGGTGGGCCTCCAAGGCCAGTGCAGAGGCAGCTGTAGGCCAATTAG |
| | ACCCAGGCAGGCAGGGGTGACCTGATTGGGGCTGTGATTTGCTGGACTGTATCTAACACA |
| | GGCCTTGGGAACAAGACCCTGGCTTATGTCCTTGACCGTGGGGTCTCATCTTGGCTCTGA |
| | CCTTGGCCAGGTCTCAAGAGGAACAAATGACAGTGTGGGACAAAGTACTGTGGGGCAGAC |
| | CAGGATCTGAGTGTTCATGGTGACACTGGTGGCCCAGTTTCTCTGAGACTCAGTTTCCTC |
| | TTCTATCAAATTGAAATCACTATGTTAGGCTCGTGGGTGATAATGAGTCCAACCCCACCA |
| | TGGTTGCTTTCTTGTGACTTATCATTGGCCTAATGTCCTCCCCTACTGAAGTGAACTCAA |
| | GAGCCATAGAGTTTCCAGTTCCTTGGGTTACCTATGGGACCACCACAACCAGGAGGTAGA |
| | CAGGTGCCAAGCCCTCCCCCACTGTTCTCAGCCCACATGCATTGTGGCTTCTCCCACCAC |
| | TAGAAAGTCATGCCAGCTGACTCAGGATATGGAACACGCATGTGAGCACAGATGTGTGAG |
| | TTTGTGGGCTCACTCATTGAGAGCCAGCTGGATACCTTCACATACTCTATGCCCTTGCCT |
| | TACTGAGACCTGCTGCAGGAAGGGCAGGCCTAAGGAGAGGATGCTAGTCTCTAAAAGTTT |
| | GGCTCTGCTCTAAGGAGGAGACTAGCAGGCTGCTTGCCAACCCTGAGCATGTATCCTACC |
| | AGTGTGTGGGCCTCACACCAGACAAACTAGTGAGGCATAGTGTGATGAGAGAGAAACGAA |
| | GGTTACAGAGTGGTAAAAGAGACAGTGTGACTCCTGGTTAGAGGATAGCTGAGAGGGCCA |
| | TCATGAGAGGTACTCAGAAGGACTAAAGGGCAAAGTGAGAGGAGGCCTTTAAGACAGAGA |
| | GTAGATGGGTAGATGAATGGACAGGGAGAGAGATGGTTGGTTAGCAGATAATAGAGAAAT |
| | GATAGACAGATAGACAGACAGACAGATGATGGATAGACACATAGAACAAGACAAATGATA |
| | AATGAATAGATGATAGACAAAGGAGATAGAGAGACAGAAGCAAGTTGAATGGGCAGGAAG |
| | ATAAAGTCAGGAAGACACAGAGCTCTGGTCAAGAACCCAGGGGAGAGCAGACCAGGGAGA |
| | AGAGGAGAGTGAACTCCTCGGGGGAGTGTAACTCTAGAAATCAGAAAAAAACAAAAAAAA |
| | AAAACCCCAAAAAACAAACAAACAAACAAACAAAAAAATTGGATACAGGCAGGAAGAAGG |
| | AAGAGATAGAGACTGGGAGAAACTAGACACAGAACCAGTCAAAGAAGCAGAGGGAGAGAG |
| | ACCCATGGCGGGAATAAAGAGAAGCAGAAACCCAGACACAAGGCTTCAGCAAAGCTGGGC |
| | CAGTGCCAGACATGCCCCGAACGAACGACAGAGGAGTCACCCAGTACTGTTGCCTGGGAA |
| | CAGAGTGGAGAAGGAACTAAGAGGCAGCCAGCCAGCTAGACACATAACAGGAAGAGAAAG |
| | AAGGACTCAGGGAGAGGCTGGCTCCTCTCAGTGGGGGTAGTTCCAAATTCTGGAGCTGCA |
| | GTCACCCAGGCCCTCTACCTTTCCTGAACCTAGTAGATCCATTCCTAGGCCTGCTCACTC |
| | ACCTTGTTCCTCCTCAGCTGAGCAACTCATGGAACAACGTTGGTAGAAAGGAGAGAGAGT |
| | CTGAGGAGCACCAGGCTTGACCTTAACTGACACCGGGCTCTCATGGGCCTGGCCTCAGTC |
| | TCAGGTGTCAATCACCCCCCTCAAATGTCTGGCGCACATGGAGAAACTGAGGTCCACAGA |
| | GGAAGACAGATTCCAGGAACCTTCTCTTCCCAGTCACCACCCCCACTGCTCCCCCAGACC |
| | CAGACTCTTTCTCTTCCAAATCCTGTTTCTGCATCACCTGGCACAGGACAATGGTGGTAA |
| | CCCTCCCGTGAGGACTTCCTCCTAATTTCCTCCTTCCACACTTACcgccacaaagaacat |
| | ggtgaagaggtagaccatggcctccatgtagaaacgcctcttggtagcgatgctcactgt |
| | cgggaggaaggccaggctgctgagggtaggcaggagcagtttggctacaactgtccccat |
| | ggaccaggaggaaggcactgactggggagaaggtggtaaaggcccccctggtctccaggg |
| | caggaagaaaaagagcccacttctttgcttctccagcagccctgaccgcagctgtggcag |
| | cacccacaaggagggcttaagtgctc (SEQ ID NO: 15) |
| Mouse | gagcacttaagccctccttgtgggtgctgccacagctgcggtcagggctgctggagaagc |
| (− strand, | aaagaagtgggctctttttcttcctgccctggagaccaggggggcctttaccaccttctc |
| reverse | cccagtcagtgccttcctcctggtccatggggacagttgtagccaaactgctcctgccta |
| complement) | ccctcagcagcctggccttcctcccgacagtgagcatcgctaccaagaggcgtttctaca |
| - start codon | tggaggccatggtctacctcttcaccatgttctttgtggcgGTAAGTGTGGAAGGAGGAA |
| is bold & | ATTAGGAGGAAGTCCTCACGGGAGGGTTACCACCATTGTCCTGTGCCAGGTGATGCAGAA |
| underlined; | ACAGGATTTGGAAGAGAAAGAGTCTGGGTCTGGGGGAGCAGTGGGGGTGGTGACTGGGAA |
| stop codon is | GAGAAGGTTCCTGGAATCTGTCTTCCTCTGTGGACCTCAGTTTCTCCATGTGCGCCAGAC |
| bold and | ATTTGAGGGGGGTGATTGACACCTGAGACTGAGGCCAGGCCCATGAGAGCCCGGTGTCAG |
| italicized | TTAAGGTCAAGCCTGGTGCTCCTCAGACTCTCTCTCCTTTCTACCAACGTTGTTCCATGA |
| | GTTGCTCAGCTGAGGAGGAACAAGGTGAGTGAGCAGGCCTAGGAATGGATCTACTAGGTT |
| | CAGGAAAGGTAGAGGGCCTGGGTGACTGCAGCTCCAGAATTTGGAACTACCCCCACTGAG |
| | AGGAGCCAGCCTCTCCCTGAGTCCTTCTTTCTCTTCCTGTTATGTGTCTAGCTGGCTGGC |
| | TGCCTCTTAGTTCCTTCTCCACTCTGTTCCCAGGCAACAGTACTGGGTGACTCCTCTGTC |
| | GTTCGTTCGGGGCATGTCTGGCACTGGCCCAGCTTTGCTGAAGCCTTGTGTCTGGGTTTC |
| | TGCTTCTCTTTATTCCCGCCATGGGTCTCTCTCCCTCTGCTTCTTTGACTGGTTCTGTGT |
| | CTAGTTTCTCCCAGTCTCTATCTCTTCCTTCTTCCTGCCTGTATCCAATTTTTTTGTTTG |
| | TTTGTTTGTTTGTTTTTTGGGGTTTTTTTTTTTTGTTTTTTTCTGATTTCTAGAGTTACA |
| | CTCCCCCGAGGAGTTCACTCTCCTCTTCTCCCTGGTCTGCTCTCCCCTGGGTTCTTGACC |
| | AGAGCTCTGTGTCTTCCTGACTTTATCTTCCTGCCCATTCAACTTGCTTCTGTCTCTCTA |
| | TCTCCTTTGTCTATCATCTATTCATTTATCATTTGTCTTGTTCTATGTGTCTATCCATCA |
| | TCTGTCTGTCTGTCTATCTGTCTATCATTTCTCTATTATCTGCTAACCAACCATCTCTCT |
| | CCCTGTCCATTCATCTACCCATCTACTCTCTGTCTTAAAGGCCTCCTCTCACTTTGCCCT |
| | TTAGTCCTTCTGAGTACCTCTCATGATGGCCCTCTCAGCTATCCTCTAACCAGGAGTCAC |
| | ACTGTCTCTTTTACCACTCTGTAACCTTCGTTTCTCTCTCATCACACTATGCCTCACTAG |
| | TTTGTCTGGTGTGAGGCCCACACACTGGTAGGATACATGCTCAGGGTTGGCAAGCAGCCT |
| | GCTAGTCTCCTCCTTAGAGCAGAGCCAAACTTTTAGAGACTAGCATCCTCTCCTTAGGCC |
| | TGCCCTTCCTGCAGCAGGTCTCAGTAAGGCAAGGGCATAGAGTATGTGAAGGTATCCAGC |
| | TGGCTCTCAATGAGTGAGCCCACAAACTCACACATCTGTGCTCACATGCGTGTTCCATAT |
| | CCTGAGTCAGCTGGCATGACTTTCTAGTGGTGGGAGAAGCCACAATGCATGTGGGCTGAG |
| | AACAGTGGGGGAGGGCTTGGCACCTGTCTACCTCCTGGTTGTGGTGGTCCCATAGGTAAC |
| | CCAAGGAACTGGAAACTCTATGGCTCTTGAGTTCACTTCAGTAGGGGAGGACATTAGGCC |
| | AATGATAAGTCACAAGAAAGCAACCATGGTGGGGTTGGACTCATTATCACCCACGAGCCT |
| | AACATAGTGATTTCAATTTGATAGAAGAGGAAACTGAGTCTCAGAGAAACTGGGCCACCA |
| | GTGTCACCATGAACACTCAGATCCTGGTCTGCCCCACAGTACTTTGTCCCACACTGTCAT |
| | TTGTTCCTCTTGAGACCTGGCCAAGGTCAGAGCCAAGATGAGACCCCACGGTCAAGGACA |
| | TAAGCCAGGGTCTTGTTCCCAAGGCCTGTGTTAGATACAGTCCAGCAAATCACAGCCCCA |
| | ATCAGGTCACCCCTGCCTGCCTGGGTCTAATTGGCCTACAGCTGCCTCTGCACTGGCCTT |
| | GGAGGCCCACCCTGCCACACCCCACCCAGAGTGGCAGGGCCAGCTGTCTCTTATGACAAA |
| | TGTCCTCTGGCCTCAAAGCCACAATCTGTTCCTCACAGAGTTCTGCCTTCCACGTGGCTG |
| | CTGTAGGACTCTCCATGACAACTACCAGTTGTATGGTATGCTGCTGCTGAATAGAAGAAT |
| | GACTCTTGGTGTCAGTTACACAGCGGGTGGATCTGATGTATTCCAAAGAATTCTGCCCTC |
| | TCCTGTTCTAACCTGATAGTCATCTAATCTAAAATCAGGTTCTAATCTAAGAATCTTAGA |
| | CAATATCATCCTTTCTACTTGCTGAACAGCACAACAAACAGGGACAAAAAGAATGGGATC |
| | TGAGTCTTAGGCTTTTATAGCAGACAGCTGTCTCTCTGGCCCTTTGACTCAGTAGCACAC |
| | AAATCCACAATCAGATATTATCAGCCTACACCCACATTTTGCTTTCAACATGGTCTCCAT |
| | ACCTTGCTGTTTCTAACACACGTGGTCTTAGACCTGGTCACCACATCTCACACAAACATA |
| | TACTGTTCTGAGTCGGGCATGAAAGAGTCACAAAGACTCACTAGAGTCCTCCACCAGAAA |
| | TGCAAAGCATAATAAGATCGATTGAATTGTTTTTCCCAGAGGTGAGCTAATTCTGCCCAG |
| | GGCAAGCACATTGTTTAAATGCCCATAACGCACATATTAACTTGGTTTCAGGTTGGGGGA |
| | AAGTTTGATTAATGAAAGCAAACTTTAAACAGCATTTCATTTTTGCAGTGTGGGGTGCCA |
| | TGCTCCGCAGTTATCGTTAGGGTCCCCTTGTAAGCCCCACAGAACAGGTGTTCTCATGCC |
| | ATTAGACAAGGTGGGCCTGAGAGACCTGAGAGAACCTGGGCCCCTCTGCCAGCTCTGAGC |
| | TGACAGCAGGGTTAGGGCTCCTGTCACACCCAGCTGTGTCCTAGCCTGGCACCAGCTGGA |
| | CCAGATCTCCCTGCCTCTGGGGAGAAAGGGTACACATCACAGCCTTGCCCCTGCCTCCTT |
| | GGGCAGGAGGTTCCCCAGGGAAGTTCACTGTCACACAGGTCACCCACCCCCCAAAAAATT |
| | AATCCTCAGGGCCTGAGGACAGTTAGATCCCTTGTTAGAAAGCCTGCTCCTCTGGCCTTC |
| | ACATGTAGCCAAGTATTCTATGTTGAGATCAAGCCTAAGGAAGCTCTTCTCCTCGTGGGG |
| | GAAGGCATGAGTGTTTCCCAGGATCTCACAGTAAGTAAGTTTTACTCATTCATGGGGGCC |
| | TATACAAGAGAAGGAAGCAAGATGCTACAGATGATGGGCTCAGGTGGGGTGGGGTGGGGT |
| | CATGTGGTTCTGTAAGGAGCCAGAAGAGCAGAGTCCCCTCCCAGAGCTGAGTTCCTTTCC |
| | TTCTCCACTTGCGTACCTTGGTCTGGCCCAACCCAAAGCTTGTCTTTTCCTGAGCTCATA |
| | CCTAGTTGGTTAACCTCCTCTTCACCAATCTCCCTTTTAAAAGGTGCAGAACCCAAAGCT |
| | CAAGGCTGGGGCTGACCCAACACAGAGGCTGGACCACCTCCTCCCTTCCCCTGGATGCTC |
| | TCTATTGATTGACAGAGTCCATGTGCAACTGTGACCAGCAGGAGGTCTCTGAGCTTCTAA |
| | GTAGGCCCTATGTGCTTGACTGATACATCCATCATCCAGGGTCCCCAGCTCTCCGCCTGC |
| | CTCCCTCGGTGGTGCCCCTGCTGCCTCCAGACAAGGCTGTTTTCCAGCTTGCCTGCTCCC |
| | TCCTGCTAAGTTGGGCTTACCAAGAGCTCATTGTGCTGGCTGCTGGGCTTGTTTGTGCCA |
| | GTTGCACTTGTCAAAATAATTATGTCATGTAATTAAATATTACTTAAAATGATGAAATGT |
| | GAAGCAAAGGGAGAGCAAGTTGTTTTCTGCAAGAAATAAGTTGTCTGCTTGGAAATCCTG |
| | CATATAGAACCTTTTGCTTAGAAGCAAATCTATTCAACTACGAGTCAGGGACACACCTGC |
| | TGGCATATGGGGGAAAGAAACCAGTGTGTCAGAatcagcacttgcttggggggaatctaa |
| | gggcttcctcttttatggagagacaggggtccaggataaaaggctcctatgcaaagactg |
| | gcataggaatgtgtacactttcagtcagtcatgggcaaaggtGTAAGTGGTCTTTAAGGA |
| | TGCCCTTCTGAGCATGCTTCTCACCAGCCTTGGGCCAGTGCTGAGTCTGAAAGGGATATT |
| | TACAAAGACTTTTGCACTCCCAGGCTAGATGGACTCACCTGTTTAATCTCCACAAGGGCT |
| | CTGATAGTGTGAGGGAGGAGGCTGAGGTACAAAACCATTAGGGGCTCACAGCTAGTGAAC |
| | CTGAGCCTTGGGACCAGTTCTCACCCACTCCTGGGCTGCCCACTGCCTGCTGACCTCATG |
| | TGATTTCCCTACAGttctcccatgcctgtgatgggcctggtttgtctgtgctgtgcttca |
| | tgcgccgtgacattctggagtacttcagcatctatggaacagccctgagcatgtgggtct |
| | ccctgatggGTAAGTGGCCATCTTGGCCAATGGGTGGGAGCCAGTCCTGAACCCCAGCCA |
| | GTACCCAGCCCCTCTCTGAGATCCTCCCTCCTTGCTGCTTTGCAGAGCTTAATCTGCTTA |
| | AAGAGACATGCACATTTCTCTGATGCCTGCACTCTCATTTCCACCATCCTTAAGTGACTC |
| | CAGTAGTTAGCACTTGTGACATTTAAAAAACAGAAGCATACAAAGGAGAGGCCAAAGGCA |
| | TCCTGGACCTAGCCTCCCAGCTTGTCCTCCAGGCTCAGAGGAGCATTTGCCCTCATGGGT |
| | CAGCTCAGCTCTGTGTATTAGGAAGGCAGACCATTCAAAATCCTCACCTTGGACCTGGGG |
| | TATATACAGCTCAATGGCAGATCGCTCGCCTGGTAATCAATAGGCCCTTCATTCCATTAC |
| | CAGCAACTGCAAAATTTTGAAAGAAAAAGAAGGAAGAGATAGAGAAAAGTAGAAAGCAGA |
| | AAGGATGAAGGAAGAAGAAAGGAAAGGAGACAGGGAGGGAGGGAGGAAGGGAGACAGGGA |
| | GAGAAGGAGGGAGAGAGCAAAGAAAGGGGAAAGAAAGGGAAGGGAAGGAAAGAGAAAAGG |
| | GAGAAACCAAGCAAGCTCACCCTTGTCTCCCAGGGATGCAGTCCCGGGGTCCCTCCTGAG |
| | ACTCAGCTCTGCGCACCAATCATTAGTCTTAACACTTTTGTCTCAGGATTCCTAGGAAAT |
| | GCTGGTCACTCAGTCTACCAGTTTATTTCAGCCACCCTGCTCCAGGGAAGCAGCATCACC |
| | CACTCCCTCCAAATAACCAAAATTCAGACCTTATGGAAACCAGGCTGGCCCTACAGATTG |
| | AGAGGACATACCAATAGGTTCCCAAGGATGGAGGCACAGCCATGTGAGGGACGCTGCTCA |
| | CCATAGCTATGCCTCTCCCCAGcactggccgactttgatgaaccccagagatcgaccttc |
| | acaatgcttggcgtccttaccatcgctgtgcggacttttcatgaccgctggggttacggg |
| | gtatactccggtcccataggcacggccaccctcatcattgctgtaaagtggGTGAGTACT |
| | TCCAGTATCTCCACTGTCGGAGACCACGGAACCTGCATTTCTTCCCTGATCTTGGTTTCT |
| | CCTATGTAGGGTCCTATACCCCATATCCTTATATGCAGGTGAATTAGTAAGAATTGGGCC |
| | AAAAACCCTAAAATTCCTAAAAATGGTGGCTCATGCTCGGGTCCCATGCCCTGCTGGTTT |
| | ATTGGTCCTGGATAAAGCCAAAACTCCTTTTCTCTTGACCTCTGCTCTGCATCCAAATAC |
| | CCATAAAGAATGCTTCTAAGCACAGGCACAGGCACAGGTGTGATTTTTAATATACCCAGA |
| | GCTGAGGAGACCTTTTGTCATCATGACTGTCACTGTCAGTCTTTTCATCAACATAAGATC |
| | ACTATCATTATCATTCTGAGAGACAACACTGCTTACAGGTATAATACCAGTTTGGAGAGC |
| | AGATAGGTTAATCTGGATAGTCTTCCCGAAAGAAGCAAGCCTTGGAAATGGTTCTCATGA |
| | GGAAGGAAGGTGTGAGGTGGGGGTGAAGCAGGGTATTGAGTGGGAGGCCTAGAGGAGGGC |
| | CTGGCGCTTGGCATCTACTCTGCTGTATAGGCTCCTAACCCCTCTATGGTCTCAAGGCCC |
| | TCTCACCTCCATGGCCTAAAACCCACCTAAGTTAGCCATCGTGAACAAATTTCCCAATAG |
| | CAGGAAAGACCCAGGGATGTTTTCTGAGCTCCACCCAGAGCAGTTAGGCCCAAAACACAG |
| | ACCTTCCAGGCCAGCAGGCTTCAGACCAAGCAGACAGCACTCAGGCTTCAAGTTCTCACC |
| | TGTCTGCTACCTCATGCAGGAAGCACCATTTGGGATCTTGCAGGTTCCTGGAAGTAGAGC |
| | TACAGCAGTGCAGATTGGGGCAGGCGGTGGGGTGTGTGTGTGTGTGTGTGCCTTTGTGTG |
| | TGTGTGTGCCTTTGTGTGTGTGTCTGTGTGTGTCTGTGTGTGTCTGTGTGACTGTGTGTC |
| | TGAGTCTGAGTCTGAGTCTGAGTCTGAGTCTGAGTCTGAGTCTGAGTCTGAGTCTGTGTC |
| | TGTGTCTGTGTCTGTGTCTGTGTCTGTGTCTGAGTCTGTGTCTGTGTCTGTGTCTGTGTC |
| | TGTGTCTGTGTTGTCTGTGTCTGTGTCTCTGTGTCTGTGTCTATGTCTGTGTCTGTGTCT |
| | GTGTCTGTGTCTGTCTGTGTCTGTGTCGTCTGTGTCTGTGTCTGTGTCTGTGTCTGTGTC |
| | TGTGTCTGTCTGTGTCTGTGTCTGTCTGTGTCTGTGTCTGTGTCTGTCTGTGTCTGTGTC |
| | GTCTGTGTCTGTGTCTGTGTCTGTGTCGTCTGTGTCTGTGTCTGTCTGTGTCTGTGTCTG |
| | TCTGTGTCTGTGTCGTCTGTGTCTGTGTCTGTGACTGTGTCTGTGTCGTCTGTGTCTGTG |
| | TCATCTGTGTCTGTGTCTGTGTCTGTGTCTGTGTCTGTGTCTGTGTCTGTGTCTGTGTCT |
| | GTGTCTGTCTGTGTCTGTGTCGTCTGTGTCTGTGTCTGTGTCTGTGTCTGTCTGTGTCTG |
| | TGTCGTCTGTGTCTGTGTCTGTGTCTGTGTCTGTCTGTGTCTGTGTCTGTCTGTGTCTGT |
| | GTCTGTCTGTGTCTGTGTCGTCTGTGTCTGTGTCTGTGTCTGTGTCTGTGTCTGTGTCTG |
| | TGTCTGTGTCTGTGTCTGTGTCTGTGTCTGTGTCTGTGTCTGTCTGTGTCTGTGTCTATG |
| | CCTGTGTGTCTGTGTCTGTGTCTGTGTCTGTGTCTGTGTCTGTGTCTGTGTCTGTGTCTG |
| | TGTCTGTGTCTGTGTCTGTGTCTGTCTGTGTCTGTGTCTGTCTGTGTCTGTGTCTGTGTC |
| | TGTGTCTGTGTCTGTGTCTGTGTCTGTGTCTGTGTCTGTGTCTGTGTCTGTGTCTGTGTC |
| | TGTGTCAGAGGGGAGTGGCAGGGCCAGGGGTGGGAGAGCTCTGATCCATCTTCAGCAAAG |
| | TGGGGACAGGTATAGTCAGGGAGGAAGCTTCCAAGCCACATCCTACTCCTGTGGGCTCCG |
| | AGGAACCCGGGCTTATTCTGATTTTAAGTTAAGGATGAACCTGAAATGCACAGAGCTCAG |
| | CAGCCTAACCTGCAGCCCTAGTAGGTAATGGCAGCTGTGCTTGACTTTCGAGGGGAGAAG |
| | CTTCCATCCCACTCCAGTTTGAGGAGCCGAAGCTGGCCCTCTCTGACCCAAGGCAGAGGT |
| | GTTGGGCTTCTGCATGGCTAGTTAACAAGCTGGAATGCATAAACAAGAAGGCTTGCTCTG |
| | ATAACTGCCTCTTTGTCTTGCCTATCATGGCCCCTTGCTGCTCTTCACTGGTTCTGGATC |
| | TGACTTGATTGTTTGTTTGTTTGTTTGTTTGTTTGTTTGTTTTTAAAGATAGACTCTCAC |
| | TTTGTATCTCTGGATGGCTTCAAACTCATAGAGATCTGCCCGCCTCTGCCTCTTGAATGC |
| | TGGTGTCCAAGCCCAGCACACCCATCCTGATGGGATTTTAATTAGCCAGGATAGCATACA |
| | TGTGCCAGTGCCCCCCTCCACTAAATGCCAGCCTCCCCAGGTCTCTGGTGAGAATGGGAG |
| | TGAGGGGCTGCAGCCACCCATGCCTGGGGCACAGACCCCATCTCCTAGATCTTAGATAGG |
| | CAGGTGGTTTGGACTTACTTCTTAGAACGGAGTACAATTCTTTCCCATGTCTCACCCTTT |
| | CAGGAAAGGAACATGAATACAACATTTGGGGAGAAGAAGCTGAGTGAAATGGGAAATAGG |
| | GGTTCCCGGGGCAAGACAGGGCCCATGGGTGATCTTCAATATTCTTCTCCTCTCTGGGGC |
| | CCCAGTGTTAGAACTGTGTGATTGATGGTCATACGGCCTACACTCAGGCTAATAGGCGGT |
| | GCCCATGTCACTGGCACTGCTCACTGGGACCTCCACTCCTGTTACTAATGGCAAGTTTTA |
| | GAACACTTGGAAACAGCAACATATCATCACTCAGAGTGACAAAGCTTGCCCTGATTTGCA |
| | CGAGGCCATTGACAGCATTTGTACATATAATGTATTTAGTGAGGTGTGCAATTAGGGCCG |
| | CTGTCCTGTGCCCACTGTGCACATCACTCAGTCAGGGCTGCTGTCCTGTGCCCTCTGTGC |
| | ACACCTGACAGTGGTATTTATAATGATCTTCCCCACTGAAACCTGAACAATCTTGAATAC |
| | CTAAAATACCTTTGGGCCCCAAGGATGTGCATTCCAGTGAGGAACCATGGGACTCCCATC |
| | TATTCCATGTAACACTCCATCTTACCAAGCTTGAACACACAGGGCCTATGAAACCAAGGT |
| | TAACAGAGGAAAACCCCTTAGCTGGGAAAACTGATCACGTCTGCCACTGAAGTGCCAAAG |
| | CCCACCAGATGCGGGCAGTAGAGGACCCAGGGGAAATTGGCCCAGAGAGTCCCATCAGTC |
| | AGGCATACAGAGGAATGGGGGTAGGGCTCTTCTACAGTTCATGGAGGGGTCCCTTCCTGT |
| | GCTGCACCCCAAGAACTTTGATGTCCCCACCTTGTGCCTGCAGctgaagaagatgaaaga |
| | gaagaagggcctgtaccccgacaagagcatctacacccagcagataggccccggcctgtg |
| | ctttggggccctggccctgatgcttcgattcttctttgagGTACCTGGCCTATGGGGAAG |
| | GGGAGCAAGAGCCTCTGGAAATAGTTAACTAACAGCCACATATGGAGGCTCCAAGTTGTA |
| | AATTCTGTCCTCTTGAGGATGGTACTCACCATCCCCAGTGTACTAGCCTCACTGCCTGAG |
| | TGGACAAGTCAGGCCCTGACTTTAGGAAGCCTGCACCCAAGATGCACGCATGTCCTGGAA |
| | GGAACCAGGAGCTGGTGCCCAGCACCCAGGAGGCATGGAACAGAGGAAGGCAGCAAGCCT |
| | TTCTGCCACCTTCTCAGGGTGGGCTGGCTGACAGAGGCACCAAATGGCTAGGACTTGGTT |
| | TAGGGAAGGGATGGAGGCCCAGACTGACTCATTCTCCTCCACAGgaatgggattacacct |
| | acgtccacagcttctaccactgtgccctggccatgtcctttgtcctgctgctgcccaagg |
| | tcaacaagaaggctgggaacgcaggggcccccgccaagctgaccttctccaccctctgct |
| | gcacttgtgtc [symbol] ctatacccccccacacacacacacacaccaggcccctgccttcctg |
| | cctggcagtcctgctgtctctcccaaggtacttcctatactttgttatgcggcctgtaca |
| | tgagaaatggtcttctctacaccccaagagaccagcaggcctgctgcattctgctgagtg |
| | ctgcttagggacccactggttctgtgttcaccagttgcttcactctgttcaggaaaaaaa |
| | agaactttatcccccaaggcctcacaaccataggtgtgcctggcagagaaccctagacca |
| | gtaaatacccagcagcatgcagggttatctatttcccaggtcctgcctgtcagaatcgtc |
| | tgctttccctaggaaactgggattgcccttttaacccctgcccaggctctgaaagctctc |
| | cacttaggaagctggagccagcgaaccttgcatacccctgcctgagtcccatcccttctg |
| | caggttttcgatcaagccattcacagtaaactcttgatcagccactagtcattagcttcg |
| | cctaacatacattctagttcctggaa (SEQ ID NO: 16) |

One or more modifications, in some instances, can include an insertion, a deletion, a substitution, or combinations thereof. In some embodiments, the polypeptide does not encompass one or more naturally occurring polypeptides (e.g., does not encompass one or more of the wt-myomaker polypeptides). In other embodiments, the polypeptide does not encompass any of the wt-myomaker polypeptides. In some embodiments, the polypeptide does not encompass any naturally occurring polypeptide (e.g., does not encompass any of the wt-myomaker polypeptides or any other naturally occurring polypeptide).

In some embodiments, one or more modifications to a wt-myomaker polypeptide can include one or more substitutions, one or more insertions, or one or more deletions (or combinations thereof) to one or more amino acids in a hydrophobic region of a wt-myomaker polypeptide, to one or more amino acids in a hydrophilic region of a wt-myomaker polypeptide, or in a combination thereof. In some embodiments, one or more modifications to a wt-myomaker polypeptide can include one or more substitutions or one or more deletions (or combinations thereof) to one or more amino acids in a hydrophobic region of a wt-myomaker polypeptide, to one or more amino acids in a hydrophilic region of a wt-myomaker polypeptide, or in a combination thereof.

In some embodiments, the myomaker polypeptide can have a polypeptide sequence with an amino acid sequence identity to a wt-myomaker polypeptide (e.g., SEQ ID NO:1, SEQ ID NO:2, SEQ ID NO:3, SEQ ID NO:4, SEQ ID NO:5, or SEQ ID NO:6) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. In some embodiments, the myomaker polypeptide sequence has an amino acid sequence identity to SEQ ID NO:1, SEQ ID NO:2, SEQ ID NO:3, SEQ ID NO:4, SEQ ID NO:5, or SEQ ID NO:6 of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. The amino acid sequence identity (e.g., percent identity) can be determined by any suitable method, such as using BLAST, BLAST-2, ALIGN, ALIGN-2, Clustal Omega, or Megalign software. Unless otherwise indicated, the amino acid sequence identity (e.g., percent identity) is determined using BLAST-2.

Nucleic acid molecules that encode for the myomaker polypeptide are termed “myomaker nucleic acid molecules.” In certain embodiments, the myomaker nucleic acid molecule is included in a vector (e.g., a viral vector, a retroviral vector, a lentiviral vector, a VSV vector, an adenoviral vector, an adeno-associated viral vector, a herpesviral vector, a chimeric viral vector, a plasmid, an expression vector, a conjugative vector, or a nonconjugative vector). In certain embodiments, the myomaker nucleic acid molecule is in a cell, such as an insect cell (e.g., an Sf9 cell) or mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, or an adipose stem cell).

In other embodiments, the myomaker nucleic acid molecule comprises one or more nucleic acid sequences that are not used to encode for the myomaker polypeptide (e.g., one or more introns). For example, the myomaker nucleic acid molecule can include one or more nucleic acid molecules as found in nature (e.g., including introns). In certain embodiments, the myomaker nucleic acid molecule differs from the one or more nucleic acid molecules in nature because the myomaker nucleic acid molecule does not include one or more introns. In some embodiments, the myomaker nucleic acid molecule is a cDNA molecule (“myomaker cDNA molecule”). In certain embodiments, the myomaker cDNA molecule is identical to a nucleic acid molecule found in nature. In other embodiments, the myomaker cDNA molecule is not identical to a nucleic acid molecule found in nature (e.g., due to the myomaker cDNA molecule not including one or more introns in the nucleic acid molecule found in nature).

In some embodiments, the myomaker nucleic acid molecule sequence has a sequence identity to a nucleic acid molecule encoding a wt-myomaker polypeptide (e.g., SEQ ID NO:7, SEQ ID NO:10, SEQ ID NO:13, SEQ ID NO:14, SEQ ID NO:15, or SEQ ID NO:16) of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. In some embodiments, the myomaker nucleic acid molecule sequence has a sequence identity to SEQ ID NO:7, SEQ ID NO:10, SEQ ID NO:13, SEQ ID NO:14, SEQ ID NO:15, or SEQ ID NO:16 of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. Nonlimiting examples of wt-myomaker polypeptides and wt-myomaker nucleic acid molecules can be found in Table 2. The nucleic acid sequence identity (e.g., percent identity) can be determined by any suitable method, such as using BLAST, BLAST-2, ALIGN, ALIGN-2, Clustal Omega, CRISPor Megalign software. Unless otherwise indicated, the nucleic acid sequence identity (e.g., percent identity) is determined using BLAST-2.

In some embodiments, the myomaker nucleic acid molecule encodes for a myomaker polypeptide that has one or more modifications to wt-myomaker polypeptide in a hydrophobic region, in a hydrophilic region, or in a combination thereof.

The myomaker nucleic acid molecule can be made using any suitable technique, such as but not limited to, those found in WO 2014/210448 A1, those found in WO 2018/152103 A1, chemical synthesis, enzymatic production or biological production. Chemical synthesis of a nucleic acid molecule can include, for example, a nucleic acid molecule made by in vitro chemical synthesis using phosphotriester, phosphite or phosphoramidite chemistry and solid phase techniques, or via deoxynucleoside H-phosphonate intermediates. Enzymatically produced nucleic acid molecules can be accomplished using any suitable method including but not limited to Polymerase Chain Reaction (PCR). Biologically produced nucleic acid molecules can be accomplished using any suitable method including but not limited to a recombinant nucleic acid produced (i.e., replicated) in a living cell, such as a recombinant DNA vector replicated in bacteria.

Modifications or changes made in the structure of the myomaker nucleic acid molecules and/or myomaker polypeptides can be used in the present invention. In certain embodiments, a myomaker polypeptide can be modified (e.g., by one or more insertions, one or more deletions, or one or more substitutions (e.g., conservative substitutions)). In some embodiments, the myomaker polypeptide which was modified does not have an appreciable loss (e.g., a decrease in a function of less than about 1%, less than about 5%, less than about 10%, less than about 25%, less than about 50%, less than about 75%, less than about 90%, less than about 95%, less than about 99%, or less than about 100%) of one or more functions of the unmodified myomaker polypeptide such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the myomaker polypeptide which was modified retains desired levels (e.g., at least about 20%, at least about 40%, at least about 50%, at least about 75%, at least about 80%, at least about 90%, at least about 95%, or at least about 99%) of one or more functions of the unmodified myomaker polypeptide, such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the myomaker polypeptide after modification has an increased level of one or more functions as compared to the unmodified myomaker polypeptide. Nucleic acid molecules can be designed to encode for such a modified myomaker polypeptide, and such nucleic acid molecules can be used in the present invention.

A “functional myomaker polypeptide” is defined as a myomaker polypeptide (e.g., a modified polypeptide) that has desired levels (e.g., at least about 20%, at least about 40%, at least about 50%, at least about 75%, at least about 80%, at least about 90%, at least about 95%, or at least about 99%, as compared to another myomaker polypeptide, such as a naturally occurring myomaker polypeptide) of one or more functions such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the function myomaker polypeptide has an increased level of one or more functions as compared to another myomaker polypeptide (e.g., a naturally occurring myomaker polypeptide). Nucleic acid molecules can be designed to encode for functional myomaker polypeptides, and such nucleic acid molecules can be used in the present invention.

A “functionally equivalent myomaker polypeptide” is defined as a myomaker polypeptide that has been modified (e.g., by one or more insertions, one or more deletions, or one or more substitutions (e.g., conservative substitutions)) from an original myomaker polypeptide and that modified myomaker polypeptide retains desired levels (e.g., at least about 20%, at least about 40%, at least about 50%, at least about 75%, at least about 80%, at least about 90%, at least about 95%, or at least about 99%) of one or more functions of the original myomaker polypeptide, such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the functionally equivalent myomaker polypeptide can have an increased level of one or more functions compared to the original myomaker polypeptide. Nucleic acid molecules can be designed to encode for functionally equivalent myomaker polypeptides, and such nucleic acid molecules can be used in the present invention.

In certain embodiments, the shorter the length of a myomaker polypeptide, the fewer the modifications (e.g., substitutions) that can be made within the polypeptide while retaining, for example, a desired level of a chosen function. In some instances, longer domains can have a greater number of such changes while retaining, for example, a desired level of a chosen function. In other embodiments, a full-length polypeptide can have more tolerance for a fixed number of changes while retaining, for example, a desired level of a chosen function, as compared to a shorter length of that polypeptide.

The design of substitutions can take many forms, including but not limited to those described herein. In some embodiments, the hydropathic index of amino acids may be considered in designing substitutions. In the hydropathic index, each amino acid is assigned a hydropathic index on the basis of their hydrophobicity or charge characteristics, as follows: isoleucine (+4.5); valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); or arginine (−4.5). In some instances, certain amino acids may be substituted for other amino acids having a similar hydropathic index. In making changes based upon the hydropathic index, the substitution of amino acids with hydropathic indices can be made with amino acids that have an index difference of no more than ±2, no more than ±1, or no more than ±0.5.

In some embodiments, substitutions can also be made based on hydrophilicity values. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids with hydrophilicity values can be made with amino acids that have a value of no more than ±2, no more than ±1, or no more than ±0.5.

A “conservative substitution” in an amino acid sequence or polypeptide indicates that a given amino acid residue is replaced by a residue having similar physiochemical characteristics (e.g., no more than ±1 when based on hydropathic index or no more than ±1 when base on hydrophilicity values). Examples of conservative substitutions include (a) substitution of one aliphatic residue for another with an aliphatic residue, (b) substitution of one of Ile, Val, Leu, or Ala for one another of Ile, Val, Leu, or Ala, (c) substitution of one of Gly, Ile, Val, Leu, or Ala for one another of Gly, Ile, Val, Leu, or Ala, (d) substitution of one polar residue for another polar residue, (e) substitution of one of Lys and Arg with another of Lys and Arg, (f) substitution of one of Glu and Asp with another of Glu and Asp, (g) substitution of one of Gln and Asn with another of Gln and Asn, (h) substitution of one hydroxyl or sulfur containing residue with another hydroxyl or sulfur containing residue, (i) substitution of one of Ser, Cys, Thr, or Met with another of Ser, Cys, Thr, or Met, (j) substitution of one aromatic residue for another with an aromatic residue, (k) substitution of one of Phe, Tyr, or Trp with another of Phe, Tyr, or Trp, (1) substitution of one basic residue for another basic residue, (m) substitution of one of His, Lys, or Arg with another of His, Lys, or Arg, (n) substitution of an acidic/amide residue with another acidic/amide residue, (o) substitution of one of Asp, Glu, Asn, or Gln with another of Asp, Glu, Asn, or Gln, (p) substitution of a residue with another residue of a similar size, and (q) substitution of one of Ala, Gly, or Ser with another of Ala, Gly, or Ser. In some embodiments, each amino acid in a hydrophobic region of a polypeptide can be substituted with conservative substitutions (e.g., any combination of conservative substitutions relating to hydrophobic residues).

While discussion has focused on amino acid changes, it will be appreciated that these changes may occur by alteration of the encoding DNA; taking into consideration also that the genetic code is degenerate and that two or more codons may code for the same amino acid. Tables A1 and B1 of amino acids and their codons are presented herein for use in such embodiments, as well as for other uses, such as in the design of probes and primers and the like.

Tables A1 and B1. Amino Acid Designations and Codon Table

| Table A1- Amino Acid | | | Table B1 - Codons for |
|---|---|---|---|
| Designations | | | Amino Acids |
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The term “functionally equivalent codon” is used herein to refer to codons that encode the same amino acid, such as the six codons for arginine or serine.

In certain instances, the nucleic acid molecule can be engineered to contain distinct sequences while at the same time retaining the capacity to encode a desired polypeptide. In some embodiments, this can be accomplished owing to the degeneracy of the genetic code (i.e., the presence of multiple codons) which encode for the same amino acids. In other instances, it can be accomplished by including, adding, or excluding introns in the nucleic acid molecule.

In certain embodiments, a restriction enzyme recognition sequence can be introduced into a nucleic acid sequence while maintaining the ability of that nucleic acid molecule to encode a desired polypeptide. In other embodiments, a CRISPR system (e.g., a CRISPR system comprising one or more of guide RNA, crRNA, tracrRNA, sgRNA, DNA repair template, and Cas protein, such as but not limited to CRISPR/Cas9) can be used to introduce a nucleic acid molecule while maintaining the ability of that nucleic acid molecule to encode a desired polypeptide.

It will also be understood that amino acid sequences (e.g., polypeptides) and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological activity where polypeptide expression is concerned. The addition of terminal sequences particularly applies to nucleic acid sequences that may, for example, include various non-coding sequences flanking either of the 5′ or 3′ portions of the coding region or may include various internal sequences, (i.e., introns) which can occur within genes.

Some embodiments use synthesis of polypeptides in cyto, via transcription and translation of appropriate nucleic acid molecules (e.g., nucleic acid sequences as discussed herein). These polypeptides will include the twenty “natural” amino acids, and post-translational modifications thereof. In vitro peptide synthesis permits the use of modified or unusual amino acids. In some embodiments, the myomaker polypeptide encompasses modifications (e.g., one or more substitutions or one or more insertions) that include one or more modified or unusual amino acids. A table of exemplary, but not limiting, modified or unusual amino acids is provided in Table C1.

TABLE C1
Modified or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, | AHyl | allo-Hydroxylysine |
| | beta-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

The presently disclosed subject matter further includes a method of producing a myomaker polypeptide (e.g., a mutant myomaker polypeptide or a wt-myomaker polypeptide). Any suitable method can used to make the myomaker polypeptides including but not limited to expression through any suitable molecular biological technique (e.g., using a prokaryotic or eukaryotic expression system), isolation from a source in nature, or chemical synthesis. Eukaryotic expression systems include plant-based systems; insect cell systems via recombinant baculoviruses; whole insect systems via recombinant baculoviruses; genetically engineered yeast systems, including but not limited to Saccharomyces sp. and Pichia spp.; and mammalian cell systems, including but not limited to C2C12 cells, 10T ½ fibroblasts, NIH/3T3 fibroblasts, mesenchymal stem cells (MSCs), hematopoietic stem cells, Chinese hamster ovary cells or other cell lines commonly used for industrial scale expression of recombinant proteins. In some embodiments, useful plant-based expression systems can include transgenic plant systems. In some embodiments, useful plant-based expression systems can include transplastomic plant systems.

In some embodiments, a method of producing the myomaker polypeptide includes providing a host cell comprising a myomaker nucleic acid molecule, as disclosed herein, operatively linked to a promoter operable under conditions whereby the encoded myomaker polypeptide is expressed; and recovering the myomaker polypeptide from the host cell.

Myomerger Polypeptides, Nucleic Acid Molecules, and Compositions

Some embodiments of the invention include pseudotyped particles comprising the myomerger polypeptide, the myomerger nucleic acid molecule, or both, compositions comprising the pseudotyped particles, or uses of the pseudotyped particles. In some embodiments, the myomerger polypeptide is the myomerger protein disclosed in WO 2019/241622 A1, which is herein incorporated by reference in its entirety. In other embodiments, myomerger polypeptide is the myomerger protein disclosed in Table 1A of WO 2019/241622 A1.

In some embodiments, the extracellular myomerger polypeptide is the extracellular portion of a myomerger polypeptide (e.g., when it is membrane-bound, the extracellular portion is outside of the cell/liposome, and not in the membrane; including but not limited to disclosed in Table 1D of WO 2019/241622 A1). In other embodiments, the extracellular myomerger polypeptide is the ectodomain of a myomerger polypeptide. In some embodiments, the myomerger polypeptide or the extracellular myomerger polypeptide can be a polypeptide that (a) induces fusogenicity (e.g., by inducing the fusion of myomaker-expressing fibroblasts), (b) can confer fusogenic activity to normally non-fusogenic cells, (c) is expressed during developmental myogenesis, (d) is expressed during regenerative myogenesis, (e) is expressed only during developmental myogenesis, (f) is expressed only during regenerative myogenesis, (g) permeabilizes the membrane of a cell or liposome (h) lyses a cell or liposome, (i) forms pores in a cell membrane or liposome membrane, (j) stresses a membrane (e.g., cell membranes or liposome membranes) or (k) combinations thereof.

The term “myomerger polypeptide” encompasses “wt-myomerger polypeptides” (i.e., myomerger polypeptides found in nature without any purposely human-made modification) and “mutant myomerger polypeptides” (e.g., with one or more modifications made to a wt-myomerger polypeptide). The term “extracellular myomerger polypeptide” encompasses “extracellular wt-myomerger polypeptides” (i.e., extracellular portions of myomerger polypeptides made from wt-myomerger polypeptides) and “mutant extracellular myomerger polypeptides” (e.g., with one or more modifications made to an extracellular wt-myomerger polypeptide).

Nonlimiting examples of wt-myomerger polypeptides are found in Table 2A. In other embodiments, the myomerger polypeptide has at least one amino acid modification relative to a wt-myomerger polypeptide. A wt-myomerger polypeptide can, in some embodiments, be a myomerger polypeptide from any animal including but not limited to a mammal, a rat, a cat, a rabbit, a human, a cow, a chicken, a turkey, a monkey, a tree shrew, a dog, a pig, a shrew, an elephant, or an opossum. Table 2A provides nonlimiting examples of wt-myomerger polypeptides and Tables 2B and 2C provide nonlimiting examples of related nucleic acid sequences (including start and stop codons).

TABLE 2A
Myomerger

| Source | Polypeptide sequence |
|---|---|
| Mouse | MPEESCTVKLIQLKTGEYRGAGPAMPVPLLPMVLRSLLSRLLLPVARLA |
| (long) | RQHLLPLLRRLARRLSSQDMREALLSCLLFVLSQQQPPDSGEASRVDHS |
| | QRKERLGPQK (SEQ ID NO: 17) |
| Mouse | MPVPLLPMVLRSLLSRLLLPVARLARQHLLPLLRRLARRLSSQDMREAL |
| (short) | LSCLLFVLSQQQPPDSGEASRVDHSQRKERLGPQK (SEQ ID NO: 18) |
| Human | MPTPLLPLLLRLLLSCLLLPAARLARQYLLPLLRRLARRLGSQDMREAL |
| | LGCLLFILSQRHSPDAGEASRVDRLERRERLGPQK (SEQ ID NO: 19) |
| Cat | MPAPLLPLLLRTLMSRLLLPATRLARRHLLPLLRRLARRLGSQDVREAL |
| | LGCLLFILSQSRPPDAEEVSRVAGQERRERLAPPK (SEQ ID NO: 20) |
| Rabbit | MPAPLLPLLLRTLLSRLLLPAARLARRHLLPLLRRLAQRLGSQGTREALL |
| | GCLLFVLSQRQPPDASGEASRVDPPERKERLGRQK (SEQ ID NO: 21) |
| Dog | MPAPLLPLLLRTLVSRLLLPAARLARRHLLPLLRGLARRLGSQEVREAL |
| | LGCLLFILSQRHPPDAEEASRVAGQERKERLAPPK (SEQ ID NO: 22) |
| Elephant | MPVPLLSLLLRALLSRLLLPAARLARQHLLPLLRRLARRLGSQDMRQAL |
| | LGCLLFVLSQQHPPDAGEASREALSERRGRLAPOK (SEQ ID NO: 23) |

TABLE 2B
Myomerger

| Source | cDNA nucleic acid sequence |
|---|---|
| Mouse (long) | atgcc agaagaaagc tgcactgtaa aactaatcca gttgaaaact ggggagtaca gaggtgcagg |
| | tcctgccatg cccgttccat tgctcccgat ggtgcttcga tcgctgctgt cccgcctgct gctgcctgtt |
| | gcccgcctgg cccggcagca cctcctgccc ttgctgcgcc ggctggcccg ccgactgagc |
| | tcccaagaca tgagagaggc tctgctgagc tgtctgctct ttgtcctcag ccagcaacag |
| | ccaccggatt ctggagaggc ctccagagtg gaccactccc agaggaagga gagattgggc |
| | ccccagaagt ga (SEQ ID NO: 24) |
| Mouse (short) | atgcccg ttccattgct cccgatggtg cttcgatcgc tgctgtcccg cctgctgctg cctgttgccc |
| | gcctggcccg gcagcacctc ctgcccttgc tgcgccggct ggcccgccga ctgagctccc |
| | aagacatgag agaggctctg ctgagctgtc tgctctttgt cctcagccag caacagccac |
| | cggattctgg agaggcctcc agagtggacc actcccagag gaaggagaga ttgggccccc |
| | agaagtga (SEQ ID NO: 25) |
| Human | atgcccac gccactgctc ccgctgctgc ttcgattgct gctgtcctgc ctgctgctgc ctgctgcccg |
| | cctggcccgc caatacctcc tgcccctgct gcgccgattg gcccgccgcc tgggctccca |
| | ggacatgcga gaggctttgc tgggctgtct gctgttcatt ctcagccagc gacactcgcc |
| | agacgctggg gaggcctcaa gagtggaccg cctggagagg agggagaggt taggccccca |
| | aaagtga (SEQ ID NO: 26) |
| Cat | atgcccgc tccactgctc ccactgctgc ttcgaaccct gatgtcccgc ttgctgctgc ctgccacccg |
| | cctggcccgc cggcacctcc tgcccctcct gcgccgactg gcccgccgcc tgggctcgca |
| | ggatgttcga gaagctttgc tgggctgtct gttgttcatc ctcagccaga gccgcccgcc |
| | cgacgctgag gaggtctcca gagtggctgg ccaggagagg agggagaggc tagctccccc |
| | aaaatga (SEQ ID NO: 27) |
| Rabbit | atgcc tgcccccctg ctgccgctgc tgctgcgaac gctgctgtcc cgtctgctgc tgcccgctgc |
| | ccgcctggcc cgccggcacc tcctgcccct gctgcgccga ctggctcaac gcctgggctc |
| | ccagggcacg cgcgaggctt tgctgggctg tttgctgttt gtcctcagcc agagacagcc |
| | gccagatgcc tctggggagg cctccagagt ggacccaccg gagaggaagg agaggttagg |
| | ccgccaaaag tga (SEQ ID NO: 28) |
| Dog | atgc ctgctccact gctcccactg ctgctgcgaa cgctggtgtc tcgcctgctg ctgcctgctg |
| | cccgcctggc ccggcggcac ctcctgcccc tgctgcgtgg actggcccgc cgcctaggct |
| | cgcaggaggt tcgagaggct ttgctgggct gtctgttgtt catcctcagc cagagacatc |
| | cgccggacgc cgaggaggcc tccagagtgg ctggccagga gaggaaggag aggctagctc |
| | cccccaaatg a (SEQ ID NO: 29) |
| Elephant | atgcccgtcc cgctgctctc gctgctgctg cgcgcgctgc tgtcccgcct gctgctgcct |
| | gctgcccgcc tggcccgcca gcacctcctg cccctcctgc gccgacttgc tcgccgcctg |
| | ggctcccagg acatgcgaca ggctctcttg ggatgtctgc tctttgtcct cagccagcaa |
| | cacccgccgg acgctggtga ggcctccaga gaggccctct cagagaggag agggaggcta |
| | gccccccaaa agtga (SEQ ID NO: 30) |

TABLE 2C
(exons in lowercase) - Myomerger

| Source | Genomic nucleic acid sequence |
|---|---|
| Human | ctgcccggtgagagctgccgtggattggtggggGTAGGGGACTGAGAGGTCAGGGAGTGT |
| (+ strand) - | CAGGTCAGGGTGGATCAGGAGCCCCAAAAGAAAAATTGAGAATTGCCTGGAGAAGAACTC |
| start codon is | CTGCTAGACTGAGGGAGAAGGGTTAGGGAACTCCAGGGGCATTGAGGCTGTGCAAGAGGA |
| bold & | GGGGGTGACTAGAGGAAGGGAGGGGCCAGGGAGCAGTAGGAATGCCTGGAGCTGGGAACG |
| underlined; | GCAAGCTGTAGGTCTTGGTTTACTCTTGCCTTGGTTCAGTCTCCCCATCTGTGCTATGGT |
| stop codon is | GAGAACCTTCCTGCCTCAGCTGCCTTGCCAAGAGAAAGGGCTTCATGAAAGCAAAAATGA |
| bold and | CCTACAAATTGAGGTCAGGAGCAGGAAGGTGTAAACTGAAGGGAGGGGGAACTCCTGCCC |
| italicized | ACCCCATGTCCTTGCCAGGTGAGGCAGAACCAGGACATGCAAGCCTAAAGTCTGTGTTGT |
| | CTTCCCAGgcactgactcactggccctgccatgcccacgccactgctcccgctgctgctt |
| | cgattgctgctgtcctgcctgctgctgcctgctgcccgcctggcccgccaatacctcctg |
| | cccctgctgcgccgattggcccgccgcctgggctcccaggacatgcgagaggctttgctg |
| | ggctgtctgctgttcattctcagccagcgacactcgccagacgctggggaggcctcaaga |
| | gtggaccgcctggagaggagggagaggttaggcccccaaaag [symbol] ggccacaagtcctgg |
| | cagcagctgtatccacaaaatgctttcttttggagtaggataatcctggcaccagcactg |
| | accgaagcctgcccagtggacagaagatatagtgagggttgtgcatgagagggatctgcc |
| | acagacatgcctctccactcccaacagaaatgtctttctggaagaatgccttgcatctag |
| | cacaaaactgattattgcccctctgtcctccagcagttcctcccaaagaccactcctaat |
| | cacctctggcctcaggcgggaggggaactaacacccacccacccctgccctccctgcaaa |
| | tgggaacatcaaggttcccagtgcttaactgagggacaagtgacaatttagcagagaggc |
| | aagatttgaatccagactgtcttccagactcaggacctaccttaaaataatatctgagtt |
| | gcttatggaggcagacctgcctgcaaagcccagcactcagcaagtgctcaataaatattt |
| | gatttgaattctttc (SEQ ID NO: 31) |
| Human | gaaagaattcaaatcaaatatttattgagcacttgctgagtgctgggctttgcaggcagg |
| (− strand, | tctgcctccataagcaactcagatattattttaaggtaggtcctgagtctggaagacagt |
| reverse | ctggattcaaatcttgcctctctgctaaattgtcacttgtccctcagttaagcactggga |
| complement) | accttgatgttcccatttgcagggagggcaggggtgggtgggtgttagttcccctcccgc |
| | ctgaggccagaggtgattaggagtggtctttgggaggaactgctggaggacagaggggca |
| | ataatcagttttgtgctagatgcaaggcattcttccagaaagacatttctgttgggagtg |
| | gagaggcatgtctgtggcagatccctctcatgcacaaccctcactatatcttctgtccac |
| | tgggcaggcttcggtcagtgctggtgccaggattatcctactccaaaagaaagcattttg |
| | tggatacagctgctgccaggacttgtggcctcacttttgggggcctaacctctccctcct |
| | ctccaggcggtccactcttgaggcctccccagcgtctggcgagtgtcgctggctgagaat |
| | gaacagcagacagcccagcaaagcctctcgcatgtcctgggagcccaggcggcgggccaa |
| | tcggcgcagcaggggcaggaggtattggcgggccaggcgggcagcaggcagcagcaggca |
| | ggacagcagcaatcgaagcagcagcgggagcagtggcgtgggcatggcagggccagtgag |
| | tcagtgcCTGGGAAGACAACACAGACTTTAGGCTTGCATGTCCTGGTTCTGCCTCACCTG |
| | GCAAGGACATGGGGTGGGCAGGAGTTCCCCCTCCCTTCAGTTTACACCTTCCTGCTCCTG |
| | ACCTCAATTTGTAGGTCATTTTTGCTTTCATGAAGCCCTTTCTCTTGGCAAGGCAGCTGA |
| | GGCAGGAAGGTTCTCACCATAGCACAGATGGGGAGACTGAACCAAGGCAAGAGTAAACCA |
| | AGACCTACAGCTTGCCGTTCCCAGCTCCAGGCATTCCTACTGCTCCCTGGCCCCTCCCTT |
| | CCTCTAGTCACCCCCTCCTCTTGCACAGCCTCAATGCCCCTGGAGTTCCCTAACCCTTCT |
| | CCCTCAGTCTAGCAGGAGTTCTTCTCCAGGCAATTCTCAATTTTTCTTTTGGGGCTCCTG |
| | ATCCACCCTGACCTGACACTCCCTGACCTCTCAGTCCCCTACccccaccaatccacggca |
| | gctctcaccgggcag (SEQ ID NO: 32) |
| Mouse | ccaataacaacacactgtcctcgtttattgactacctgctgcgtaccaagctttgaaagt |
| (+ strand) | actcattctttaacgggaagcaagggcttataattttaaggtagacgggacagtttggat |
| | ttaaataccacctcttagctaaattgtcttgagtctaagtgaaacatcatctcttaactg |
| | accttgatacccgcatttgcaggtccaccctggaggccagagataaggcagagggagctg |
| | cagagaggaagggtcaatcaacacaatctgtagcctgctaggagctaggggagtgggaac |
| | tgttcaggtcagagccctcttgcactcagcccggactgtcttcgcccactgggcagtctg |
| | ccgtccatgcccgtgcgtgcggaccgacgcctggactaaccggctccaaaagtactttga |
| | tgggcgttgctgtttccaggacccgtggcctcacttctgggggcccaatctctccttcct |
| | ctgggagtggtccactctggaggcctctccagaatccggtggctgttgctggctgaggac |
| | aaagagcagacagctcagcagagcctctctcatgtcttgggagctcagtcggcgggccag |
| | ccggcgcagcaagggcaggaggtgctgccgggccaggcgggcaacaggcagcagcaggcg |
| | ggacagcagcgatcgaagcaccatcgggagcaatggaacgggcatggcaggacctgcacC |
| | TGCAAAGGGAACCCGGGTTTTAGACTGTACCTCAGGCACGCACCTCACCTGGCAAAGCAG |
| | GGTGCGGGGGTGTGGAGTCCTCCCTTCAGCTTATACctctgtactccccagttttcaact |
| | ggattagttttacagtgcagctttcttctggcatgaaagctggttaaggagttcactcac |
| | tgttatcacagatgggaagggagcccagggctggaaggtggtggggactGAGGCTAGGGC |
| | CTTTTCCAGAACCCACTTCCTTTAATCCCTCCCTCCCTTTGCATACTCTGACctgaagcc |
| | tgaacttcttgccctcctgctcaccagttctaaccggccagtggcagctctcaccagtca |
| | gaactgctcagaatcaatttcaggatgcttttgcctgcggtggattcagcatcact |
| | (SEQ ID NO: 33) |
| Mouse (− | agtgatgctgaatccaccgcaggcaaaagcatcctgaaattgattctgagcagttctgac |
| strand, reverse | tggtgagagctgccactggccggttagaactggtgagcaggagggcaagaagttcaggct |
| complement) - | tcagGTCAGAGTATGCAAAGGGAGGGAGGGATTAAAGGAAGTGGGTTCTGGAAAAGGCCC |
| start codon is | TAGCCTCagtccccaccaccttccagccctgggctcccttcccatctgtgataacagtga |
| bold & | gtgaactccttaaccagctttcatgccagaagaaagctgcactgtaaaactaatccagtt |
| underlined; | gaaaactggggagtacagagGTATAAGCTGAAGGGAGGACTCCACACCCCCGCACCCTGC |
| stop codon is | TTTGCCAGGTGAGGTGCGTGCCTGAGGTACAGTCTAAAACCCGGGTTCCCTTTGCAGgtg |
| bold and | caggtcctgccatgcccgttccattgctcccgatggtgcttcgatcgctgctgtcccgcc |
| italicized | tgctgctgcctgttgcccgcctggcccggcagcacctcctgcccttgctgcgccggctgg |
| | cccgccgactgagctcccaagacatgagagaggctctgctgagctgtctgctctttgtcc |
| | tcagccagcaacagccaccggattctggagaggcctccagagtggaccactcccagagga |
| | aggagagattgggcccccagaag [symbol] ggccacgggtcctggaaacagcaacgcccatcaa |
| | agtacttttggagccggttagtccaggcgtcggtccgcacgcacgggcatggacggcaga |
| | ctgcccagtgggcgaagacagtccgggctgagtgcaagagggctctgacctgaacagttc |
| | ccactcccctagctcctagcaggctacagattgtgttgattgacccttcctctctgcagc |
| | tccctctgccttatctctggcctccagggtggacctgcaaatgcgggtatcaaggtcagt |
| | taagagatgatgtttcacttagactcaagacaatttagctaagaggtggtatttaaatcc |
| | aaactgtcccgtctaccttaaaattataagcccttgcttcccgttaaagaatgagtactt |
| | tcaaagcttggtacgcagcaggtagtcaataaacgaggacagtgtgttgttattgg |
| | (SEQ ID NO: 34) |

Nonlimiting examples of extracellular wt-myomerger polypeptides are found in Table 2D. In other embodiments, the extracellular myomerger polypeptide has at least one amino acid modification relative to an extracellular wt-myomerger polypeptide. An extracellular wt-myomerger polypeptide can, in some embodiments, be an extracellular myomerger polypeptide from any animal including but not limited to a mammal, a rat, a cat, a rabbit, a human, a cow, a chicken, a turkey, a monkey, a tree shrew, a dog, a pig, a shrew, an elephant, or an opossum. Related nucleic acid molecules (e.g., cDNA or genomic DNA) can be any suitable nucleic acid molecule including but not limited to those made from appropriate changes (e.g., deletions or codon changes to make the same amino acid) to the nucleic acid molecules in Tables 2B and 2C.

TABLE 2D
Extracellular Myomerger

| Source | Polypeptide sequences |
|---|---|
| Mouse | RQHLLPLLRRLARRLSSQDMREALLSCLLFVLSQQQPPDS |
| | GEASRVDHSQRKERLGPQK (SEQ ID NO: 35) |
| Human | RQYLLPLLRRLARRLGSQDMREALLGCLLFILSQRHSPDA |
| | GEASRVDRLERRERLGPQK (SEQ ID NO: 36) |
| Cat | RRHLLPLLRRLARRLGSQDVREALLGCLLFILSQSRPPDA |
| | EEVSRVAGQERRERLAPPK (SEQ ID NO: 37) |
| Rabbit | RRHLLPLLRRLAQRLGSQGTREALLGCLLFVLSQRQPPDA |
| | SGEASRVDPPERKERLGRQK (SEQ ID NO: 38) |
| Dog | RRHLLPLLRGLARRLGSQEVREALLGCLLFILSQRHPPDA |
| | EEASRVAGQERKERLAPPK (SEQ ID NO: 39) |
| Elephant | RQHLLPLLRRLARRLGSQDMRQALLGCLLFVLSQQHPPDA |
| | GEASREALSERRGRLAPQK (SEQ ID NO: 40) |

One or more modifications, in some instances, can include an insertion, a deletion, a substitution, or combinations thereof. In certain embodiments, one or more modifications to a wt-myomerger polypeptide or extracellular wt-myomerger polypeptide can comprise an insertion, such, but not limited to an insertion at the C-terminus or at the N-terminus of the wt-myomerger polypeptide or extracellular wt-myomerger polypeptide. In some examples of the embodiments, an insertion can include (e.g., at the C-terminus, at the N-terminus, or at another place in the polypeptide) about 1, about 2, about 3, about 4, about 5, about 6, about 7, about 8, about 9, about 10, about 11, about 12, about 13, about 14, about 15, about 16, about 17, about 18, about 19, or about 20 amino acids (e.g., natural amino acids, or modified or unusual amino acids).

In some embodiments, the polypeptide does not encompass one or more naturally occurring polypeptides (e.g., does not encompass one or more of the wt-myomerger polypeptides). In other embodiments, the polypeptide does not encompass any of the wt-myomerger polypeptides. In other embodiments, the polypeptide does not encompass any of the extracellular wt-myomerger polypeptide. In some embodiments, the polypeptide does not encompass any naturally occurring polypeptide (e.g., does not encompass any of the wt-myomerger polypeptides or any other naturally occurring polypeptide).

In some embodiments, one or more modifications to a wt-myomerger polypeptide can include one or more substitutions, one or more insertions, or one or more deletions (or combinations thereof) to one or more amino acids in a hydrophobic region of a wt-myomerger polypeptide, in a signal region of a wt-myomerger polypeptide, in a transmembrane region of a wt-myomerger polypeptide, or in a combination thereof. In some embodiments, one or more modifications to a wt-myomerger polypeptide can include one or more substitutions or one or more deletions (or combinations thereof) to one or more amino acids in a hydrophobic region of a wt-myomerger polypeptide, in a signal region of a wt-myomerger polypeptide, in a transmembrane region of a wt-myomerger polypeptide, or in a combination thereof.

In some embodiments, the myomerger polypeptide can have a polypeptide sequence with an amino acid sequence identity to a wt-myomerger polypeptide (e.g., SEQ ID NO:17, SEQ ID NO:18, SEQ ID NO:19, SEQ ID NO:20, SEQ ID NO:21, SEQ ID NO:22, or SEQ ID NO:23) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. In some embodiments, the myomerger polypeptide sequence has an amino acid sequence identity to SEQ ID NO:17, SEQ ID NO:18, SEQ ID NO:19, SEQ ID NO:20, SEQ ID NO:21, SEQ ID NO:22, or SEQ ID NO:23 of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. The amino acid sequence identity (e.g., percent identity) can be determined by any suitable method, such as using BLAST, BLAST-2, ALIGN, ALIGN-2, Clustal Omega, or Megalign software. Unless otherwise indicated, the amino acid sequence identity (e.g., percent identity) is determined using BLAST-2.

In some embodiments, the myomerger polypeptide has (e.g., as compared to a wt-myomerger polypeptide or as compared to the absence of a myomerger polypeptide) an increased ability to permeabilize membranes, an increased ability to form pores in membranes, an increase in the ability to stress membranes, an increase in the ability to lyse cells or liposomes, an increased ability to activate fusion, a decreased ability to activate fusion, an increased ability to confer fusogenicity, a decreased ability to confer fusogenicity, an increased level of expression during embryonic development, a decreased level of expression during embryonic development, an increased level of expression during myogenesis in adult organisms (e.g., older than embryonic), a decreased level of expression during myogenesis in adult organisms (e.g., older than embryonic), an increased level of induction of myogenesis in adult organisms (e.g., older than embryonic), a decreased of induction of myogenesis in adult organisms (e.g., older than embryonic), an increased affinity for membranes, a decreased affinity for membranes, an increased level of association with membrane compartment, a decreased level association with membrane compartment, or combinations thereof. In other embodiments, the myomerger polypeptide has (e.g., as compared to a wt-myomerger polypeptide or as compared to the absence of a myomerger polypeptide) an increased ability to permeabilize membranes, an increased ability to form pores in membranes, an increase in the ability to stress membranes, an increase in the ability to lyse cells or liposomes, an increased ability to activate fusion, an increased ability to confer fusogenicity, an increased level of expression during embryonic development, an increased level of expression during myogenesis in adult organisms (e.g., older than embryonic), an increased level of induction of myogenesis in adult organisms (e.g., older than embryonic), an increased affinity for membranes, an increased level of association with membrane compartment, or combinations thereof.

Some embodiments of the invention include nucleic acid molecules that can encode for the myomerger polypeptide (“myomerger nucleic acid molecules”). In certain embodiments, the myomerger nucleic acid molecule is included in a vector (e.g., a viral vector, a retroviral vector, a lentiviral vector, a VSV vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, or a nonconjugative vector). In certain embodiments, the myomerger nucleic acid molecule is in a cell, such as an insect cell (e.g., an Sf9 cell) or a mammalian cell (e.g., a human cell, a rat cell a mouse cell, a muscle cell, a non-muscle cell, a myoblast, a fibroblast, a C2C12 cell, a 10T ½ fibroblast, an NIH/3T3 cell, a CHO cell, a mesenchymal stem cell (MSC), a hematopoietic stem cell, a blood cell, a bone marrow cell, or an adipose stem cell).

In other embodiments, the myomerger nucleic acid molecule comprises one or more nucleic acid sequences that are not used to encode for the polypeptide (e.g., one or more introns). For example, the myomerger nucleic acid molecule can comprise a nucleic acid sequence as found in nature (e.g., including introns). In certain embodiments, the myomerger nucleic acid molecule differs from the one or more nucleic acid molecules in nature because the myomerger nucleic acid molecule does not include one or more introns. In some embodiments, the myomerger nucleic acid molecule is a cDNA molecule (“myomerger cDNA molecule”). In certain embodiments, the myomerger cDNA molecule is identical to a nucleic acid molecule found in nature. In other embodiments, the myomerger cDNA molecule is not identical to a nucleic acid molecule found in nature (e.g., due to the myomerger cDNA molecule not including one or more introns in the nucleic acid molecule found in nature).

In some embodiments, the myomerger nucleic acid molecule sequence has a sequence identity to a nucleic acid molecule encoding a wt-myomerger polypeptide (e.g., SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34) of about 60%, about 70%, about 80%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 60%, at least about 70%, at least about 80%, at least about 90%, at least about 95%, at least about 99%, or at least about 99.5%. In some embodiments, the myomerger nucleic acid molecule sequence has a sequence identity to SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34 of about 60%, about 70%, about 80%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 60%, at least about 70%, at least about 80%, at least about 90%, at least about 95%, at least about 99%, or at least about 99.5%. Nonlimiting examples of wt-myomerger polypeptides and wt-myomerger nucleic acid molecules can be found in Table 2. The nucleic acid sequence identity (e.g., percent identity) can be determined by any suitable method, such as using BLAST, BLAST-2, ALIGN, ALIGN-2, Clustal Omega, or Megalign software. Unless otherwise indicated, the nucleic acid sequence identity (e.g., percent identity) is determined using BLAST-2.

In some embodiments, the myomerger nucleic acid molecule encodes for a polypeptide that has one or more modifications to wt-myomerger polypeptide in a hydrophobic region, in a signal region, in a transmembrane region, or in a combination thereof.

The myomerger nucleic acid molecule can be made using any suitable technique, such as but not limited to, chemical synthesis, enzymatic production or biological production. Chemical synthesis of a nucleic acid molecule can include, for example, a nucleic acid molecule made by in vitro chemical synthesis using phosphotriester, phosphite or phosphoramidite chemistry and solid phase techniques, or via deoxynucleoside H-phosphonate intermediates. Enzymatically produced nucleic acid molecules can be accomplished using any suitable method including but not limited to Polymerase Chain Reaction (PCR). Biologically produced nucleic acid molecules can be accomplished using any suitable method including but not limited to a recombinant nucleic acid produced (i.e., replicated) in a living cell, such as a recombinant DNA vector replicated in bacteria.

In some embodiments, one or more modifications to an extracellular wt-myomerger polypeptide can include one or more substitutions, one or more insertions, or one or more deletions (or combinations thereof) to one or more amino acids in a hydrophobic region of an extracellular wt-myomerger polypeptide, in a signal region of an extracellular wt-myomerger polypeptide, or in a combination thereof. In some embodiments, one or more modifications to an extracellular wt-myomerger polypeptide can include one or more substitutions or one or more deletions (or combinations thereof) to one or more amino acids in a hydrophobic region of an extracellular wt-myomerger polypeptide, in a signal region of an extracellular wt-myomerger polypeptide, or in a combination thereof.

In other embodiments, the extracellular myomerger polypeptide comprises (a) amino acids 4-15 of any of SEQ ID Nos: 35-40, (b) amino acids 18-32 of any of SEQ ID Nos: 35-40, or (c) both. In other embodiments, the extracellular myomerger polypeptide comprises (a) LLPLLRRLARRL (SEQ ID NO:41), (b) QDMREALLSCLLFVL (SEQ ID NO:42) or QDMREALLGCLLFIL (SEQ ID NO:43), or (c) both.

In some embodiments, the extracellular myomerger polypeptide can have a polypeptide sequence with an amino acid sequence identity to an extracellular wt-myomerger polypeptide (e.g., SEQ ID NO:35, SEQ ID NO:36, SEQ ID NO:37, SEQ ID NO:38, SEQ ID NO:39, and SEQ ID NO:40) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. In some embodiments, the extracellular myomerger polypeptide sequence has an amino acid sequence identity to SEQ ID NO:35, SEQ ID NO:36, SEQ ID NO:37, SEQ ID NO:38, SEQ ID NO:39, and SEQ ID NO:40 of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. The amino acid sequence identity (e.g., percent identity) can be determined by any suitable method, such as using BLAST, BLAST-2, ALIGN, ALIGN-2, Clustal Omega, or Megalign software. Unless otherwise indicated, the amino acid sequence identity (e.g., percent identity) is determined using BLAST-2.

In some embodiments, the extracellular myomerger polypeptide has (e.g., as compared to a wt-myomerger polypeptide or an extracellular wt-myomerger polypeptide, or as compared to the absence of a myomerger polypeptide or an extracellular wt-myomerger polypeptide) an increased ability to permeabilize membranes, an increased ability to form pores in membranes, an increase in the ability to stress membranes, an increase in the ability to lyse cells or liposomes, an increased ability to activate fusion, a decreased ability to activate fusion, an increased ability to confer fusogenicity, a decreased ability to confer fusogenicity, an increased level of expression during embryonic development, a decreased level of expression during embryonic development, an increased level of expression during myogenesis in adult organisms (e.g., older than embryonic), a decreased level of expression during myogenesis in adult organisms (e.g., older than embryonic), an increased level of induction of myogenesis in adult organisms (e.g., older than embryonic), a decreased of induction of myogenesis in adult organisms (e.g., older than embryonic), an increased affinity for membranes, a decreased affinity for membranes, an increased level of association with membrane compartment, a decreased level association with membrane compartment, or combinations thereof. In other embodiments, the extracellular myomerger polypeptide has (e.g., as compared to a wt-myomerger polypeptide or an extracellular wt-myomerger polypeptide, or as compared to the absence of a myomerger polypeptide or an extracellular myomerger polypeptide) an increased ability to permeabilize membranes, an increased ability to form pores in membranes, an increase in the ability to stress membranes, an increase in the ability to lyse cells or liposomes, an increased ability to activate fusion, an increased ability to confer fusogenicity, an increased level of expression during embryonic development, an increased level of expression during myogenesis in adult organisms (e.g., older than embryonic), an increased level of induction of myogenesis in adult organisms (e.g., older than embryonic), an increased affinity for membranes, an increased level of association with membrane compartment, or combinations thereof.

Some embodiments of the invention include nucleic acid molecules that can encode for an extracellular myomerger polypeptide (“extracellular myomerger nucleic acid molecules”). In certain embodiments, the extracellular myomerger nucleic acid molecule is included in a vector (e.g., a viral vector, a retroviral vector, a lentiviral vector, a VSV vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, or a nonconjugative vector). In certain embodiments, the extracellular myomerger nucleic acid molecule is in a cell, such as an insect cell (e.g., an Sf9 cell) or a mammalian cell (e.g., a human cell, a rat cell a mouse cell, a muscle cell, a non-muscle cell, a myoblast, a fibroblast, a C2C12 cell, a 10T ½ fibroblast, an NIH/3T3 cell, a CHO cell, a mesenchymal stem cell (MSC), a hematopoietic stem cell, a blood cell, a bone marrow cell, or an adipose stem cell).

In other embodiments, the extracellular myomerger nucleic acid molecule comprises one or more nucleic acid sequences that are not used to encode for the polypeptide (e.g., one or more introns). For example, the extracellular myomerger nucleic acid molecule can comprise a nucleic acid sequence as found in nature (e.g., including introns). In certain embodiments, the extracellular myomerger nucleic acid molecule differs from the one or more nucleic acid molecules in nature because the extracellular myomerger nucleic acid molecule does not include one or more introns. In some embodiments, the extracellular myomerger nucleic acid molecule is a cDNA molecule (“myomerger cDNA molecule”). In certain embodiments, the extracellular myomerger cDNA molecule is identical to a nucleic acid molecule found in nature. In other embodiments, the extracellular myomerger cDNA molecule is not identical to a nucleic acid molecule found in nature (e.g., due to the myomerger cDNA molecule not including one or more introns in the nucleic acid molecule found in nature).

In some embodiments, the extracellular myomerger nucleic acid molecule sequence has a sequence identity to a nucleic acid molecule encoding an extracellular wt-myomerger polypeptide (e.g., see Table 2B and 2C, which can include changes to provide appropriate cDNA sequences or equivalent genomic-like sequences) of about 60%, about 70%, about 80%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 60%, at least about 70%, at least about 80%, at least about 90%, at least about 95%, at least about 99%, or at least about 99.5%. Nonlimiting examples of extracellular wt-myomerger polypeptides and wt-myomerger nucleic acid molecules can be found in Table 2. The nucleic acid sequence identity (e.g., percent identity) can be determined by any suitable method, such as using BLAST, BLAST-2, ALIGN, ALIGN-2, Clustal Omega, or Megalign software. Unless otherwise indicated, the nucleic acid sequence identity (e.g., percent identity) is determined using BLAST-2.

In some embodiments, the extracellular myomerger nucleic acid molecule encodes for a polypeptide that has one or more modifications to extracellular wt-myomerger polypeptide in a hydrophobic region, in a signal region, or in a combination thereof.

The extracellular myomerger nucleic acid molecule can be made using any suitable technique, such as but not limited to, chemical synthesis, enzymatic production or biological production. Chemical synthesis of a nucleic acid molecule can include, for example, a nucleic acid molecule made by in vitro chemical synthesis using phosphotriester, phosphite or phosphoramidite chemistry and solid phase techniques, or via deoxynucleoside H-phosphonate intermediates. Enzymatically produced nucleic acid molecules can be accomplished using any suitable method including but not limited to Polymerase Chain Reaction (PCR). Biologically produced nucleic acid molecules can be accomplished using any suitable method including but not limited to a recombinant nucleic acid produced (i.e., replicated) in a living cell, such as a recombinant DNA vector replicated in bacteria.

Modifications or changes made in the structure of the nucleic acid molecules and/or polypeptides can be used in the present invention. In certain embodiments, a polypeptide can be modified (e.g., by one or more insertions, one or more deletions, or one or more substitutions (e.g., conservative substitutions)). In some embodiments, the polypeptide which was modified does not have an appreciable loss (e.g., a decrease in a function of less than about 1%, less than about 5%, less than about 10%, less than about 25%, less than about 50%, less than about 75%, less than about 90%, less than about 95%, less than about 99%, or less than about 100%) of one or more chosen functions of the unmodified polypeptide such as, for example, the ability to permeabilize membranes, the ability to form pores in membranes, the ability to stress membranes, the ability to lyse cells or liposomes, the ability to make changes to the cytoskeleton of the cell (e.g., reorganizing the cytoskeleton, rearranging the cytoskeleton, making changes to the cytoskeleton to allow the cell to fuse), the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the polypeptide which was modified retains desired levels (e.g., at least about 20%, at least about 40%, at least about 50%, at least about 75%, at least about 80%, at least about 90%, at least about 95%, or at least about 99%) of one or more functions of the unmodified polypeptide, such as, for example, the ability to permeabilize membranes, the ability to form pores in membranes, the ability to stress membranes, the ability to lyse cells or liposomes, the ability to make changes to the cytoskeleton of the cell (e.g., reorganizing the cytoskeleton, rearranging the cytoskeleton, making changes to the cytoskeleton to allow the cell to fuse), the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the polypeptide after modification has an increased level of one or more functions as compared to the unmodified polypeptide. Nucleic acid molecules can be designed to encode for such a modified polypeptide, and such nucleic acid molecules are encompassed by the present invention.

A “functional polypeptide” is defined as a polypeptide (e.g., a myomerger polypeptide, an extracellular myomerger polypeptide, or a modified extracellular myomerger polypeptide) that has desired levels (e.g., at least about 20%, at least about 40%, at least about 50%, at least about 75%, at least about 80%, at least about 90%, at least about 95%, or at least about 99%, as compared to another polypeptide, such as a naturally occurring polypeptide) of one or more functions such as, for example, the ability to increase permeability of membranes, the ability to form pores in membranes, the ability to stress membranes, the ability to lyse cells or liposomes, the ability to make changes to the cytoskeleton of the cell (e.g., reorganizing the cytoskeleton, rearranging the cytoskeleton, making changes to the cytoskeleton to allow the cell to fuse), the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the function polypeptide has an increased level of one or more functions as compared to another polypeptide (e.g., a naturally occurring polypeptide). Nucleic acid molecules can be designed to encode for functional polypeptides, and such nucleic acid molecules are encompassed by the present invention.

A “functionally equivalent” polypeptide (e.g., a modified myomerger polypeptide or a modified extracellular myomerger polypeptide) is defined as a polypeptide that has been modified (e.g., by one or more insertions, one or more deletions, or one or more substitutions (e.g., conservative substitutions)) from an original polypeptide (e.g., a wt-myomerger polypeptide) and that modified polypeptide retains desired levels (e.g., at least about 20%, at least about 40%, at least about 50%, at least about 75%, at least about 80%, at least about 90%, at least about 95%, or at least about 99%) of one or more functions of the original polypeptide, such as, for example, the ability to increase permeability of membranes, the ability to form pores in membranes, the ability to stress membranes, the ability to lyse cells or liposomes, the ability to make changes to the cytoskeleton of the cell (e.g., reorganizing the cytoskeleton, rearranging the cytoskeleton, making changes to the cytoskeleton to allow the cell to fuse), the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the functionally equivalent polypeptide has an increased level of one or more functions compared to the original polypeptide. Nucleic acid molecules can be designed to encode for functionally equivalent polypeptides, and such nucleic acid molecules are encompassed by the present invention.

In certain embodiments, the shorter the length of a polypeptide, the fewer the modifications (e.g., substitutions) that can be made within the polypeptide while retaining, for example, a desired level of a chosen function. In some instances, longer domains can have a greater number of such changes while retaining, for example, a desired level of a chosen function. In other embodiments, a full-length polypeptide can have more tolerance for a fixed number of changes while retaining, for example, a desired level of a chosen function, as compared to a shorter length of that polypeptide.

The design of substitutions can take many forms, including but not limited to those described herein. In some embodiments, the hydropathic index of amino acids may be considered in designing substitutions. In the hydropathic index, each amino acid is assigned a hydropathic index on the basis of their hydrophobicity or charge characteristics, as follows: isoleucine (+4.5); valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); or arginine (−4.5). In some instances, certain amino acids may be substituted for other amino acids having a similar hydropathic index. In making changes based upon the hydropathic index, the substitution of amino acids with hydropathic indices can be made with amino acids that have an index difference of no more than ±2, no more than ±1, or no more than ±0.5.

In some embodiments, substitutions can also be made based on hydrophilicity values. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids with hydrophilicity values can be made with amino acids that have a value of no more than ±2, no more than ±1, or no more than ±0.5.

A “conservative substitution” in an amino acid sequence or polypeptide indicates that a given amino acid residue is replaced by a residue having similar physiochemical characteristics (e.g., no more than ±1 when based on hydropathic index or no more than ±1 when base on hydrophilicity values). Examples of conservative substitutions include (a) substitution of one aliphatic residue for another with an aliphatic residue, (b) substitution of one of Ile, Val, Leu, or Ala for one another of Ile, Val, Leu, or Ala, (c) substitution of one of Gly, Ile, Val, Leu, or Ala for one another of Gly, Ile, Val, Leu, or Ala, (d) substitution of one polar residue for another polar residue, (e) substitution of one of Lys and Arg with another of Lys and Arg, (f) substitution of one of Glu and Asp with another of Glu and Asp, (g) substitution of one of Gln and Asn with another of Gln and Asn, (h) substitution of one hydroxyl or sulfur containing residue with another hydroxyl or sulfur containing residue, (i) substitution of one of Ser, Cys, Thr, or Met with another of Ser, Cys, Thr, or Met, (j) substitution of one aromatic residue for another with an aromatic residue, (k) substitution of one of Phe, Tyr, or Trp with another of Phe, Tyr, or Trp, (1) substitution of one basic residue for another basic residue, (m) substitution of one of His, Lys, or Arg with another of His, Lys, or Arg, (n) substitution of an acidic/amide residue with another acidic/amide residue, (o) substitution of one of Asp, Glu, Asn, or Gln with another of Asp, Glu, Asn, or Gln, (p) substitution of a residue with another residue of a similar size, and (q) substitution of one of Ala, Gly, or Ser with another of Ala, Gly, or Ser. In some embodiments, each amino acid in a hydrophobic region of a polypeptide can be substituted with conservative substitutions (e.g., any combination of conservative substitutions relating to hydrophobic residues).

While discussion has focused on amino acid changes, it will be appreciated that these changes may occur by alteration of the encoding DNA; taking into consideration also that the genetic code is degenerate and that two or more codons may code for the same amino acid. A table of amino acids and their codons is presented below for use in such embodiments, as well as for other uses, such as in the design of probes and primers and the like.

Tables A2 and B2. Amino Acid Designations and Codon Table

| Table A2 - Amino Acid | | | Table B2 - Codons for |
|---|---|---|---|
| Designations | | | Amino Acids |
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The term “functionally equivalent codon” is used herein to refer to codons that encode the same amino acid, such as the six codons for arginine or serine.

In certain instances, the nucleic acid molecule can be engineered to contain distinct sequences while at the same time retaining the capacity to encode a desired polypeptide. In some embodiments, this can be accomplished owing to the degeneracy of the genetic code (i.e., the presence of multiple codons) which encode for the same amino acids. In other instances, it can be accomplished by including, adding, or excluding introns in the nucleic acid molecule.

In certain embodiments, a restriction enzyme recognition sequence can be introduced into a nucleic acid sequence while maintaining the ability of that nucleic acid molecule to encode a desired polypeptide. In other embodiments, a CRISPR system (e.g., a CRISPR system comprising one or more of guide RNA, crRNA, tracrRNA, sgRNA, DNA repair template, and Cas protein, such as but not limited to CRISPR/Cas9) can be used to introduce a nucleic acid molecule while maintaining the ability of that nucleic acid molecule to encode a desired polypeptide.

It will also be understood that amino acid sequences (e.g., polypeptides) and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, such as including the maintenance of biological activity where polypeptide expression is concerned. The addition of terminal sequences particularly applies to nucleic acid sequences that may, for example, include various non-coding sequences flanking either of the 5′ or 3′ portions of the coding region or may include various internal sequences, (i.e., introns) which can occur within genes.

Some embodiments of the present invention rely on or use synthesis of polypeptides in cyto, via transcription and translation of appropriate nucleic acid molecules (e.g., nucleic acid sequences as discussed herein). These polypeptides will include the twenty “natural” amino acids, and post-translational modifications thereof. In vitro peptide synthesis permits the use of modified or unusual amino acids. In some embodiments, the polypeptide encompasses modifications (e.g., one or more substitutions or one or more insertions) that include one or more modified or unusual amino acids. A table of exemplary, but not limiting, modified or unusual amino acids is provided in Table C2.

TABLE C2
Modified or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, | AHyl | allo-Hydroxylysine |
| | beta-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

The presently disclosed subject matter further includes a method of producing a polypeptide (e.g., a myomerger polypeptide or an extracellular myomerger polypeptide). Any suitable method can used to make the polypeptides including but not limited to expression through any suitable molecular biological technique (e.g., using a prokaryotic or eukaryotic expression system), isolation from a source in nature, or chemical synthesis. Eukaryotic expression systems include plant-based systems; insect cell systems via recombinant baculoviruses; whole insect systems via recombinant baculoviruses; genetically engineered yeast systems, including but not limited to Saccharomyces sp. and Pichia spp.; and mammalian cell systems, including but not limited to C2C12 cells, 10T ½ fibroblasts, NIH/3T3 fibroblasts, mesenchymal stem cells (MSCs), hematopoietic stem cells, Chinese hamster ovary cells or other cell lines commonly used for industrial scale expression of recombinant proteins. In some embodiments, useful plant-based expression systems can include transgenic plant systems. In some embodiments, useful plant-based expression systems can include transplastomic plant systems.

In some embodiments, a method of producing the polypeptide includes providing a host cell comprising a nucleic acid molecule, as disclosed herein, operatively linked to a promoter operable under conditions whereby the encoded polypeptide is expressed; and recovering the polypeptide from the host cell.

Pseudotyped Particles

Some embodiments of the invention include pseudotyped particles, such as but not limited to pseudotyped exosomes and pseudotyped viruses (e.g., pseudotyped Vesicular Stomatitis Virus (VSV) and pseudotyped lentiviruses). In certain embodiments, one or more myomaker polypeptides, one or more myomerger polypeptides, or a combination thereof is present on the surface of the pseudotyped particle. In certain embodiments, one or more myomaker polypeptides, one or more myomerger polypeptides, or a combination thereof is present on the lipid envelope of the pseudotyped particle. In other embodiments, “pseudotyped” means (a) that the particle has one or more polypeptides (e.g., one or more myomaker polypeptides, one or more myomerger polypeptides, or a combination thereof) on the pseudotyped particle's surface (e.g., exosome surface or virus envelop) that are not found in the corresponding naturally occurring particle, (b) that the particle has a larger amount of one or more polypeptides (e.g., a myomaker polypeptide, a myomerger polypeptide, or both) on the pseudotyped particle's surface than that found in the corresponding naturally occurring particle, (c) that the particle has a larger amount of one or more polypeptides (e.g., a myomaker polypeptide, a myomerger polypeptide, or both) in the pseudotyped particle (i.e., by measuring the total amount of polypeptide in the particle) than that found in the corresponding naturally occurring particle, or (d) a combination of (a), (b), or (c). In some embodiments, (a) the pseudotyped particle has one or more polypeptides on the pseudotyped particle's surface that are not found in the corresponding non-pseudotyped particle, (b) the pseudotyped particle has a larger amount of one or more polypeptides on the pseudotyped particle's surface than that found in the corresponding non-pseudotyped particle, (c) the pseudotyped particle has a larger amount of one or more polypeptides in the pseudotyped particle, by measuring the total amount of polypeptide in the pseudotyped particle, than that found in the corresponding non-pseudotyped particle, or (d) a combination of (a), (b), or (c).

In some embodiments of the pseudotyped particle, the one or more myomerger polypeptides comprise a human myomerger polypeptide (e.g., SEQ ID NO:19). In other embodiments of the pseudotyped particle, the one or more myomaker polypeptides comprise a human myomaker polypeptide (e.g., SEQ ID NO:1).

In certain embodiments, the pseudotyped particle has a size of 20-500 nm, 30-150 nm, or 80-120 nm.

In other embodiments, the pseudotyped particle comprises a nucleic acid encoding a gene of interest or a nucleic acid that can modulate gene expression.

In other embodiments, the pseudotyped particle comprises a nucleic acid encoding a gene of interest (e.g., a therapeutic gene, a reporter gene, a dystrophin nucleic acid molecule, a microdystrophin nucleic acid molecule, a minidystrophin nucleic acid molecule, a gene that is mutated in a genetic muscle disease (e.g., dystrophin, titin, or skeletal muscle alpha-actin), a gene for a secreted therapeutic factor, green fluorescent protein (GFP) or tdTomato). In yet other embodiments, the gene of interest does not encode for a myomaker protein or myomerger protein. In still other embodiments, the gene of interest is selected from the group consisting of a therapeutic gene (e.g., a gene that encodes a dystrophin polypeptide) or a reporter gene (e.g., GFP or tdTomato). In some embodiments, the gene of interest is a gene for delivery to a muscle cell.

In other embodiments, the pseudotyped particle comprises a nucleic acid that can modulate (e.g., increase or decrease) gene expression (e.g., gRNA/Cas, gRNA/Cas9 or anti-sense oligonucleotides). In other embodiments, the pseudotyped particle comprises a nucleic acid that can decrease gene expression (e.g., gRNA/Cas, gRNA/Cas9 or anti-sense oligonucleotides). In certain embodiments, the nucleic acid that can modulate gene expression is gRNA/Cas (e.g., gRNA/Cas9), which is guide RNA (gRNA) and a CRISPR-associated endonuclease (Cas protein, such as Cas9). In still other embodiments, the gRNA can be a short synthetic RNA composed of a scaffold sequence for Cas-binding and, in certain embodiments, a user-defined 5-60 nucleotide spacer can define the genomic target to be modified. In some embodiments, one can change the genomic target of the Cas protein (e.g., Cas9 protein) by changing the target sequence present in the gRNA. In yet other embodiments, the anti-sense oligonucleotide can be synthetic DNA oligomers or synthetic RNA oligomers that hybridize to a target RNA in a sequence-specific manner. In certain embodiments, the anti-sense oligonucleotide can inhibit gene expression, modulate splicing of a precursor messenger RNA, and/or inactivate microRNAs. In certain embodiments, the length of an anti-sense oligonucleotide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, from 5 to 60, 10 to 50, 15 to 40, or 15 to 30 nucleotides long.

In certain embodiments, the pseudotyped particle further comprises one or more nucleic acid molecules and the one or more nucleic acid molecules comprises one or more myomaker nucleic acid molecules. In other embodiments, the pseudotyped particle further comprises one or more nucleic acid molecules and the one or more nucleic acid molecules comprises one or more myomerger nucleic acid molecules. In some embodiments, the pseudotyped particle further comprises one or more nucleic acid molecules and the one or more nucleic acid molecules comprises one or more myomerger nucleic acid molecules and one or more myomaker nucleic acid molecules.

In some embodiments, the pseudotyped particle exhibits fusogenic activity with a target cell upon binding of the myomaker polypeptide and/or the myomerger polypeptide to a myomaker polypeptide and/or myomerger polypeptide on the target cell. In other embodiments, the target cell endogenously expresses a myomaker polypeptide and/or myomerger polypeptide, optionally wherein the target cell is a muscle cell. In certain embodiments, the target cell does not endogenously express a myomaker polypeptide and/or a myomerger polypeptide, optionally wherein the target cell is a non-muscle cell. In other embodiments, the target cell can be but is not limited an animal cell, a vertebrate cell, a mammalian cell, a human cell, a rat cell, a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In yet other embodiments, the target cell can be but is not limited myoblasts (e.g., proliferative myoblasts), myotubes (e.g., differentiating myotube or differentiated myotubes), and fibroblasts. In still other embodiments, the target cell is a fibroblast, a mesenchymal stem cell (MSC), a hematopoietic stem cell, a blood cell, a bone marrow cell, or an adipose stem cell.

Some embodiments of the invention include pseudotyped particles, such as but not limited to pseudotyped exosomes (e.g., as described herein), pseudotyped VSV (e.g., as described herein) and pseudotyped lentiviruses (e.g., as described herein).

Pseudotyped Exosomes

Some embodiments of the invention include pseudotyped exosomes, as disclosed herein. In certain embodiments, the pseudotyped exosome surface comprises one or more myomerger polypeptides, one or more myomaker polypeptides, or a combination thereof. In certain embodiments, one or more myomaker polypeptides, one or more myomerger polypeptides, or a combination thereof is present on the surface of the pseudotyped exosomes. In certain embodiments, one or more myomaker polypeptides, one or more myomerger polypeptides, or a combination thereof is present on the lipid envelope of the pseudotyped exosomes.

In some embodiments of the pseudotyped exosome, the one or more myomerger polypeptides comprise a human myomerger polypeptide (e.g., SEQ ID NO:19). In other embodiments of the pseudotyped exosome, the one or more myomaker polypeptides comprise a human myomaker polypeptide (e.g., SEQ ID NO:1).

In other embodiments, the amount (e.g., the total amount in the pseudotyped exosome and/or the amount on the surface of the pseudotyped exosome) of at least one of the myomerger polypeptides or the myomaker polypeptides, is greater than the amount of that same polypeptide as compared to a naturally occurring exosome (e.g., a naturally occurring exosome that comprises one or more myomerger polypeptides, one or more myomaker polypeptides, or a combination thereof). In yet other embodiments, the amount is greater by about 5%, about 10%, about 15% about 20%, about 25%, about 30%, about 40%, about 50%, about 60%, about 75%, at least about 5%, at least about 10%, at least about 20%, at least about 25%, at least about 50%, or at least about 75%. In certain embodiments, the naturally occurring exosome can be produced by a muscle cell, a myoblast (e.g., myotube), or a mesenchymal stem cell.

The total amount of myomaker polypeptide and/or myomerger polypeptide in the pseudotyped exosome or the amount of myomaker polypeptide and/or myomerger polypeptide on the surface of the pseudotyped exosome can be measured using any suitable method including but not limited to one or more of western blot, cell sorting or mass spectrometry. Unless otherwise indicated the total amount of myomaker polypeptide and/or myomerger polypeptide in the pseudotyped exosome or the amount of myomaker polypeptide and/or myomerger polypeptide on the surface of the pseudotyped exosome is measured using western blot.

In some embodiments, the pseudotyped exosomes increase uptake of exosomes in certain cells (e.g., myogenic cells, such as but not limited to myoblasts (e.g., proliferative myoblasts), myotubes (e.g., differentiating myotube or differentiated myotubes)) compared to naturally occurring exosomes (e.g., a naturally occurring exosome that comprises one or more myomerger polypeptides, one or more myomaker polypeptides, or a combination thereof) or exosomes that do not comprise a myomerger polypeptide, a myomaker polypeptide, or both). In certain embodiments, the increased uptake is greater by about 5%, about 10%, about 15% about 20%, about 25%, about 30%, about 40%, about 50%, about 60%, about 75%, at least about 5%, at least about 10%, at least about 20%, at least about 25%, at least about 50%, or at least about 75%.

In still other embodiments, the pseudotyped exosomes can be produced using any suitable exosome producing cell. In yet other embodiments, the exosome producing cell does not produce exosomes comprising one or more myomerger polypeptides, one or more myomaker polypeptides, or a combination thereof, in its naturally occurring state. In other embodiments, the exosome producing cell expresses or overexpresses one or more myomerger polypeptides, one or more myomaker polypeptides, or a combination thereof. In other embodiments, the exosome producing cell can be but is not limited myoblasts (e.g., proliferative myoblasts), myotubes (e.g., differentiating myotube or differentiated myotubes), and fibroblasts. In yet other embodiments, the pseudotyped exosomes can be produced by cells that have been altered to express (e.g., fibroblast cells, BHK21 cells, HEK293t cells, or 10T½ cells) one or more myomerger polypeptides, one or more myomaker polypeptides, or a combination thereof. In certain embodiments, the pseudotyped exosomes can be produced by cells that have been altered to overexpress (e.g., C2C12 cells, fibroblast cells, BHK21 cells, HEK293t cells, or 10T½ cells) one or more myomerger polypeptides, one or more myomaker polypeptides, or a combination thereof. In some embodiments, the exosome producing cell can be a modified cell (e.g., as disclosed herein).

The method for preparing the pseudotyped exosomes can include any suitable method, including those disclosed herein. In some embodiments, the method for preparing the pseudotyped exosomes comprises (a) growing exosome producing cells that express (e.g., or optionally overexpresses) at least one myomerger polypeptide, at least one myomaker polypeptide, or a combination thereof and (b) placing the exosome producing cells in exosome depleted media (e.g., exosome depleted fetal bovine serum or exosome depleted horse serum). In some embodiments, the method for producing a pseudotyped exosome comprising (a) growing exosome producing cells that express (e.g., or optionally overexpresses) at least one myomerger polypeptide, at least one myomaker polypeptide, a polypeptide of interest, or a combination thereof, (b) optionally contacting the exosome producing cells with a nucleic acid of interest (e.g., a dystrophin nucleic acid molecule, such as those disclosed herein), a polypeptide of interest (e.g., a dystrophin polypeptide, such as those disclosed herein), or a combination thereof, and (c) placing the exosome producing cells in an exosome depleted media (e.g., exosome depleted fetal bovine serum or exosome depleted horse serum). The contacting in step (b) can comprise any suitable method including but not limited to injection, microinjection, electroporation, sonication, calcium ion treatment, calcium phosphate precipitation, PEG-DMSO treatment, DE-Dextran treatment, liposome mediated transformation, or a receptor mediated transformation. In certain embodiments, the exosome producing cell overexpresses one or more myomerger polypeptides, one or more myomaker polypeptides, or a combination thereof. In still other embodiments, the exosome producing cell can be but is not limited an animal cell, a vertebrate cell, a mammalian cell, a human cell, a rat cell, a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In still other embodiments, the exosome producing cell can be but is not limited myoblasts (e.g., proliferative myoblasts), myotubes (e.g., differentiating myotube or differentiated myotubes), and fibroblasts. In yet other embodiments, the exosome producing cells have been altered to express (e.g., fibroblast cells, BHK21 cells, HEK293t cells, or 10T½ cells) one or more myomerger polypeptides, one or more myomaker polypeptides, or a combination thereof. In certain embodiments, the exosome producing cells have been altered to overexpress (e.g., C2C12 cells, fibroblast cells, BHK21 cells, HEK293t cells, or 10T½ cells) one or more myomerger polypeptides, one or more myomaker polypeptides, or a combination thereof. In some embodiments, the media in (a) is removed or partially (e.g., >80%) removed prior to step (c), using any suitable method including but not limited to centrifugation, filtration, ultracentrifugation, ultrafiltration, or chromatography. In certain embodiments, after step (c), the cells are separated or partially (e.g., >80%) separated from the supernatant using any suitable method including but not limited to centrifugation, filtration, ultracentrifugation, ultrafiltration, or chromatography.

In some embodiments, the exosomes can be recovered (e.g., after step (c)) using any suitable method including but not limited to centrifugation, filtration, ultracentrifugation, ultrafiltration, chromatography, or hydrophobic interaction chromatography (HIC) (e.g., using a polyester, capillary-channeled polymer (PET C-CP) stationary phase).

In some embodiments, the exosomes can be cryopreserved. In other embodiments, proteinase inhibitors can optionally be included in freezing media. Other optional additives to the freezing media can be used to enhance preservation of exosome biological activity, and can sometimes be similar to those used for cryopreservation of intact cells, such as but are not limited to DMSO, glycerol, polyethylene glycol, and combinations thereof.

In certain embodiments, the pseudotyped exosome has a size of 20-500 nm, 30-150 nm, or 80-120 nm.

In other embodiments, the pseudotyped exosome comprises a nucleic acid encoding a gene of interest or a nucleic acid that can modulate gene expression.

In other embodiments, the pseudotyped exosome comprises a nucleic acid encoding a gene of interest (e.g., a therapeutic gene, a reporter gene, a dystrophin nucleic acid molecule, a microdystrophin nucleic acid molecule, a minidystrophin nucleic acid molecule, a gene that is mutated in a genetic muscle disease (e.g., dystrophin, titin, or skeletal muscle alpha-actin), a gene for a secreted therapeutic factor, green fluorescent protein (GFP) or tdTomato). In yet other embodiments, the gene of interest does not encode for a myomaker protein or myomerger protein. In still other embodiments, the gene of interest is selected from the group consisting of a therapeutic gene (e.g., a gene that encodes a dystrophin polypeptide) or a reporter gene (e.g., GFP or tdTomato). In some embodiments, the gene of interest is a gene for delivery to a muscle cell.

In other embodiments, the pseudotyped exosome comprises a nucleic acid that can modulate (e.g., increase or decrease) gene expression (e.g., gRNA/Cas, gRNA/Cas9 or anti-sense oligonucleotides). In other embodiments, the pseudotyped particle comprises a nucleic acid that can decrease gene expression (e.g., gRNA/Cas, gRNA/Cas9 or anti-sense oligonucleotides). In certain embodiments, the nucleic acid that can modulate gene expression is gRNA/Cas (e.g., gRNA/Cas9), which is guide RNA (gRNA) and a CRISPR-associated endonuclease (Cas protein, such as Cas9). In still other embodiments, the gRNA can be a short synthetic RNA composed of a scaffold sequence for Cas-binding and, in certain embodiments, a user-defined 5-60 nucleotide spacer can define the genomic target to be modified. In some embodiments, one can change the genomic target of the Cas protein (e.g., Cas9 protein) by changing the target sequence present in the gRNA. In yet other embodiments, the anti-sense oligonucleotide can be synthetic DNA oligomers or synthetic RNA oligomers that hybridize to a target RNA in a sequence-specific manner. In certain embodiments, the anti-sense oligonucleotide can inhibit gene expression, modulate splicing of a precursor messenger RNA, and/or inactivate microRNAs. In certain embodiments, the length of an anti-sense oligonucleotide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, from 5 to 60, 10 to 50, 15 to 40, or 15 to 30 nucleotides long.

In some embodiments, the pseudotyped exosome exhibits fusogenic activity with a target cell upon binding of the myomaker polypeptide and/or the myomerger polypeptide to a myomaker polypeptide and/or a myomerger polypeptide on the target cell. In other embodiments, the target cell endogenously expresses a myomaker polypeptide and/or a myomerger polypeptide, optionally wherein the target cell is a muscle cell. In certain embodiments, the target cell does not endogenously express a myomaker polypeptide and/or a myomerger polypeptide, optionally wherein the target cell is a non-muscle cell. In other embodiments, the target cell can be but is not limited an animal cell, a vertebrate cell, a mammalian cell, a human cell, a rat cell, a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In yet other embodiments, the target cell can be but is not limited myoblasts (e.g., proliferative myoblasts), myotubes (e.g., differentiating myotube or differentiated myotubes), and fibroblasts. In still other embodiments, the target cell is a fibroblast, a mesenchymal stem cell (MSC), a hematopoietic stem cell, a blood cell, a bone marrow cell, or an adipose stem cell.

Pseudotyped Vesicular Stomatitis Virus (VSV)

Some embodiments of the invention include pseudotyped VSVs, as disclosed herein. In certain embodiments, the pseudotyped VSV surface comprises one or more myomerger polypeptides, one or more myomaker polypeptides, or a combination thereof. In certain embodiments, one or more myomaker polypeptides, one or more myomerger polypeptides, or a combination thereof is present on the surface of the pseudotyped VSV. In certain embodiments, one or more myomaker polypeptides, one or more myomerger polypeptides, or a combination thereof is present on the lipid envelope of the pseudotyped VSV. In other embodiments, the pseudotyped VSV does not have the native G-protein-coding gene. In still other embodiments, the pseudotyped VSV comprises the gene for green fluorescent protein.

In some embodiments of the pseudotyped VSV, the one or more myomerger polypeptides comprise a human myomerger polypeptide (e.g., SEQ ID NO:19). In other embodiments of the pseudotyped VSV, the one or more myomaker polypeptides comprise a human myomaker polypeptide (e.g., SEQ ID NO:1).

The pseudotyped VSV can be produced using any suitable method including but not limited to those disclosed herein. In other embodiments, the method for producing a pseudotyped VSV comprises contacting, in any order, (a) a composition comprising cells that express at least one myomerger polypeptide, at least one myomaker polypeptide, or a combination thereof, (b) a composition comprising one or more VSV production plasmids (e.g., one or more transfer plasmids or one or more packaging plasmids), (c) a composition comprising a nucleic acid encoding a gene of interest, and (d) optionally, a composition comprising one or more chemicals to increase transfection or transduction efficiency and then optionally recovering the pseudotyped VSV. In certain embodiments, the compositions of (b) and/or (c) are contacted with the composition of (d), and then the (b) and/or (c) with (d) mixture is contacted with composition of (a). In other embodiments, (i) the cells inducibly express at least one myomerger polypeptide, at least one myomaker polypeptide, or a combination thereof and (ii) an induction chemical is added to the composition of (a) prior to contacting (b) or (c) or (d), after (a) contacts (b), after (a) contacts (c), after (a) contacts (d), after (a) contacts (b) and/or (c) with (d), or a combination thereof. In some embodiments, the pseudotyped VSV can be produced by (a) contacting (e.g., any suitable manner of contacting including but not limited to adding, dropwise addition, mixing, injecting, spraying, blowing, or a combination thereof) a cell (e.g., BHK21 or HEK293t) that expresses at least one myomerger polypeptide, at least one myomaker polypeptide or a combination thereof with VSV (e.g., VSVAG or VSVAG-GFP/RFP) virus and (b) optionally recovering the pseudotyped VSV. In some embodiments, the cell (e.g., BHK21 or HEK293t) overexpresses at least one myomerger polypeptide and/or at least one myomaker polypeptide. In other embodiments, the contacting in step (a) occurs for at least about 1 hour, at least about 2 hours, at least about 8 hours, at least about 12 hours, at least about 24 hours, about 1 hour, about 2 hours, about 4 hours, about 8 hours, about 12 hours, about 24 hours, about 36 hours, about 48 hours, about 72 hours, or about 144 hours.

In yet other embodiments, the cell in step (a) is a cell that has been altered to express (e.g., fibroblast cells or 10T½ cells) one or more myomerger polypeptides, one or more myomaker polypeptides, or a combination thereof. In yet other embodiments, the cell in step (a) (e.g., BHK21) is a cell that has been altered to overexpress one or more myomerger polypeptides, one or more myomaker polypeptides, or a combination thereof. In other embodiments, the cell in step (a) can be but is not limited an animal cell, a vertebrate cell, a mammalian cell, a human cell, a rat cell, a mouse cell, a muscle cell, a non-muscle cell, a myoblast, a fibroblast, a BHK21 cell, an HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell.

In some embodiments, the method of producing a pseudotyped VSV comprising contacting a VSV virus (e.g., those disclosed herein, such as those comprising a gene of interest or a nucleic acid that can modulate gene expression) with a cell that expresses at least one myomerger polypeptide at least one myomaker polypeptide, or a combination thereof and optionally recovering the pseudotyped VSV, wherein the VSV virus optionally comprises a gene of interest or a nucleic acid that can modulate gene expression.

In some embodiments, the pseudotyped VSV can be recovered using any suitable method including but not limited to centrifugation, filtration, ultracentrifugation, ultrafiltration, chromatography, or hydrophobic interaction chromatography (HIC) (e.g., using a polyester, capillary-channeled polymer (PET C-CP) stationary phase), or a combination thereof.

In some embodiments, the method can optionally include the step of cryopreserving, before or after recovery. In other embodiments, the step of cryopreserving can include adding optional additives to the freezing media, such as but are not limited to sucrose, magnesium chloride, and combinations thereof.

In other embodiments, the pseudotyped VSV comprises a nucleic acid encoding a gene of interest or a nucleic acid that can modulate gene expression.

In other embodiments, the pseudotyped VSV comprises a nucleic acid encoding a gene of interest (e.g., a therapeutic gene, a reporter gene, a dystrophin nucleic acid molecule, a microdystrophin nucleic acid molecule, a minidystrophin nucleic acid molecule, a gene that is mutated in a genetic muscle disease (e.g., dystrophin, titin, or skeletal muscle alpha-actin), a gene for a secreted therapeutic factor, green fluorescent protein (GFP) or tdTomato). In yet other embodiments, the gene of interest does not encode for a myomaker protein or myomerger protein. In still other embodiments, the gene of interest is selected from the group consisting of a therapeutic gene (e.g., a gene that encodes a dystrophin polypeptide) or a reporter gene (e.g., GFP or tdTomato). In some embodiments, the gene of interest is a gene for delivery to a muscle cell.

In other embodiments, the pseudotyped VSV comprises a nucleic acid that can modulate (e.g., increase or decrease) gene expression (e.g., gRNA/Cas, gRNA/Cas9 or anti-sense oligonucleotides). In other embodiments, the pseudotyped particle comprises a nucleic acid that can decrease gene expression (e.g., gRNA/Cas, gRNA/Cas9 or anti-sense oligonucleotides). In certain embodiments, the nucleic acid that can modulate gene expression is gRNA/Cas (e.g., gRNA/Cas9), which is guide RNA (gRNA) and a CRISPR-associated endonuclease (Cas protein, such as Cas9). In still other embodiments, the gRNA can be a short synthetic RNA composed of a scaffold sequence for Cas-binding and, in certain embodiments, a user-defined 5-60 nucleotide spacer can define the genomic target to be modified. In some embodiments, one can change the genomic target of the Cas protein (e.g., Cas9 protein) by changing the target sequence present in the gRNA. In yet other embodiments, the anti-sense oligonucleotide can be synthetic DNA oligomers or synthetic RNA oligomers that hybridize to a target RNA in a sequence-specific manner. In certain embodiments, the anti-sense oligonucleotide can inhibit gene expression, modulate splicing of a precursor messenger RNA, and/or inactivate microRNAs. In certain embodiments, the length of an anti-sense oligonucleotide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, from 5 to 60, 10 to 50, 15 to 40, or 15 to 30 nucleotides long.

In some embodiments, the pseudotyped VSV exhibits fusogenic activity with a target cell upon binding of the myomaker polypeptide and/or the myomerger polypeptide to a myomaker polypeptide and/or a myomerger polypeptide on the target cell. In other embodiments, the target cell endogenously expresses a myomaker polypeptide and/or a myomerger polypeptide, optionally wherein the target cell is a muscle cell. In certain embodiments, the target cell does not endogenously express a myomaker polypeptide and/or a myomerger polypeptide, optionally wherein the target cell is a non-muscle cell. In other embodiments, the target cell can be but is not limited an animal cell, a vertebrate cell, a mammalian cell, a human cell, a rat cell, a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In yet other embodiments, the target cell can be but is not limited myoblasts (e.g., proliferative myoblasts), myotubes (e.g., differentiating myotube or differentiated myotubes), and fibroblasts. In still other embodiments, the target cell is a fibroblast, a mesenchymal stem cell (MSC), a hematopoietic stem cell, a blood cell, a bone marrow cell, or an adipose stem cell.

Pseudotyped Lentivirus

Some embodiments of the invention include pseudotyped lentivirus, as disclosed herein. In certain embodiments, the pseudotyped lentivirus surface comprises one or more myomerger polypeptides, one or more myomaker polypeptides, or a combination thereof. In certain embodiments, one or more myomaker polypeptides, one or more myomerger polypeptides, or a combination thereof is present on the surface of the pseudotyped lentivirus. In certain embodiments, one or more myomaker polypeptides, one or more myomerger polypeptides, or a combination thereof is present on the lipid envelope of the pseudotyped lentivirus.

In some embodiments of the pseudotyped lentivirus, the one or more myomerger polypeptides comprise a human myomerger polypeptide (e.g., SEQ ID NO:19). In other embodiments of the pseudotyped lentivirus, the one or more myomaker polypeptides comprise a human myomaker polypeptide (e.g., SEQ ID NO:1).

The pseudotyped lentivirus can be produced using any suitable method including but not limited to those disclosed herein. In some embodiments, the pseudotyped lentivirus can be produced by contacting (e.g., any suitable manner of contacting including but not limited to adding, dropwise addition, mixing, injecting, spraying, blowing, or a combination thereof), in any order, (a) a composition comprising cells (e.g., HEK293t or BHK21 cell) that express (e.g., or optionally inducibly expresses) at least one myomerger polypeptide, at least one myomaker polypeptide, or a combination thereof, (b) a composition comprising one or more lentivirus production plasmids (e.g., one or more transfer plasmids (e.g., plX304-GFP), one or more packaging plasmids (e.g., psPAX2), or a combination thereof), (c) a composition comprising a nucleic acid encoding a gene of interest (e.g., a dystrophin nucleic acid molecule, such as those disclosed herein) or a nucleic acid that can modulate gene expression (e.g., gRNA/Cas9 or anti-sense oligonucleotides) and (d) optionally, a composition comprising one or more chemicals to increase transfection or transduction efficiency (e.g., FuGENE 6 transfection reagent, Lipofectamine 2000, or Lipofectamine 3000). In other embodiments, (a), (b), (c) and (d) can be contacted in any suitable order. In certain embodiments, the compositions of (b) and/or (c) are contacted with the composition of (d), and then the (b) and/or (c) with (d) mixture is contacted with composition of (a). In yet other embodiments, where the cell inducibly expresses at least one myomerger polypeptide, at least one myomaker polypeptide, or a combination thereof, an induction chemical (e.g., dox) is added to the composition of (a) prior to contacting (b) or (c) or (d), after (a) contacts (b), after (a) contacts (c), after (a) contacts (d), after (a) contacts (b) and/or (c) with (d), or a combination thereof.

In other embodiments, in any of the contacting discussed herein (e.g., the contacting the compositions of (b) and/or (c) are contacted with the composition of (d), and then the (b) and/or (c) with (d) mixture is contacted with composition of (a)), the contacting time can occur for at least about 1 hour, at least about 2 hours, at least about 8 hours, at least about 12 hours, at least about 24 hours, about 1 hour, about 2 hours, about 4 hours, about 8 hours, about 12 hours, about 24 hours, about 36 hours, about 48 hours, about 72 hours, or about 144 hours.

In other embodiments, the cell in step (a) expresses or overexpresses one or more myomerger polypeptides, one or more myomaker polypeptides, or a combination thereof. In other embodiments, the cell in step (a) can be but is not limited myoblasts (e.g., proliferative myoblasts), myotubes (e.g., differentiating myotube or differentiated myotubes), and fibroblasts. In yet other embodiments, the cell in step (a) is a cell that has been altered to express (e.g., fibroblast cells or 10T½ cells) one or more myomerger polypeptides, one or more myomaker polypeptides, or a combination thereof. In yet other embodiments, the cell in step (a) is a cell that has been altered to overexpress one or more myomerger polypeptides, one or more myomaker polypeptides, or a combination thereof. In other embodiments, the cell in step (a) can be but is not limited an animal cell, a vertebrate cell, a mammalian cell, a human cell, a rat cell, a mouse cell, a muscle cell, a non-muscle cell, a myoblast, a fibroblast, an HEK293t cell, a BHK21 cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In some embodiments, the cell in step (a) is a modified cell (e.g., as disclosed herein).

In some embodiments, the method of producing a pseudotyped lentivirus comprising contacting a lentivirus virus (e.g., those disclosed herein, such as those comprising a gene of interest or a nucleic acid that can modulate gene expression) with a cell that expresses at least one myomerger polypeptide at least one myomaker polypeptide, or a combination thereof and optionally recovering the pseudotyped lentivirus, wherein the lentivirus virus optionally comprises a gene of interest or a nucleic acid that can modulate gene expression.

In some embodiments, the pseudotyped lentivirus can be recovered using any suitable method including but not limited to centrifugation, filtration, ultracentrifugation, ultrafiltration, chromatography, or hydrophobic interaction chromatography (HIC) (e.g., using a polyester, capillary-channeled polymer (PET C-CP) stationary phase), or a combination thereof.

In some embodiments, the method can optionally include the step of cryopreserving, before or after recovery. In other embodiments, the step of cryopreserving can include adding optional additives to the freezing media, such as but are not limited to sucrose, magnesium chloride, and combinations thereof.

In other embodiments, the pseudotyped lentivirus comprises a nucleic acid encoding a gene of interest or a nucleic acid that can modulate gene expression.

In other embodiments, the pseudotyped lentivirus comprises a nucleic acid encoding a gene of interest (e.g., a therapeutic gene, a reporter gene, a dystrophin nucleic acid molecule, a microdystrophin nucleic acid molecule, a minidystrophin nucleic acid molecule, a gene that is mutated in a genetic muscle disease (e.g., dystrophin, titin, or skeletal muscle alpha-actin), a gene for a secreted therapeutic factor, green fluorescent protein (GFP) or tdTomato). In yet other embodiments, the gene of interest does not encode for a myomaker protein or myomerger protein. In still other embodiments, the gene of interest is selected from the group consisting of a therapeutic gene (e.g., a gene that encodes a dystrophin polypeptide) or a reporter gene (e.g., GFP or tdTomato). In some embodiments, the gene of interest is a gene for delivery to a muscle cell.

In other embodiments, the pseudotyped lentivirus comprises a nucleic acid that can modulate (e.g., increase or decrease) gene expression (e.g., gRNA/Cas, gRNA/Cas9 or anti-sense oligonucleotides). In other embodiments, the pseudotyped particle comprises a nucleic acid that can decrease gene expression (e.g., gRNA/Cas, gRNA/Cas9 or anti-sense oligonucleotides). In certain embodiments, the nucleic acid that can modulate gene expression is gRNA/Cas (e.g., gRNA/Cas9), which is guide RNA (gRNA) and a CRISPR-associated endonuclease (Cas protein, such as Cas9). In still other embodiments, the gRNA can be a short synthetic RNA composed of a scaffold sequence for Cas-binding and, in certain embodiments, a user-defined 5-60 nucleotide spacer can define the genomic target to be modified. In some embodiments, one can change the genomic target of the Cas protein (e.g., Cas9 protein) by changing the target sequence present in the gRNA. In yet other embodiments, the anti-sense oligonucleotide can be synthetic DNA oligomers or synthetic RNA oligomers that hybridize to a target RNA in a sequence-specific manner. In certain embodiments, the anti-sense oligonucleotide can inhibit gene expression, modulate splicing of a precursor messenger RNA, and/or inactivate microRNAs. In certain embodiments, the length of an anti-sense oligonucleotide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, from 5 to 60, 10 to 50, 15 to 40, or 15 to 30 nucleotides long.

In some embodiments, the pseudotyped lentivirus exhibits fusogenic activity with a target cell upon binding of the myomaker polypeptide and/or the myomerger polypeptide to a myomaker polypeptide and/or a myomerger polypeptide on the target cell. In other embodiments, the target cell endogenously expresses a myomaker polypeptide and/or a myomerger polypeptide, optionally wherein the target cell is a muscle cell. In certain embodiments, the target cell does not endogenously express a myomaker polypeptide and/or a myomerger polypeptide, optionally wherein the target cell is a non-muscle cell. in other embodiments, the target cell can be but is not limited an animal cell, a vertebrate cell, a mammalian cell, a human cell, a rat cell, a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell. In still other embodiments, the target cell is a fibroblast, a mesenchymal stem cell (MSC), a hematopoietic stem cell, a blood cell, a bone marrow cell, or an adipose stem cell.

Dystrophin Polypeptides and Dystrophin Nucleic Acid Molecules

Some embodiments of the invention include pseudotyped particles (e.g., pseudotyped exosomes, pseudotyped VSV, or pseudotyped lentivirus) comprising the dystrophin polypeptide, the dystrophin nucleic acid molecule, or both, cells comprising the dystrophin polypeptide, the dystrophin nucleic acid molecule, or both, or using the dystrophin polypeptide, the dystrophin nucleic acid molecule, or both. In some embodiments, the dystrophin polypeptide is a microdystrophin polypeptide or a minidystrophin polypeptide. The term “dystrophin polypeptide” encompasses “wt-dystrophin polypeptides” (i.e., dystrophin polypeptides found in nature without any purposely human-made modification) and “mutant dystrophin polypeptides” (e.g., with one or more modifications made to a wt-dystrophin polypeptide, such as any of the modifications disclosed above for the myomerger polypeptide and/or myomaker polypeptide). In other embodiments, the dystrophin polypeptide has at least one amino acid modification relative to a wt-dystrophin polypeptide (e.g., any of those disclosed above for the myomerger polypeptide and/or myomaker polypeptide, such as conservative substitutions). A wt-dystrophin polypeptide can, in some embodiments, be a dystrophin polypeptide from any animal including but not limited to a mammal, a rat, a cat, a rabbit, a human, a cow, a chicken, a turkey, a monkey, a tree shrew, a dog, a pig, a shrew, an elephant, or an opossum. In certain embodiments, the dystrophin polypeptide is in a pseudotyped particle, such as an exosome, a VSV, or a lentivirus.

Nucleic acid molecules that encode for the dystrophin polypeptide are termed “dystrophin nucleic acid molecules.” In certain embodiments, the dystrophin nucleic acid molecule is included in a vector (e.g., a viral vector, a retroviral vector, a lentiviral vector, a VSV vector, an adenoviral vector, an adeno-associated viral vector, a herpesviral vector, a chimeric viral vector, a plasmid, an expression vector, a conjugative vector, or a nonconjugative vector). In certain embodiments, the dystrophin nucleic acid molecule is in a pseudotyped particle, such as an exosome, a VSV, or a lentivirus (e.g., as a gene of interest). In certain embodiments, the dystrophin nucleic acid molecule is in a cell, such as an insect cell (e.g., an Sf9 cell) or mammalian cell (e.g., a human cell, a rat cell a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, or an adipose stem cell).

Cells Including Modified Cells

Some embodiments of the invention include cells such as modified cells. In certain embodiments, a modified cell is a cell that comprises one or more modifications of a cell, where at least one of the one or more modifications was implemented by a human (e.g., by human activity, either directly or indirectly). In some embodiments, the cell to be modified can be an unmodified cell or can be a cell that has been previously modified (e.g., modified as disclosed herein). A cell can be modified in any desired manner, including but not limited to (a) adding a nucleic acid molecule such as but not limited to one or more nucleic acid molecules disclosed herein (a myomaker nucleic acid molecule, a myomerger nucleic acid molecule, one or more nucleic acids encoding proteins for pseudotyped particle production, a gene of interest (e.g., dystrophin nucleic acid molecule), a nucleic acid that can modulate gene expression (e.g., gRNA/Cas9 or anti-sense oligonucleotides), or a combination thereof), (b) adding one or more polypeptides, including but not limited to polypeptides disclosed herein (e.g., a myomaker polypeptide, a myomerger polypeptide, a dystrophin polypeptide or a combination thereof), (c) expressing (e.g., overexpressing) one or more polypeptides (e.g., a myomaker polypeptide, a myomerger polypeptide, a dystrophin polypeptide or a combination thereof), or (d) a combination thereof (e.g., overexpressing a myomaker polypeptide and overexpressing a myomerger polypeptide). In some instances, a modified cell can result from a further modification of another modified cell.

Adding a nucleic acid molecule to modify a cell can be accomplished using any suitable method including but not limited to one or more of transformation (as used herein transfection methods are encompassed by the term transformation), viral transformation (e.g., using a viral vector, a retroviral vector, a lentiviral vector, a VSV vector, an adenoviral vector, an adeno-associated viral vector, a herpesviral vector, a chimeric viral vector, a plasmid, a cosmid, an artificial chromosome, a bacteriophage, a virus, an animal virus, a plant virus, an expression vector, a conjugative vector, or a nonconjugative vector), injection, microinjection, electroporation, sonication, calcium ion treatment, calcium phosphate precipitation, PEG-DMSO treatment, DE-Dextran treatment, liposome mediated transformation, or a receptor mediated transformation. Adding a polypeptide to modify a cell can be accomplished using any suitable method including but not limited to one or more of injection, microinjection, electroporation, sonication, calcium ion treatment, calcium phosphate precipitation, PEG-DMSO treatment, DE-Dextran treatment, or liposome mediated. The added nucleic acid molecule can be part of a vector (e.g., a viral vector, a retroviral vector, a lentiviral vector, a VSV vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, or a nonconjugative vector), a plasmid, a cosmid, an artificial chromosome, a bacteriophage, a virus, an animal virus, or a plant virus. In some embodiments, the added nucleic acid molecule is exogenous; “exogenous” means (a) that the added nucleic acid molecule originates from outside of the cell (e.g., is foreign to the cell) or (b) that the added nucleic acid molecule can be found inside the cell, but the added nucleic acid molecule is placed in the cell where it is not normally found (e.g., a different part of the chromosome or on an added plasmid). In some embodiments, the added polypeptide is exogenous; “exogenous” in this context means that the added polypeptide originates from outside of the cell (e.g., is foreign to the cell).

In some embodiments, the modified cell comprises one or more nucleic acids encoding proteins for pseudotyped particle production and nucleic acid encoding a myomaker polypeptide and/or a myomerger polypeptide. In other embodiments, the encoded myomaker polypeptide and/or myomerger polypeptide is transiently expressed by the modified cell. In yet other embodiments, the encoded myomaker polypeptide and/or myomerger polypeptide is expressed from a plasmid. In still other embodiments, the encoded myomaker polypeptide and/or myomerger polypeptide is stably expressed by the modified cell. In certain embodiments, the encoded myomaker polypeptide and/or myomerger polypeptide is expressed from an endogenous locus. In certain embodiments, the myomaker polypeptide and/or myomerger polypeptide is overexpressed. In other embodiments, the encoded myomaker polypeptide and/or myomerger polypeptide is inducibly expressed by the modified cell. In certain embodiments, the nucleic acid encoding a myomaker polypeptide and/or myomerger polypeptide is linked to an inducible response element, optionally a promoter. In other embodiments, the nucleic acid encoding a myomaker polypeptide and/or myomerger polypeptide is linked to an inducible response element (e.g., a doxycycline response element or promoter).

Some embodiments of the invention include a modified cell comprising and a nucleic acid encoding a myomaker and/or myomerger polypeptide, wherein the nucleic acid encoding a myomaker and/or myomerger polypeptide is linked to an inducible response element, optionally a promoter. In other embodiments, the inducible response element is a doxycycline response element (e.g., a doxycycline response element or promoter).

The cell to be modified can be any suitable cell including but not limited to an insect cell (e.g., an Sf9 cell), a vertebrate cell, or a mammalian cell (e.g., a human cell, a rat cell a mouse cell, a muscle cell, a non-muscle cell, a myoblast, a fibroblast, a BHK21 cell, a HEK293t cell, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell). In certain embodiments, an unmodified cell can be any suitable cell including but not limited insect cell, a vertebrate cell, or a mammalian cell (e.g., a human cell, a rat cell a mouse cell, a muscle cell, a non-muscle cell, a myoblast, a fibroblast, a BHK21 cell, a HEK293t cell, a NIH/3T3 cell, a CHO cell, a mesenchymal stem cell (MSC), a hematopoietic stem cell, a blood cell, a bone marrow cell, a stem cell, or an adipose stem cell).

In some embodiments, a modified cell can be but is not limited to a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a BHK21 cell, a modified BHK21 cell, a HEK293t cell, a modified HEK293t cell, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In other embodiments, the modified cell is a modified non-muscle cell (e.g., a modified fibroblast, a BHK21 cell, a modified BHK21 cell, a HEK293t cell, a modified HEK293t cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell).

In other embodiments, the modified cell is (a) a non-muscle cell with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added, (b) a stem cell with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added, (c) a fibroblast with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added, (d) a muscle cell with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added, (e) a myoblast cell with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added, or (f) a MSC cell with one or more molecules of a myomaker nucleic acid molecule, a myomerger nucleic acid molecule or one or more nucleic acids encoding proteins for pseudotyped particle production (e.g., where one or more of the myomaker nucleic acid molecule, myomerger nucleic acid molecule and/or one or more nucleic acids encoding proteins for pseudotyped particle production is exogenous and/or one or more nucleic acids comprises a gene of interest or a nucleic acid that can modulate gene expression) are added.

The modified cell can be prepared using any suitable method including but not limited to those disclosed herein or those found in LI et al. 2005, which is herein incorporated by reference in its entirety (LI et al. (2005) “Stable transduction of myogenic cells with lentiviral vectors expressing a minidystrophin” Gene Therapy, Vol. 12, pp. 1099-1108.) (e.g., using the lentiviral vector with a human CMV promotor or a murine stem cell virus promoter (MSCV)) to modify or partially modify a cell.

Compositions Including Pharmaceutical Compositions

One or more pseudotyped exosomes, one or more pseudotyped VSV, one or more pseudotyped lentivirus, one or more modified cells, or a combination thereof can be part of a composition and can be in an amount (by weight of the total composition) individually or as a whole, of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

One or more pseudotyped exosomes, one or more pseudotyped VSV, one or more pseudotyped lentivirus, one or more modified cells, or a combination thereof can be purified or isolated in an amount (by weight of the total composition) individually or as a whole, of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%. In some embodiments, isolated or purified means that impurities (e.g., cell components or unwanted solution components if chemically synthesized) were removed or partially removed by one or more of any suitable technique (e.g., column chromatography, HPLC, centrifugation, fractionation, gel, precipitation, or salting out).

Some embodiments of the present invention include compositions comprising one or more pseudotyped exosomes, one or more pseudotyped VSV, one or more pseudotyped lentivirus, one or more modified cells, or a combination thereof. In certain embodiments, the composition is a pharmaceutical composition, such as compositions that are suitable for administration to animals (e.g., mammals, primates, monkeys, humans, canine, porcine, mice, rabbits, or rats). In some embodiments, there may be inherent side effects (e.g., it may harm the patient or may be toxic or harmful to some degree in some patients).

In some embodiments, one or more pseudotyped exosomes, one or more pseudotyped VSV, one or more pseudotyped lentivirus, one or more modified cells, or a combination thereof can be part of a pharmaceutical composition and can be in an amount (by weight of the total composition) individually or as a whole, of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%.

In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients (e.g., pharmaceutically acceptable carriers or pharmaceutically acceptable excipients). A “formulary ingredient” can be any suitable ingredient (e.g., suitable for the drug(s), for the dosage of the drug(s), for the timing of release of the drugs(s), for the disease, for the disease state, for the organ, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. In some embodiments, the concentration of any individual formulary ingredient in a composition (e.g., pharmaceutical composition) can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the concentration of at least one formulary ingredient is not that same as that found in a natural system. In some embodiments, the concentration of at least one formulary ingredient is not that same as that found in one or more natural systems (e.g., any natural system found in nature) is found.

In certain embodiments, pharmaceutical compositions can be formulated to release the active ingredient (e.g., one or more pseudotyped exosomes, one or more pseudotyped VSV, one or more pseudotyped lentivirus, one or more modified cells, or a combination thereof) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Methods of Using Pseudotyped Particles

Some embodiments of the invention include methods of using pseudotyped particles, such as pseudotyped exosomes, pseudotyped VSV or pseudotyped lentivirus. Some embodiments of the invention include methods that comprise contacting the one or more pseudotyped particles (e.g., as disclosed herein) with a target cell. Some embodiments of the invention include methods for administering one or more pseudotyped particles (e.g., pseudotyped exosomes, pseudotyped VSV or pseudotyped lentivirus, as disclosed herein) to an animal.

Certain embodiments of the invention include methods for mediating fusion of a pseudotyped particle with a cell, the method comprising contacting the pseudotyped particle (e.g., as disclosed herein) with a target cell. Other embodiments of the invention include methods of delivering a gene of interest or a nucleic acid that can modulate gene expression to a cell, the method comprising contacting the pseudotyped particle (e.g., as disclosed herein) with a target cell. In some embodiments, contacting occurs in vitro or in vivo. In other embodiments, the target cell endogenously expresses a myomaker polypeptide and/or a myomerger polypeptide and optionally the target cell is a muscle cell. In still other embodiments, the target cell is a muscle cell, a myoblast, a myotube, or a mesenchymal stem cell (MSC). In yet other embodiments, the target cell does not endogenously express a myomaker polypeptide and/or a myomerger polypeptide and optionally the target cell is a non-muscle cell. In certain embodiments, the target cell is a non-muscle cell, a fibroblast, a mesenchymal stem cell (MSC), a hematopoietic stem cell, a blood cell, a bone marrow cell, or an adipose stem cell. In some embodiments, the target cell can be an insect cell (e.g., an Sf9 cell), a vertebrate cell, or a mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a dendritic cell, a cancer cell, a mesenchymal stem cell (MSC), a hematopoietic stem cell, a blood cell, a bone marrow cell, a stem cell, or an adipose stem cell). In some embodiments, the target cell under expresses dystrophin (e.g., microdystrophin or minidystrophin) does not express dystrophin (e.g., microdystrophin or minidystrophin), or expresses a defective form of dystrophin (e.g., microdystrophin or minidystrophin).

In some embodiments of administering, of contacting, or of mediating fusion, the pseudotyped particle comprises a nucleic acid molecule comprising a gene of interest (e.g., dystrophin nucleic acid molecule, any gene mutated in genetic muscle diseases, a gene for a secreted therapeutic factor) or a nucleic acid that can modulate gene expression (e.g., gRNA/Cas9 or anti-sense oligonucleotides). In other embodiments, the gene of interest encodes a dystrophin polypeptide. In certain embodiments, the gene of interest encodes a microdystrophin or a minidystrophin. In still other embodiments, the pseudotyped particle is a pseudotyped exosome. In certain embodiments, the pseudotyped particle is a pseudotyped exosome and the pseudotyped exosome comprises a polypeptide of interest (e.g., a dystrophin polypeptide) and/or a gene of interest (e.g., a dystrophin nucleic acid molecule, any gene mutated in genetic muscle diseases, a gene for a secreted therapeutic factor) and/or a nucleic acid that can modulate gene expression (e.g., gRNA/Cas9 or anti-sense oligonucleotides). In still other embodiments, the pseudotyped particle is a pseudotyped VSV. In some embodiments, the pseudotyped particle is a pseudotyped VSV and the pseudotyped VSV comprises a nucleic acid molecule comprising a gene of interest (e.g., dystrophin nucleic acid molecule, any gene mutated in genetic muscle diseases, a gene for a secreted therapeutic factor) and/or a nucleic acid that can modulate gene expression (e.g., gRNA/Cas9 or anti-sense oligonucleotides). In certain embodiments, the pseudotyped particle is a pseudotyped VSV and the pseudotyped VSV comprises a nucleic acid molecule comprising a dystrophin nucleic acid molecule. In still other embodiments, the pseudotyped particle is a pseudotyped lentivirus. In some embodiments, the pseudotyped particle is a pseudotyped lentivirus and the pseudotyped lentivirus comprises a nucleic acid molecule comprising a gene of interest (e.g., dystrophin nucleic acid molecule, any gene mutated in genetic muscle diseases, a gene for a secreted therapeutic factor) and/or a nucleic acid that can modulate gene expression (e.g., gRNA/Cas9 or anti-sense oligonucleotides). In certain embodiments, the pseudotyped particle is a pseudotyped lentivirus and the pseudotyped lentivirus comprises a nucleic acid molecule comprising a dystrophin nucleic acid molecule.

The administering of the one or more pseudotyped particles (e.g., pseudotyped exosomes, pseudotyped VSV or pseudotyped lentivirus, as disclosed herein) in the method can occur by any suitable manner, such as but not limited to those disclosed herein. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intranasal administration, or intramuscular administration (e.g., intramuscular injection). In certain embodiments, the delivery comprises an injection or an intramuscular injection. In certain embodiments, the delivery comprises an injection comprising the one or more pseudotyped particles (e.g., pseudotyped exosomes, pseudotyped VSV or pseudotyped lentivirus, as disclosed herein) (e.g., in a composition or in a pharmaceutical composition). In other embodiments, the delivery comprises an intramuscular injection comprising the one or more pseudotyped particles (e.g., pseudotyped exosomes, pseudotyped VSV or pseudotyped lentivirus, as disclosed herein) (e.g., in a composition or in a pharmaceutical composition). For example, the administering can be accomplished by implanting, injecting, or grafting the one or more modified cells in an animal. Any suitable administration route can be used, including but not limited to those disclosed herein.

Animals include but are not limited to mammals, primates, monkeys (e.g., macaque, rhesus macaque, or pig tail macaque), humans, canine, feline, bovine, porcine, avian (e.g., chicken), mice, rabbits, and rats. In some embodiments, the animal is a mouse, rat, or human. As used herein, the term “subject” refers to both human and animal subjects.

In certain embodiments, the method to administer can be part of a treatment of a disease. In some embodiments, the disease can be any disease, such as but not limited to, diseases where cells under express dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In some embodiments, the disease can be a non-muscle-related disease, such as but not limited to, non-muscle-related diseases where cells under express dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In some embodiments, the disease can be a muscle-related disease, such as but not limited to, muscle-related diseases where cells under express dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In certain embodiments, the treated disease can be a myopathy, muscular dystrophy, amyotrophic lateral sclerosis (ALS or also called Lou Gehrig's disease), glycogen storage disease type II (also called Pompe disease), rhabdomyosarcoma (RMS), sarcopenia, or a combination thereof. In some embodiments, the disease can be cancer. As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a disease, decrease in severity of a disease, or preventing, preventively ameliorating symptoms, or otherwise reducing the risk of developing a particular disease. As used herein, reference to “treating” an animal includes but is not limited to prophylactic treatment and therapeutic treatment. In some embodiments, “treating” does not include preventing disease and/or prophylactic treatment. Any of the methods or compositions (e.g., pharmaceutical compositions) described herein can be used to treat an animal.

Animals that can be treated include but are not limited to mammals, primates, monkeys (e.g., macaque, rhesus macaque, or pig tail macaque), humans, canine, feline, bovine, porcine, avian (e.g., chicken), mice, rabbits, and rats. In some embodiments, the animal is a mouse, rat, or human.

In yet other embodiments, the administering of one or more pseudotyped particles (e.g., pseudotyped exosomes, pseudotyped VSV or pseudotyped lentivirus, as disclosed herein) can occur by any suitable administration route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intranasal administration, or intramuscular administration (e.g., intramuscular injection). In certain embodiments, the delivery comprises an injection or an intramuscular injection. In certain embodiments, the delivery comprises an injection comprising the modified cell (e.g., in a composition or in a pharmaceutical composition). In other embodiments, the delivery comprises an intramuscular injection comprising the modified cell (e.g., in a composition or in a pharmaceutical composition).

In still other embodiments, the treating can further comprise one or more of the administering steps.

The presently-disclosed subject matter is further illustrated by the following specific but non-limiting examples. The following examples may include compilations of data that are representative of data gathered at various times during the course of development and experimentation related to the present invention.

### EXAMPLES

Materials and Methods

Myomaker and Myomerger Sequences

Unless otherwise indicated the nucleic acid sequence used in the examples for myomaker is SEQ ID NO:10. Unless otherwise indicated the nucleic acid sequence used in the examples for myomerger is SEQ ID NO:25.

Generation of Myomaker+Myomerger+(Exosome Producing) Cell Lines

5×10⁶ PE cells were plated in a 10 cm dish with 10% FBS/DMEM and transfected with pBabeX-Empty, pBabeX-Myomaker, or pBabeX-Myomerger using FuGENE-6 (ratio of 1:3 ugDNA to ul FuGENE-6). After 48 hours, viral supernatant was collected and centrifuged at 2500 RPM for 5 minutes at RT to clear cellular debris. The viral supernatant was then passed through a 0.45 um SFCA filter. Virus was then applied to C2C12 myoblasts or 10T ½ fibroblasts to generate cell lines with increased expressed of Myomaker and Myomerger. Expression of Myomaker and Myomerger in cell lines was determined by western blot.

Isolation of Myomaker+/Myomerger+Exosomes from C2C12 Myotubes and 10T½ Fibroblasts

For exosome isolation, C2C12 myotubes or 10T ½ fibroblasts were incubated with media containing exosome depleted serum for 48 hours. Collect culture supernatant containing exosomes produced by each cell line was collected into 50 ml conical tubes. Tubes were centrifuged at 300 g, 4° C. for 10 min to pellet cellular debris. Supernatants were then transferred into new tubes and centrifuged at 1000 g, 4° C. for 30 min to pellet undesired micro contaminants. Supernatants were then filtered through 0.22 um filters and loaded into tubes for ultra-centrifugation (100K g, 4° C., overnight). Pellets were washed and resuspended in NP-40 buffer for analysis of Myomaker and Myomerger expression through standard western blot procedures or pellets (exosomes) were labeled with DiI (a lipid dye). The DiI-labeled exosomes were placed on C2C12 myoblasts, C2C12 myotubes, or 10T ½ fibroblasts to assess delivery of material to those cell types.

Generation of Myomaker+Myomerger+Pseudotyped Virus

For each pseudotype, 2.5×10⁶ inducible HEK293T cells/dish (60 mm) were plated. Inducible HEK293T cell lines include iEmpty, iMymk, iMymg, and iMymk+iMymg that were generated by transducing HEK cells with a virus containing pLVX-Empty, pLVX-Mymk, pLVX-Mymg, or pLVX-Mymk+Mymg. To induce expression of Mymk or Mymg, cells were treated with 1 μg/ml Dox for 2 hours before transfection of lentivirus transfer (pLX304-GFP) and packaging plasmids (psPAX2). Viral supernatants were collected 24 hours, 48 hours, and 72 hours after transfection, and combined for downstream analysis. Viral supernatants were centrifuged at 2500 RPM for 2.5 minutes at RT to remove cellular debris, and then filtered with a 0.45 uM SFCA syringe filter. Viral transduction was measured by placing virus on iMynk+Mymg target cells and GFP+ cells were counted. For in vivo experiments, virus was concentrated through centrifugation at 10,000 RPM for at least 4 hours at 4° C., then washed, and resuspended in 1×PBS for either intramuscular delivery.

For VSVAG virus, BHK21 cells expressing Empty, Mymk, Mymg, or Mymk+Mymg were transduced with VSVAG-GFP/RFP virus for 24 hours. Newly generated VSVAG virus (pseudotyped with Mymk and/or Mymg) was collected, centrifuged as above, and incubated on target cells as described above.

Assessment of Myomaker+Myomerger+Pseudotyped Lentivirus Transduction In Vitro and In Vivo

Viruses encoding for GFP were incubated with myoblasts, myotubes, or fibroblasts to assess transduction in vitro. GFP+ cells were counted to estimate viral titer. To assess transduction in vivo, viruses encoding for Cre recombinase were generated and intramuscularly injected into the tibialis anterior of mdx; Rosa^{TdTom} mice or Rosa^{TdTom} mice. Viruses were injected with and without prior injury by cardiotoxin. The Rosa^{TdTom} mouse strain is a Cre-dependent reporter where transduced cells will be Tomato+. Transduction was monitored by evaluating the number of transduced cells. Viruses encoding for luciferase were also generated and monitored in real-time through bioluminescence imaging.

### Example 1—Exosomes

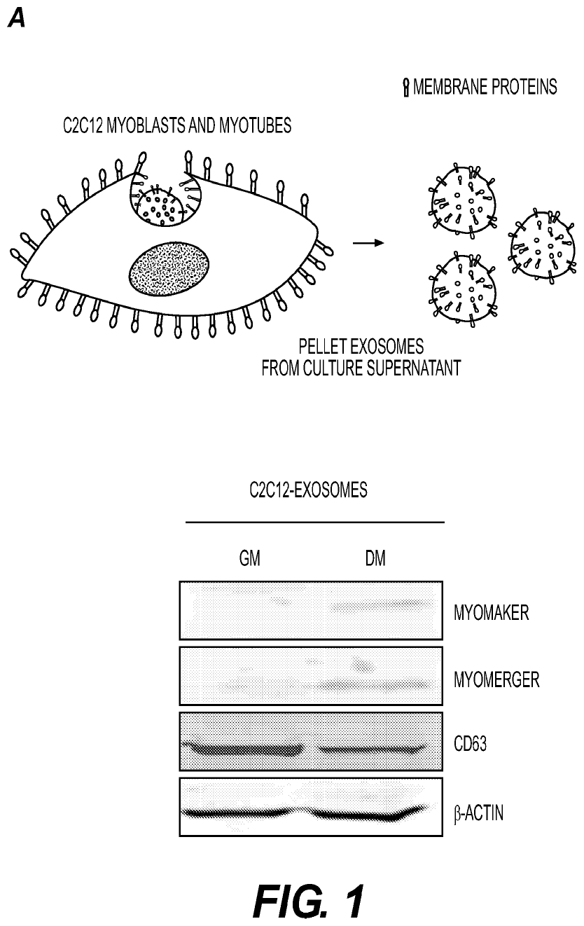

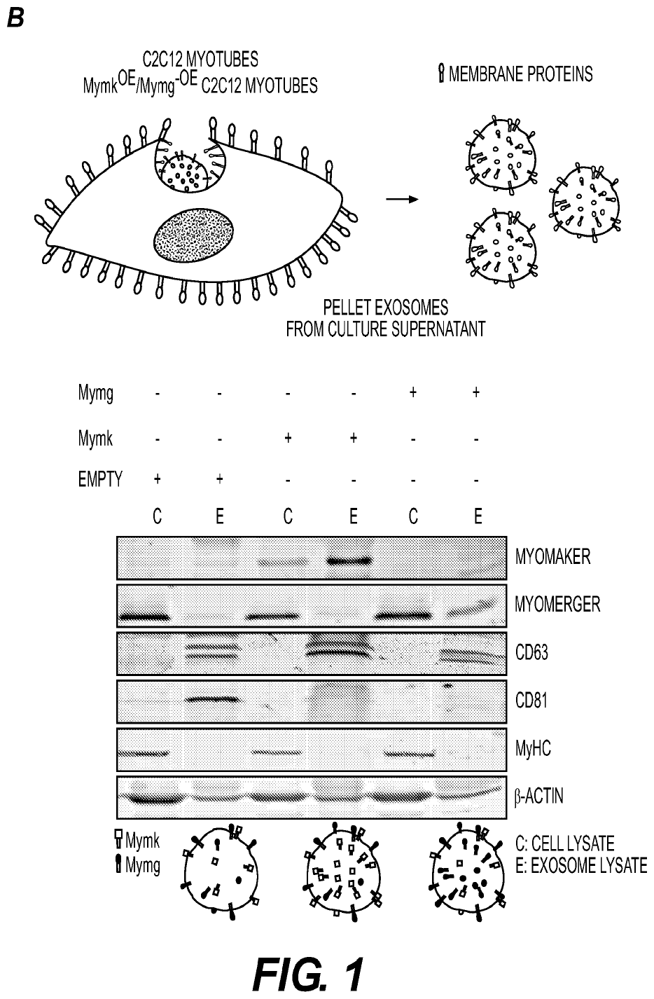

To investigate whether Myomaker and/or Myomerger naturally track to exosomal membranes and whether or not they may contribute to enhancing the transduction capacity of myogenic-cell derived exosomes to myogenic targets, we collected exosomes from culturing media of proliferating C2C12 myoblasts and differentiated myotubes and assayed for Myomaker and Myomerger via immunoblot. Our findings revealed that both Myomaker and Myomerger localize to exosomes from differentiating C2C12 myotubes and to a lesser extent in exosomes collected from those of proliferating cells consistent with the cellular expression of these proteins during myogenesis (FIG. 1A). As a result, we chose to modify Myomaker and/or Myomerger levels on exosomal membranes through direct overexpression of the proteins in exosome-producing cells as confirmed in (FIG. 1B).

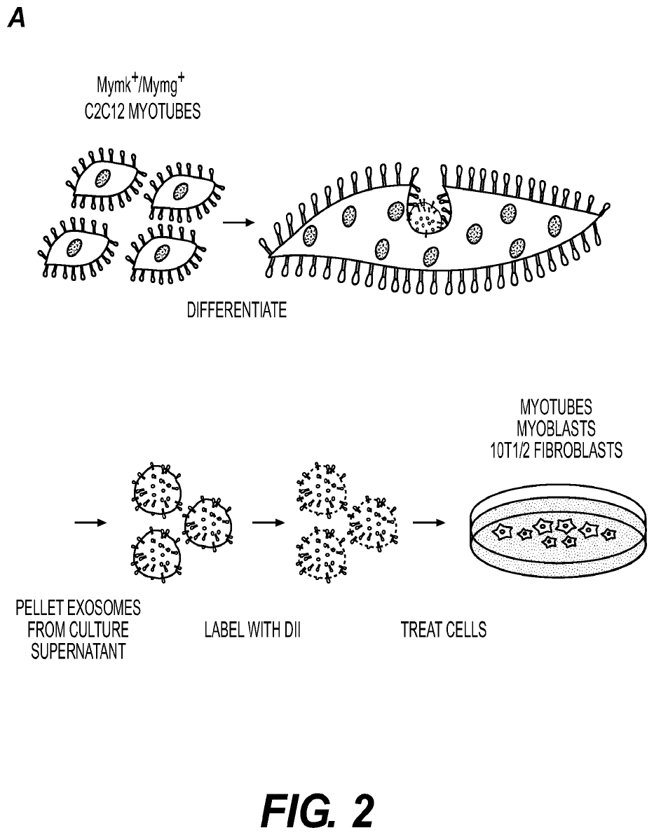

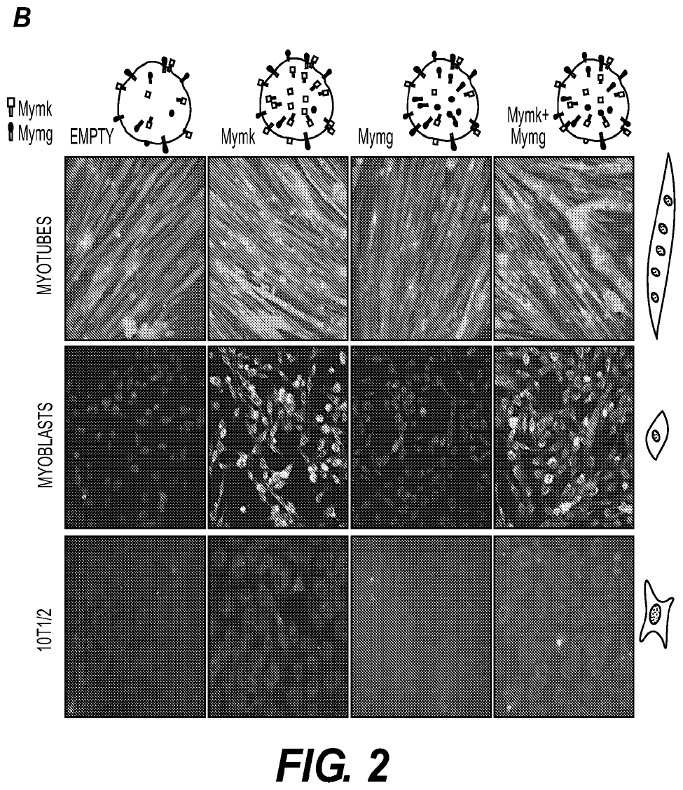

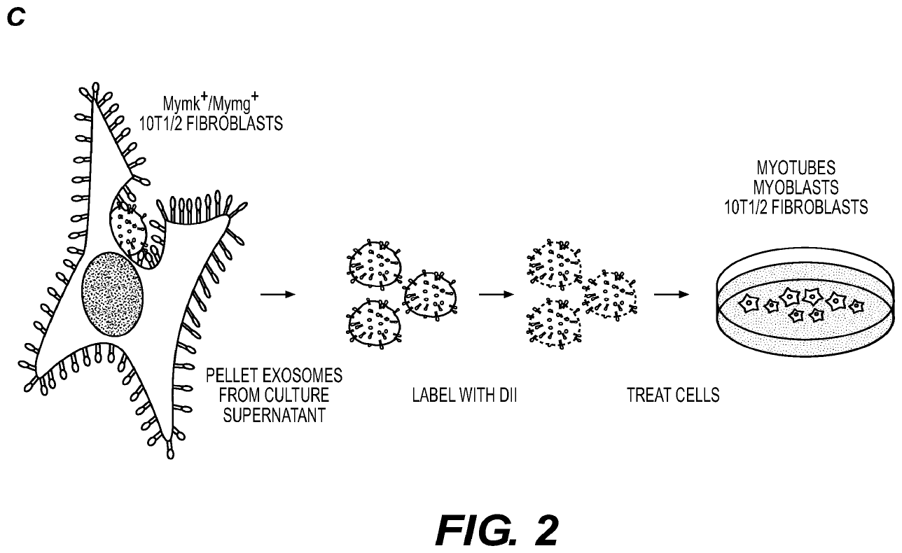

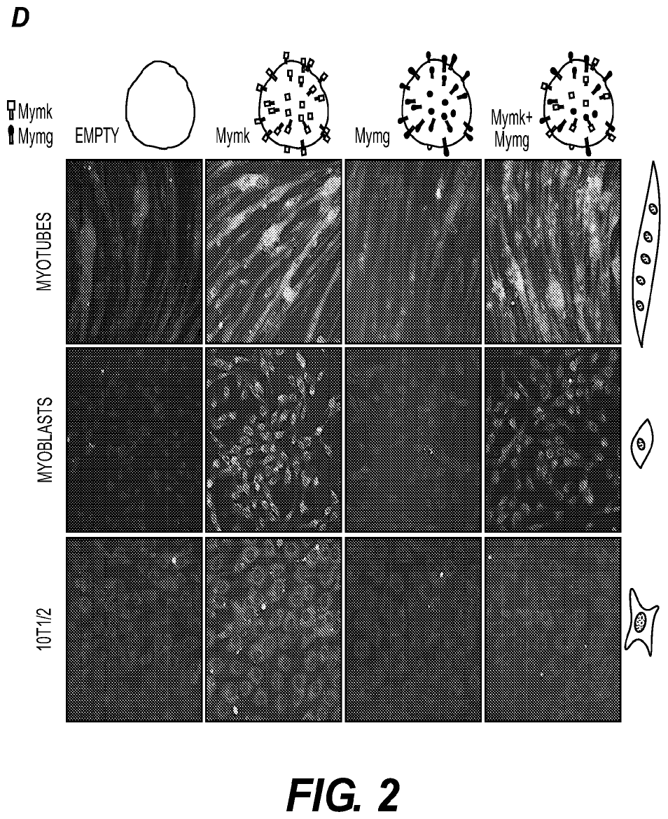

We next investigated whether Myomaker and Myomerger influence exosome internalization by myogenic cells. We purified exosomes from WT C2C12s or those overexpressing Myomaker and/or Myomerger and labeled them with DiI, a lipophilic membrane stain, to visualize exosomal up-take by various cells (FIG. 2A). Exosomes purified from WT C2C12 myotubes displayed higher internalization in proliferating and fusing myoblast and minimal to no uptake by fibroblasts. Forced expression of Myomaker or Myomaker with Myomerger in exosomes further enhanced exosomal uptake by myogenic cells only (FIG. 2B). To determine if these results are primarily owed to Myomaker and Myomerger, we expressed these proteins together or separately in 10T½ fibroblasts and collected and labeled exosomes as described above (FIG. 2C). Consistent with our previous findings, forced expression of Myomaker or Myomaker+Myomerger increased exosomal uptake only in myogenic cells while exosomes deficient of Myomaker+Myomerger exhibited poor uptake by myogenic targets (FIG. 2D). Together, these findings indicate that exosomes coated with Myomaker or Myomaker with Myomerger are functional and enhance exosomal uptake by myogenic cells.

### Example 2—VSVAG

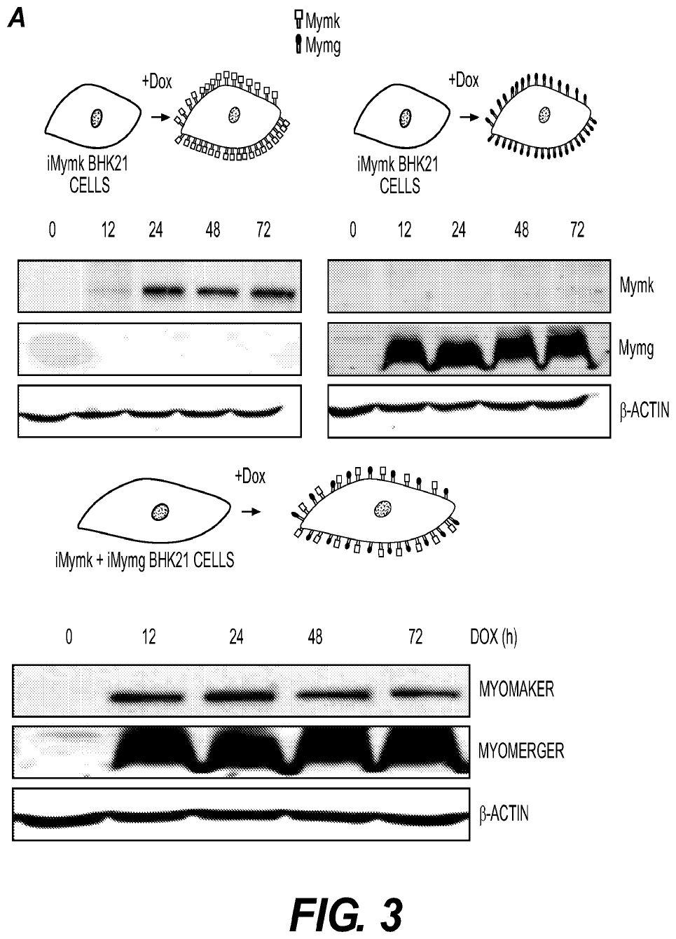

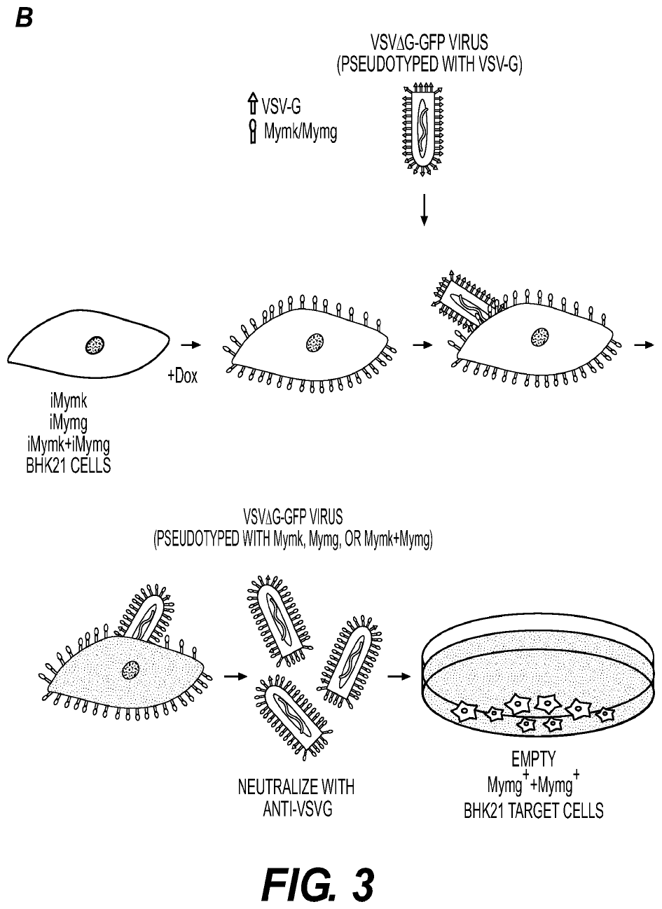

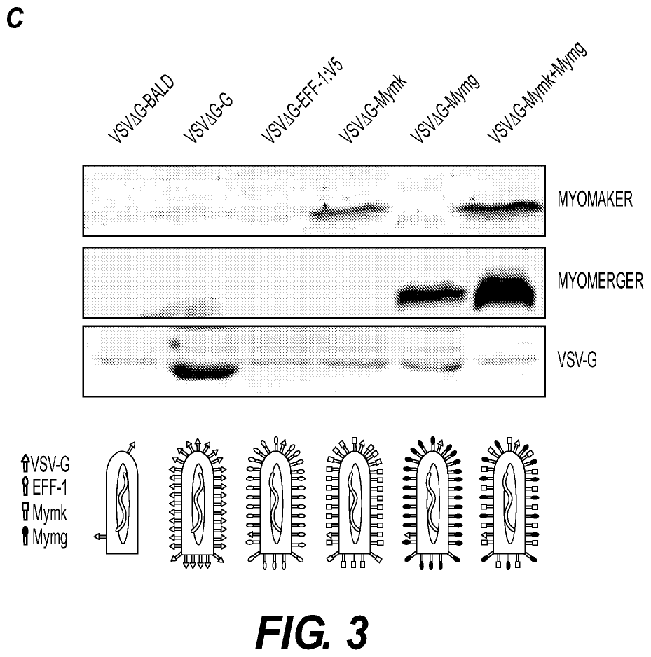

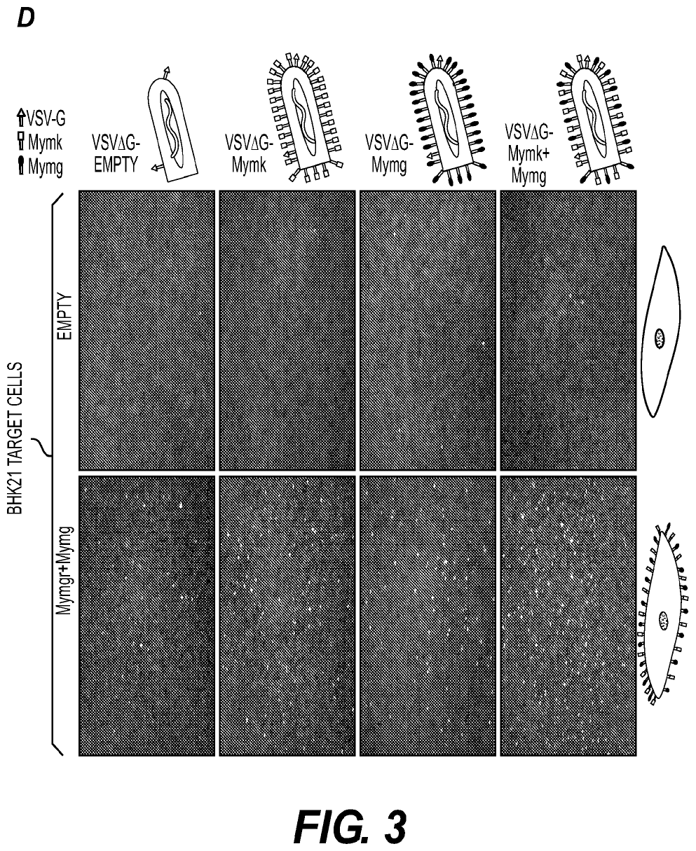

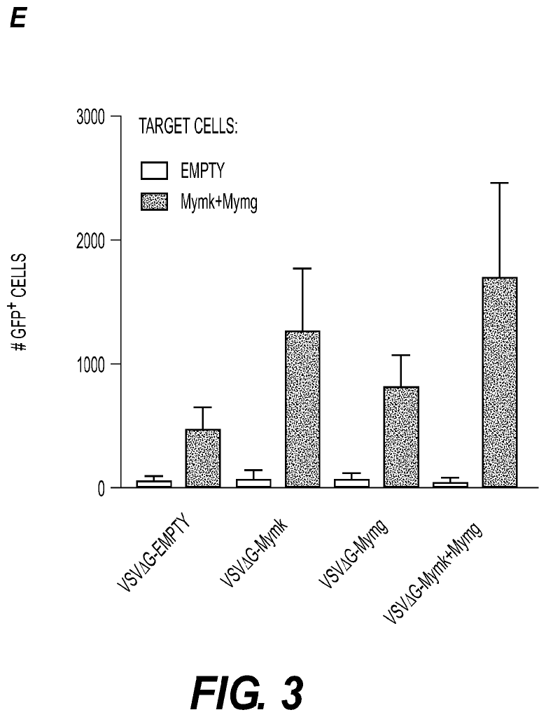

We tested whether the muscle fusogens can be pseudotyped on viral membranes. We utilized a mutant from the vesicular stomatitis virus (VSV). In this system, VSV was re-engineered to contain GFP (Green Fluorescent Protein, which serves as a reporter for successful viral transduction) instead of the native G protein-coding gene responsible for viral membrane fusion (VSVAG). Thus, any fusion capability of the VSVAG must be provided in trans by the membrane from which the virus buds. We pseudotyped Myomaker and/or Myomerger on VSVAG by generating BHK21 cell lines with an inducible expression of the individual muscle fusogens or both as well as an empty vector to serve as a negative control (FIG. 3A, 3B). We verified incorporation of the muscle fusogens on viral membranes by immunoblotting (FIG. 3C). To test the functional capacity of Myomaker and/or Myomerger pseudotyped VSVAG virions (hereafter VSVAG-Mymk, VSVAG-Mymg, VSVAG-Mymk+Mymg, or VSVAG-Bald), we transduced each viral-pseudotype on BHK21 cells expressing empty vector or one-of or both muscle fusogens (FIG. 3D). We observed no signs of transduction by muscle-fusogens viral pseudotypes on empty BHK21 cells or by VSVAG-Bald on Mymk+Mymg expressing BHK21 cells. We observed transduction by VSVAG pseudotyped with muscle fusogens only in the presence of Myomaker+Myomerger on BHK21 with maximum transduction when Myomaker and Myomerger are present on both fusing membranes, i.e., viral and cell membranes (FIG. 3D, 3E). These findings indicate that Myomaker and Myomerger can be pseudotyped on VSVAG and VSVAG-Mymk+Mymg transduces cells with a myogenic fusion profile.

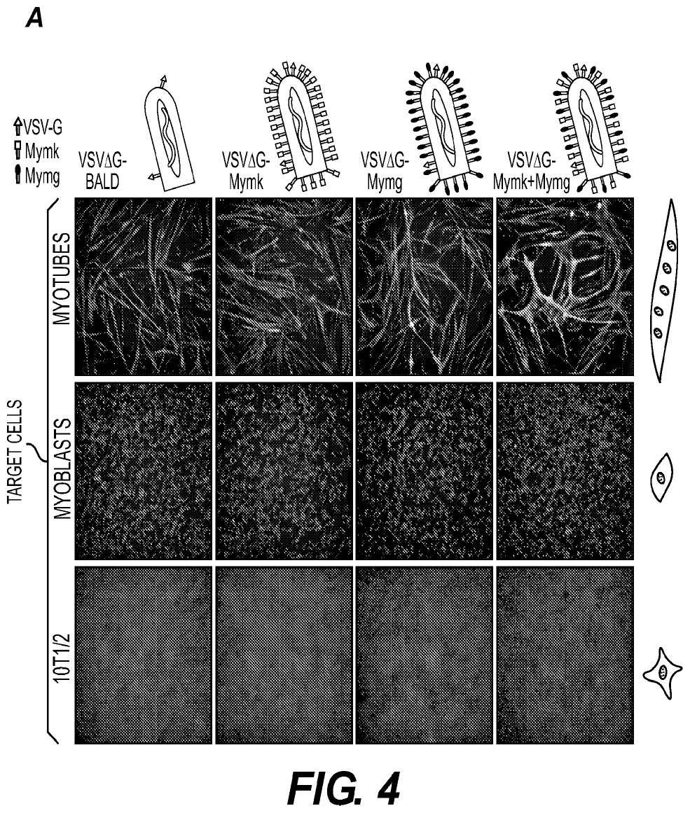

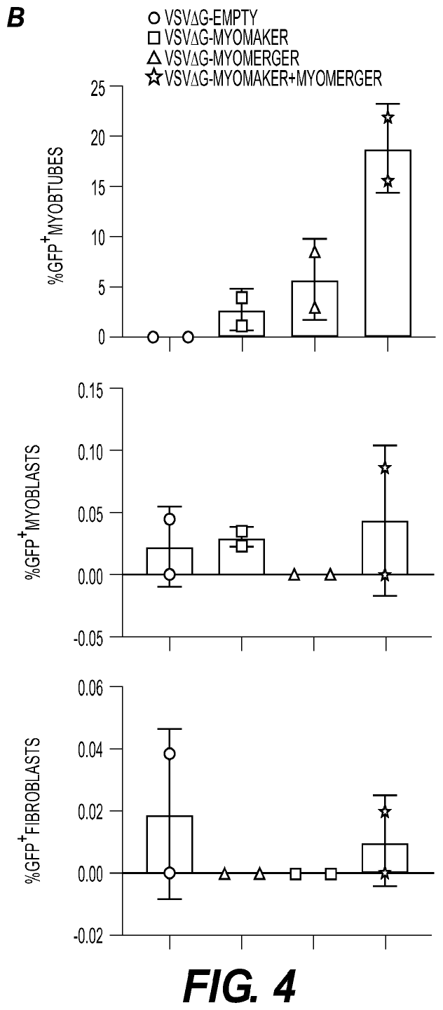

To test whether VSVAG pseudotyped with muscle fusogens can transduce myogenic cells, we infected fusing primary myoblasts as well as proliferating primary myoblasts and 10T½ fibroblasts with the different VSVAG pseudotypes (FIG. 4A). We and only observed transduction by VSVAG-Mymk, VSVAG-Mymg, VSVAG-Mymk+Mymg in fusing myoblasts with highest transduction mediated by VSVAG-Mymk+Mymg (FIG. 4A, 4B) further confirming the specificity of Mymk+Mymg-pseudotyped VSVAG and demonstrating functionality in normal physiological myogenic settings.

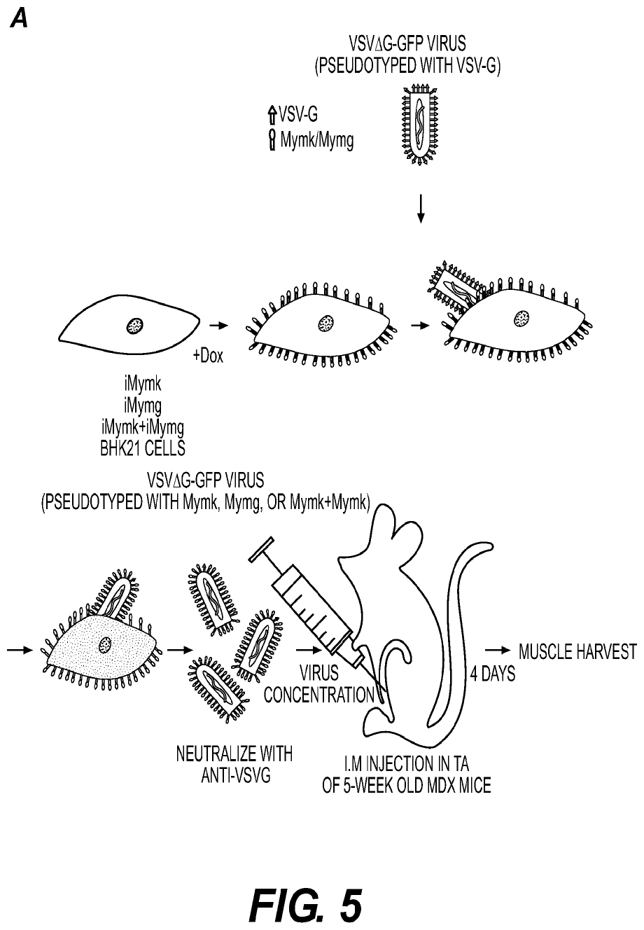

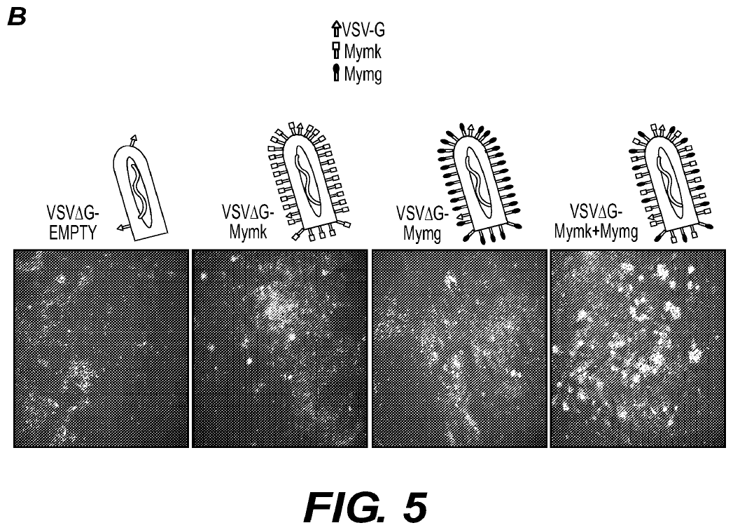

We next investigated if VSVAG particles pseudotyped with muscle fusogens are functional in vivo. Viral concentrates of VSVAG pseudotyped with the individual muscle fusogens or in combination were injected into TA muscle of mdx4cv mice and analyzed 4 days later (FIG. 5A). Similar to our observations in vitro, each of VSVAG-Mymk, VSVAG-Mymg, and VSVAG-Mymk+Mymg demonstrated transduction in vivo as evidenced by GFP expression in the muscle fibers, however, maximum transduction was achieved by VSVAG-Mymk+Mymg (FIG. 5B).

### Example 3—Lentivirus

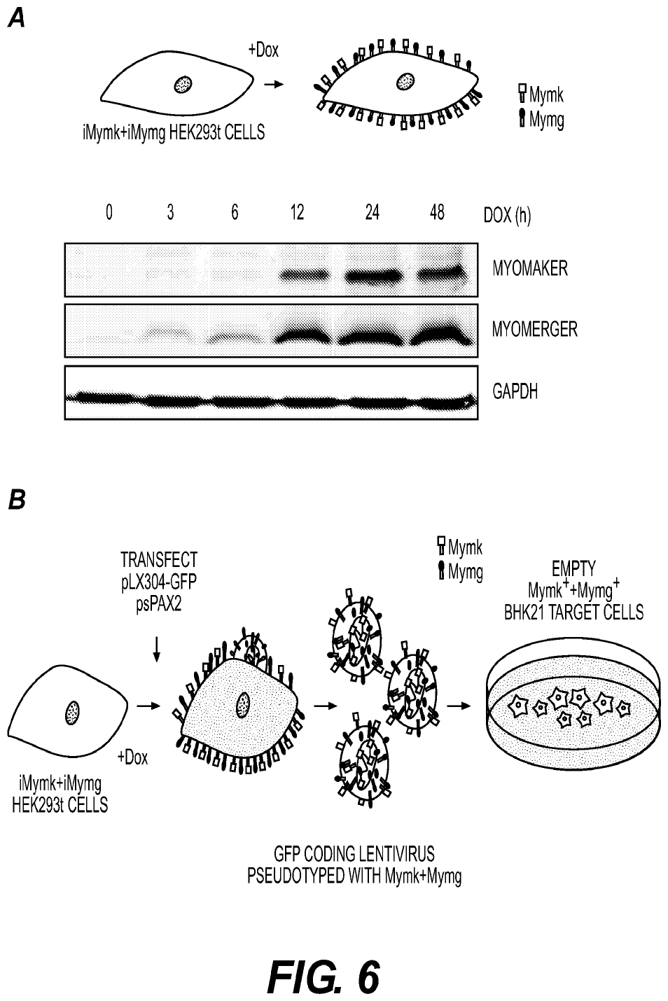

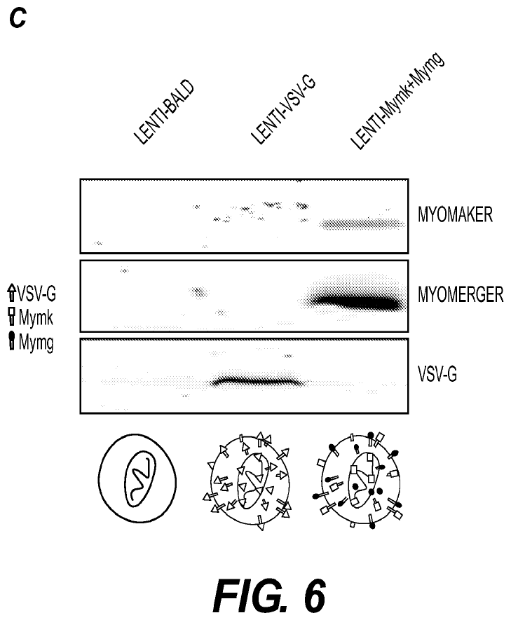

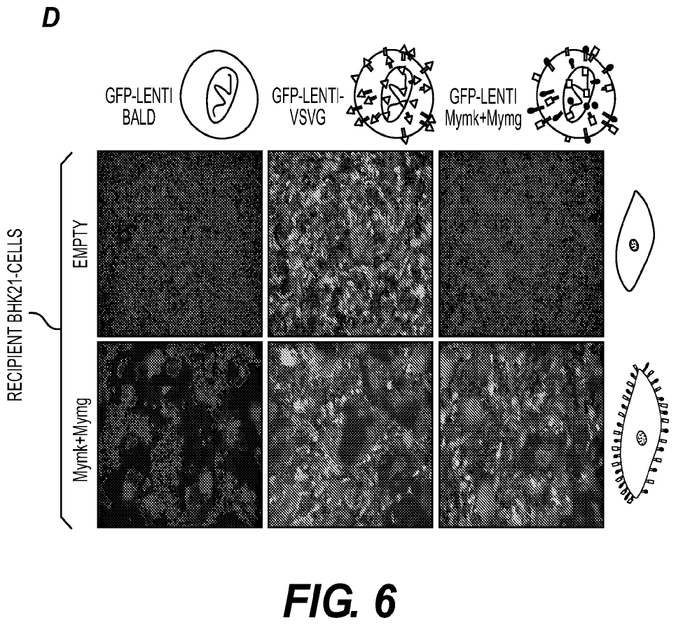

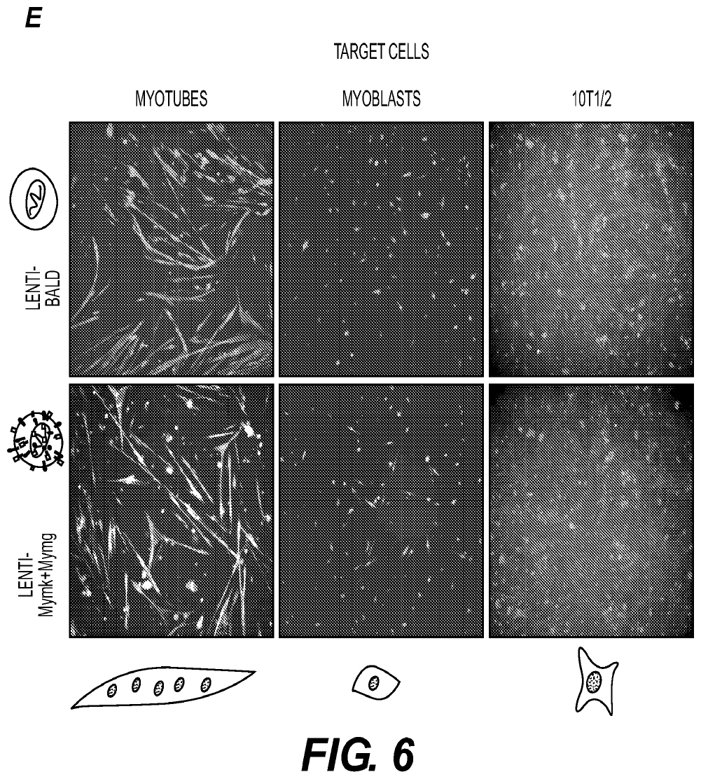

We decided to use lentivirus, another type of enveloped virus, to investigate the utility of another Mymk+Mymg-pseudotyped virus. To facilitate the incorporation of Myomaker and Myomerger on lentivirus, we generated viral producing HEK293t cells with an inducible expression of Myomaker and/or Myomerger (FIG. 6A). We validated the presence of Myomaker and Myomerger on lentivirus particles (FIG. 6B, 6C) and tested their transduction capacity on Empty and Myomaker+Myomerger expressing BHK21 cells (FIG. 6B, 6D). Consistent with our results with Myomaker+Myomerger pseudotyped VSVAG, Lenti-Mymk+Mymg was only able to transduce BHK21 cells in the presence of Myomaker and Myomerger (FIG. 6D). Furthermore, we observed efficient transduction by Lenti-Mymk+Mymg in fusing myoblasts and low but significant transduction in proliferating myoblasts but not 10T/12 fibroblasts (FIG. 6E).

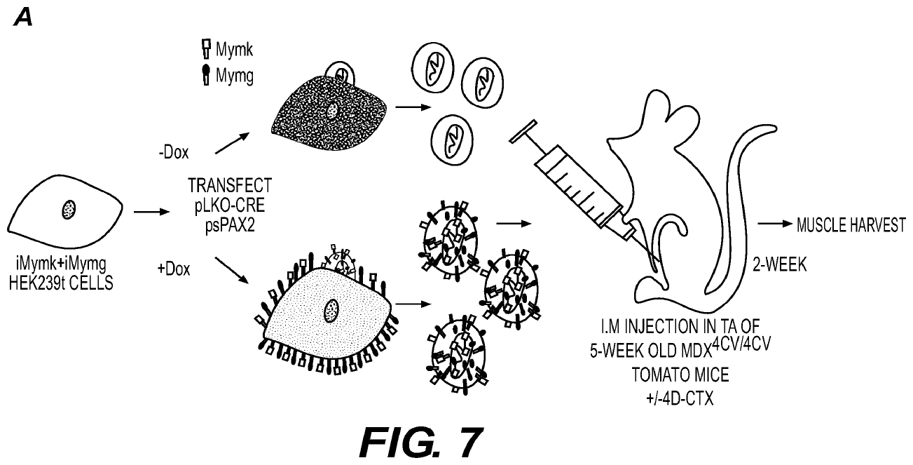

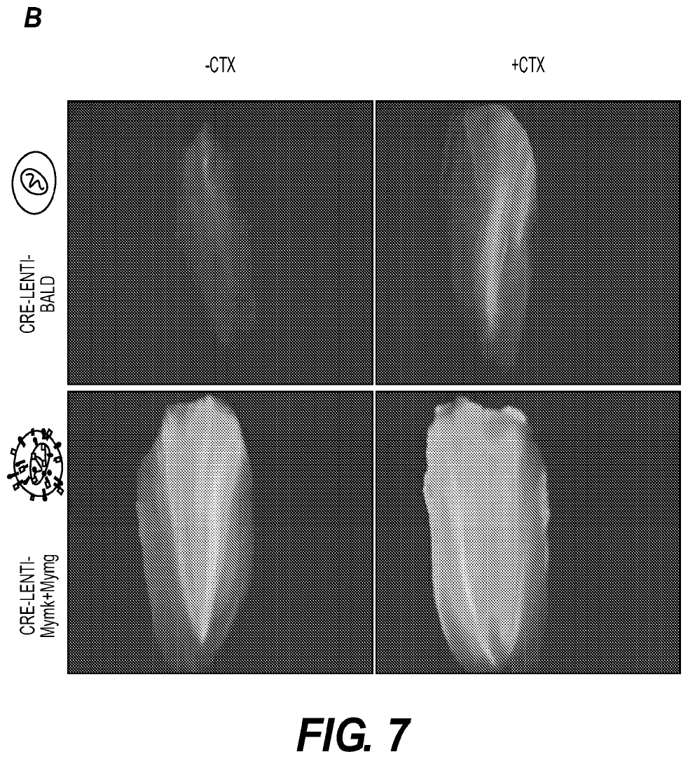

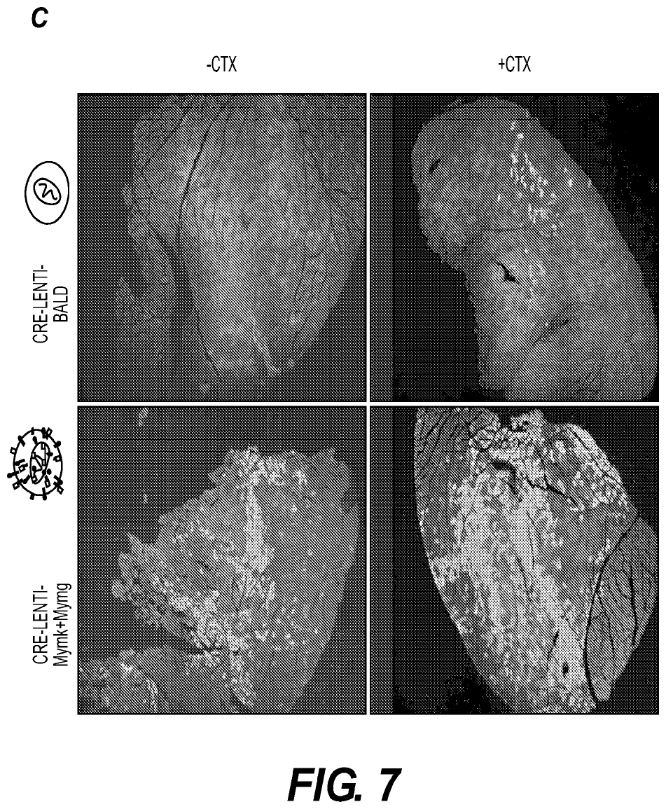

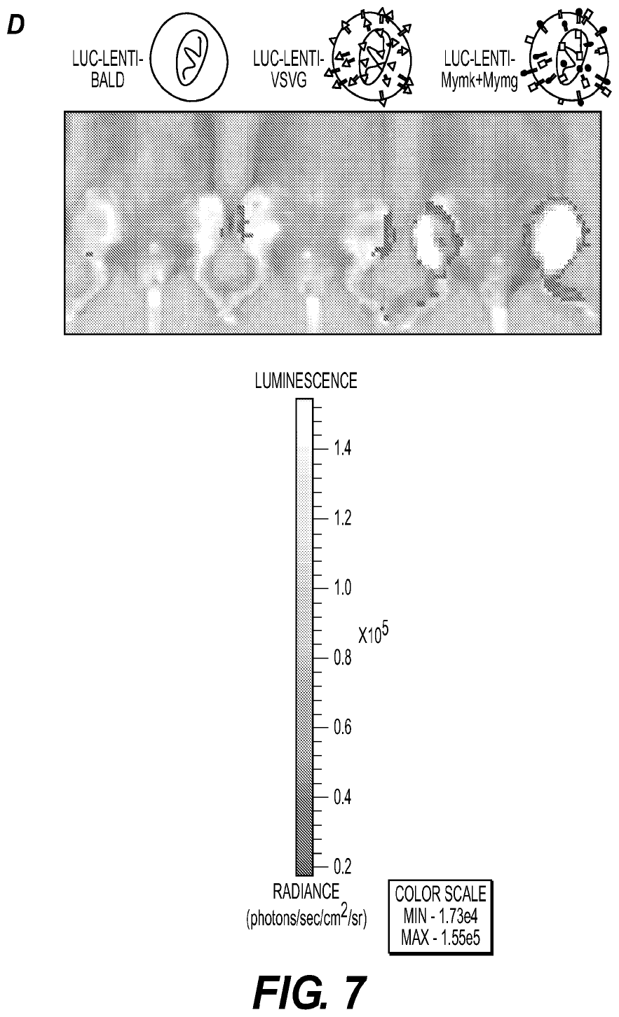

We next sought to investigate if Myomaker+Myomerger pseudotyped lentivirus is capable of transducing myogenic cells in vivo. We generated Mymk+Mymg-pseudotyped Cre-lentivirus and Bald Cre-lentivirus to serve as a negative control (FIG. 7A). Viral supernatants were concentrated and injected into TA muscle of mdx^{4cv};Rosa26^{tdTomato} mice, which harbor a Cre-dependent tdTomato cassette (FIG. 7A). In these mice, tdTomato expression serves as a readout for successful viral transduction. Whole-mount fluorescence imaging of TA muscles of mdx^{4cv};Rosa26^{tdTomato} mice revealed Cre-Lenti-Mymk+Mymg induced tdTomato expression to much higher levels compared to Bald-Lenti-Mymk+Mymg (FIG. 7B). Cross-sections prepared from these muscles revealed that tdTomato is expressed in the muscle fibers (FIG. 7C) indicating that Cre-Lenti-Mymk+Mymg successfully transduced muscle fibers directly or fusing myoblast that in turn fused to regenerating muscle fibers or to each other to form new fibers. Because Myomaker and Myomerger are expressed in myogenic cells undergoing fusion we reasoned that a robust widespread injury to the muscle would increase the number of myogenic cells that are permissive to Cre-Lenti-Mymk+Mymg transduction. Accordingly, we injured TA muscle of mdx^{4cv};Rosa^{26tdTomato} mice with CTX prior to viral injection and analyzed transduction 2-weeks later (FIG. 7A-7C). CTX-injury significantly enhanced the number of fibers expressing tdTomato as evidenced by whole mount and cross-section analysis (FIG. 7B-7C). In a separate experiment, we generated Luciferase-coding lentivirus pseudotyped with Myomaker+Myomerger and compared its transduction capacity to that pseudotyped with VSVG (a conventional lentivirus pseudotyping envelope) following intramuscular injection to injured mdx^{4cv} TA muscle. Luciferase activity was significantly higher in TA muscle injected with Luc-Lenti-Mymk+Mymg as compared to that injected with Luc-Lenti-VSVG (FIG. 7D).

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A pseudotyped particle selected from the group consisting of pseudotyped exosomes and pseudotyped viruses, wherein the pseudotyped particle comprises one or more polypeptides and the one or more polypeptides comprises one or more myomaker polypeptides, one or more myomerger polypeptides, or a combination thereof.

2. The pseudotyped particle of claim 1, wherein one or more myomaker polypeptides, one or more myomerger polypeptides, or a combination thereof is present on the surface of the pseudotyped particle.

3. The pseudotyped particle of any of the preceding claims, wherein one or more myomaker polypeptides, one or more myomerger polypeptides, or a combination thereof is present on the lipid envelope of the pseudotyped particle.

4. The lipid particle of any of the preceding claims, wherein the lipid particle has a size of 20-500 nm, optionally 30-150 nm or 80-120 nm.

5. The pseudotyped particle of any of the preceding claims, wherein the pseudotyped particle does not comprise any nucleic acid encoding a myomaker protein and/or a myomerger protein.

6. The pseudotyped particle of any of the preceding claims, wherein (a) the pseudotyped particle has one or more polypeptides on the pseudotyped particle's surface that are not found in the corresponding naturally occurring particle, (b) the pseudotyped particle has a larger amount of one or more polypeptides on the pseudotyped particle's surface than that found in the corresponding naturally occurring particle, (c) the pseudotyped particle has a larger amount of one or more polypeptides in the pseudotyped particle, by measuring the total amount of polypeptide in the pseudotyped particle, than that found in the corresponding naturally occurring particle, or (d) a combination of (a), (b), or (c).

7. The pseudotyped particle of any of the preceding claims, wherein (a) the pseudotyped particle has one or more polypeptides on the pseudotyped particle's surface that are not found in the corresponding non-pseudotyped particle, (b) the pseudotyped particle has a larger amount of one or more polypeptides on the pseudotyped particle's surface than that found in the corresponding non-pseudotyped particle, (c) the pseudotyped particle has a larger amount of one or more polypeptides in the pseudotyped particle, by measuring the total amount of polypeptide in the pseudotyped particle, than that found in the corresponding non-pseudotyped particle, or (d) a combination of (a), (b), or (c).

8. The pseudotyped particle of any of the preceding claims, wherein the pseudotyped particle is selected from a pseudotyped exosome, a pseudotyped VSV, and a pseudotyped lentivirus.

9. The pseudotyped particle of any of the preceding claims, wherein the pseudotyped particle is a pseudotyped exosome.

10. The pseudotyped particle of any of the preceding claims, wherein the pseudotyped particle is a pseudotyped exosome and the exosome is produced from an exosome producing cell that expresses or overexpresses one or more myomerger polypeptides, one or more myomaker polypeptides, or a combination thereof.

11. The pseudotyped particle of any of the preceding claims, wherein the pseudotyped particle is a pseudotyped exosome and the exosome producing cell is (a) a muscle cell that overexpresses one or more myomerger polypeptides, one or more myomaker polypeptides, or a combination thereof, (b) a myoblast that overexpresses one or more myomerger polypeptides, one or more myomaker polypeptides, or a combination thereof, (c) a myotube that overexpresses one or more myomerger polypeptides, one or more myomaker polypeptides, or a combination thereof, or (d) a fibroblast that expresses one or more myomerger polypeptides, one or more myomaker polypeptides, or a combination thereof.

12. The pseudotyped particle of any of claims 1-8, wherein the pseudotyped particle is a pseudotyped virus.

13. The pseudotyped particle of claim 12, wherein the pseudotyped particle is a pseudotyped Vesicular Stomatitis Virus (VSV).

14. The pseudotyped particle of claim 12, wherein the pseudotyped particle is a pseudotyped VSV and the pseudotyped VSV surface comprises one or more myomerger polypeptides, one or more myomaker polypeptides, or a combination thereof.

15. The pseudotyped particle of any of claims 1-8, wherein the pseudotyped particle is a pseudotyped lentivirus.

16. The pseudotyped particle of claim 15, wherein the pseudotyped particle is a pseudotyped lentivirus and the pseudotyped lentivirus surface comprises one or more myomerger polypeptides, one or more myomaker polypeptides, or a combination thereof.

17. The pseudotyped particle of any of the preceding claims, wherein at least one of the one or more myomerger polypeptides is a wt-myomerger polypeptide.

18. The pseudotyped particle of any of the preceding claims, wherein at least one of the one or more myomerger polypeptides comprises at least one amino acid modification relative to a wt-myomerger polypeptide.

19. The pseudotyped particle of any of the preceding claims, wherein at least one of the one or more myomerger polypeptides comprises at least one amino acid modification relative to a wt-myomerger polypeptide and the at least one amino acid modification is an insertion, a deletion, or a substitution.

20. The pseudotyped particle of any of the preceding claims, wherein at least one of the one or more myomerger polypeptides is selected from the group consisting of SEQ ID NO: 17, SEQ ID NO: 18, SEQ ID NO: 19, SEQ ID NO: 20, SEQ ID NO: 21, SEQ ID NO: 22, and SEQ ID NO: 23.

21. The pseudotyped particle of any of the preceding claims, wherein at least one of the one or more myomerger polypeptides is a human myomerger polypeptide.

22. The pseudotyped particle of any of the preceding claims, wherein at least one of the one or more myomerger polypeptides is SEQ ID NO:19.

23. The pseudotyped particle of any of the preceding claims, wherein at least one of the one or more myomerger polypeptides has at least an 80% sequence identity to a wt-myomerger polypeptide.

24. The pseudotyped particle of any of the preceding claims, wherein at least one of the one or more myomerger polypeptides has at least a 90% sequence identity to a wt-myomerger polypeptide.

25. The pseudotyped particle of any of the preceding claims, wherein at least one of the one or more myomerger polypeptides is an extracellular wt-myomerger polypeptide.

26. The pseudotyped particle of any of the preceding claims, wherein at least one of the one or more myomerger polypeptides comprises (a) amino acids 4-15 of any of SEQ ID Nos: 35-40, (b) amino acids 18-32 of any of SEQ ID Nos: 35-40, or (c) both.

27. The pseudotyped particle of any of the preceding claims, wherein at least one of the one or more myomerger polypeptides comprises (a) LLPLLRRLARRL (SEQ ID NO:41), (b) QDMREALLSCLLFVL (SEQ ID NO:42) or QDMREALLGCLLFIL (SEQ ID NO:43), or (c) both.

28. The pseudotyped particle of any of the preceding claims, wherein at least one of the one or more myomerger polypeptides has at least an 80% sequence identity to an extracellular wt-myomerger polypeptide.

29. The pseudotyped particle of any of the preceding claims, wherein at least one of the one or more myomerger polypeptides has at least a 90% sequence identity to an extracellular wt-myomerger polypeptide.

30. The pseudotyped particle of any of the preceding claims, wherein at least one of the one or more myomaker polypeptides is a wt-myomaker polypeptide.

31. The pseudotyped particle of any of the preceding claims, wherein at least one of the one or more myomaker polypeptides comprises at least one amino acid modification relative to a wt-myomaker polypeptide.

32. The pseudotyped particle of any of the preceding claims, wherein at least one of the one or more myomaker polypeptides comprises at least one amino acid modification relative to a wt-myomaker polypeptide and the at least one amino acid modification is an insertion, a deletion, or a substitution.

33. The pseudotyped particle of any of the preceding claims, wherein at least one of the one or more myomaker polypeptides is selected from the group consisting of SEQ ID NO: 1, SEQ ID NO: 2, SEQ ID NO: 3, SEQ ID NO: 4, SEQ ID NO: 5, and SEQ ID NO: 6.

34. The pseudotyped particle of any of the preceding claims, wherein at least one of the one or more myomaker polypeptides is a human myomerger polypeptide.

35. The pseudotyped particle of any of the preceding claims, wherein at least one of the one or more myomaker polypeptides is SEQ ID NO:1.

36. The pseudotyped particle of any of the preceding claims, wherein at least one of the one or more myomaker polypeptides has at least an 80% sequence identity to a wt-myomaker polypeptide.

37. The pseudotyped particle of any of the preceding claims, wherein at least one of the one or more myomaker polypeptides has at least a 90% sequence identity to a wt-myomaker polypeptide.

38. The pseudotyped particle of any of the preceding claims, wherein the pseudotyped particle comprises one or more myomaker polypeptides and one or more myomerger polypeptides.

39. The pseudotyped particle of any of the preceding claims, wherein the pseudotyped particle further comprises one or more nucleic acid molecules.

40. The pseudotyped particle of any of the preceding claims, wherein the pseudotyped particle does not comprise a myomerger nucleic acid molecule.

41. The pseudotyped particle of any of the preceding claims, wherein the pseudotyped particle does not comprise a myomaker nucleic acid molecule.

42. The pseudotyped particle of any of the preceding claims, wherein the pseudotyped particle does not comprise a myomerger nucleic acid molecule and does not comprise a myomaker nucleic acid molecule.

43. The pseudotyped particle of any of the preceding claims, wherein the pseudotyped particle further comprises a nucleic acid encoding a gene of interest.

44. The pseudotyped particle of any of the preceding claims, wherein the pseudotyped particle further comprises a nucleic acid encoding a gene of interest and the nucleic acid encoding the gene of interest does not encode for a myomaker polypeptide or a myomerger polypeptide.

45. The pseudotyped particle of any of the preceding claims, wherein the pseudotyped particle further comprises a nucleic acid encoding a gene of interest and the gene of interest is a therapeutic gene or a reporter gene.

46. The pseudotyped particle of any of the preceding claims, wherein the pseudotyped particle further comprises a nucleic acid encoding a gene of interest and the gene of interest is a gene for delivery to a muscle cell.

47. The pseudotyped particle of any of the preceding claims, wherein the pseudotyped particle further comprises a nucleic acid encoding a gene of interest and the gene of interest is a dystrophin nucleic acid molecule.

48. The pseudotyped particle of any of the preceding claims, wherein the pseudotyped particle further comprises a nucleic acid encoding a gene of interest and the gene of interest encodes a dystrophin polypeptide.

49. The pseudotyped particle of any of the preceding claims, wherein the pseudotyped particle further comprises a nucleic acid encoding a gene of interest and the gene of interest encodes a microdystrophin or a minidystrophin.

50. The pseudotyped particle of any of the preceding claims, wherein the pseudotyped particle further comprises a nucleic acid that can modulate gene expression.

51. The pseudotyped particle of any of the preceding claims, wherein the pseudotyped particle further comprises a nucleic acid that can modulate gene expression selected from a gRNA/Cas9 and an anti-sense oligonucleotide.

52. The pseudotyped particle of any of the preceding claims, wherein the pseudotyped particle exhibits fusogenic activity with a target cell upon binding of the myomaker polypeptide and/or a myomerger polypeptide to a myomaker polypeptide and/or myomerger polypeptide on the target cell.

53. The pseudotyped particle of claim 52, wherein the target cell endogenously expresses a myomaker polypeptide and/or a myomerger polypeptide, optionally wherein the target cell is a muscle cell.

54. The pseudotyped particle of claim 52, wherein the target cell does not endogenously expresses a myomaker polypeptide and/or a myomerger polypeptide, optionally wherein the target cell is a non-muscle cell.

55. The pseudotyped particle of claim 52, wherein the target cell is a fibroblast, a mesenchymal stem cell (MSC), a hematopoietic stem cell, a blood cell, a bone marrow cell, or an adipose stem cell.

56. A method for producing a pseudotyped lentivirus comprising
contacting, in any order, (a) a composition comprising cells that express at least one myomerger polypeptide, at least one myomaker polypeptide, or a combination thereof, (b) a composition comprising one or more lentivirus production plasmids, (c) a composition comprising a nucleic acid encoding a gene of interest and/or a nucleic acid that modulates gene expression, and (d) optionally, a composition comprising one or more chemicals to increase transfection or transduction efficiency; and
optionally, recovering the pseudotyped lentivirus.

57. The method of claim 56, wherein the compositions of (b) and/or (c) are contacted with the composition of (d), and then the (b) and/or (c) with (d) mixture is contacted with the composition of (a).

58. The method of any of claims 56-57, wherein (i) the cells inducibly express at least one myomerger polypeptide, at least one myomaker polypeptide, or a combination thereof and (ii) an induction chemical is added to the composition of (a) prior to contacting (b) or (c) or (d), after (a) contacts (b), after (a) contacts (c), after (a) contacts (d), after (a) contacts (b) and/or (c) with (d), or a combination thereof.

59. The method of any of claims 56-58, wherein (i) the cells inducibly express at least one myomerger polypeptide, at least one myomaker polypeptide, or a combination thereof and (ii) an induction chemical is added to the composition of (a) prior to contacting (b) or (c) or (d).

60. The method of any of claims 56-59, wherein the one or more lentivirus production plasmids comprise one or more transfer plasmids, one or more packaging plasmids, or a combination thereof.

61. The method of any of claims 56-60, wherein the one or more lentivirus production plasmids comprise transfer plasmid plX304-GFP and packaging plasmid psPAX2.

62. The method of any of claims 56-61, wherein the recovering step comprises centrifugation, filtration, ultracentrifugation, ultrafiltration, chromatography, or hydrophobic interaction chromatography, or a combination thereof.

63. A method for producing a pseudotyped VSV comprising
contacting, in any order, (a) a composition comprising cells that express at least one myomerger polypeptide, at least one myomaker polypeptide, or a combination thereof, (b) a composition comprising one or more VSV production plasmids, (c) a composition comprising a nucleic acid encoding a gene of interest and/or a nucleic acid that modulates gene expression, and (d) optionally, a composition comprising one or more chemicals to increase transfection or transduction efficiency; and
optionally, recovering the pseudotyped VSV.

64. The method of claim 63, wherein the recovering step comprises centrifugation, filtration, ultracentrifugation, ultrafiltration, chromatography, or hydrophobic interaction chromatography, or a combination thereof.

65. A method of producing a pseudotyped exosome comprising (a) growing exosome producing cells that express at least one myomerger polypeptide, at least one myomaker polypeptide, a polypeptide of interest, or a combination thereof, (b) optionally contacting the exosome producing cells with a nucleic acid of interest, the polypeptide of interest, or a combination thereof, and (c) placing the exosome producing cells in an exosome depleted media.

66. The method of claim 65, wherein the method further comprises recovering the pseudotyped exosomes.

67. The method of claim 65, wherein the method further comprises recovering the pseudotyped exosomes and the recovering step comprises centrifugation, filtration, ultracentrifugation, ultrafiltration, chromatography, or hydrophobic interaction chromatography, or a combination thereof.

68. A pseudotyped lentivirus produced by the method of any of claims 56-62.

69. A pseudotyped VSV produced by the method of any of claims 63-64.

70. A pseudotyped exosome produced by the method of any of claims 65-67.

71. A modified cell suitable to produce the pseudotyped particle of any claims 1-55,68-70.

72. A modified cell comprising one or more nucleic acids encoding proteins for pseudotyped particle production and nucleic acid encoding a myomaker polypeptide and/or a myomerger polypeptide.

73. The modified cell of claim 72, wherein the encoded myomaker polypeptide and/or myomerger polypeptide is transiently expressed by the modified cell.

74. The modified cell of any of claims 72-73, wherein the encoded myomaker polypeptide and/or myomerger polypeptide is expressed from a plasmid.

75. The modified cell of claim 72, wherein the encoded myomaker polypeptide and/or myomerger polypeptide is stably expressed by the modified cell.

76. The modified cell of claim 72 or claim 75, wherein the encoded myomaker polypeptide and/or myomerger polypeptide is expressed from an endogenous locus.

77. The modified cell of any of claims 71-76, wherein the myomaker polypeptide and/or myomerger polypeptide is overexpressed.

78. The modified cell of any of claims 71-77, wherein the encoded myomaker polypeptide and/or myomerger polypeptide is inducibly expressed by the modified cell.

79. The modified cell of claim 78, wherein the nucleic acid encoding a myomaker polypeptide and/or a myomerger polypeptide is linked to an inducible response element, optionally a promoter.

80. A modified cell comprising a nucleic acid encoding a myomaker polypeptide and/or a myomerger polypeptide, wherein the nucleic acid encoding the myomaker polypeptide and/or the myomerger polypeptide is linked to an inducible response element, optionally a promoter.

81. The modified cell of claim 79 or claim 80, wherein the inducible response element is a doxycycline response element.

82. The modified cell of any of claims 71-81, wherein the modified cell overexpresses a myomaker polypeptide and overexpresses a myomerger polypeptide.

83. The modified cell of any of claims 71-82, wherein the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a BHK21 cell, a modified BHK21 cell, a HEK293t cell, a modified HEK293t cell, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

84. The modified cell of any of claims 71-83, wherein the modified cell is a modified myoblast, a modified fibroblast, a BHK21 cell, a modified BHK21 cell, a HEK293t cell, a modified HEK293t cell, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

85. A composition comprising the pseudotyped particle of any of claims 1-55, 68-70 or the modified cell of any of claims 71-84.

86. The composition of claim 85, wherein the amount of the pseudotyped particle or the modified cell is from about 0.0001% (by weight total composition) to about 99%.

87. A pharmaceutical composition comprising the pseudotyped particle of any of claims 1-55, 68-70 or the modified cell of any of claims 71-84.

88. The pharmaceutical composition of claim 87, wherein the amount of the pseudotyped particle or the modified cell is from about 0.0001% (by weight total composition) to about 50%.

89. A method for mediating fusion of a pseudotyped particle with a target cell, the method comprising contacting the target cell with the pseudotyped particle of any of claims 1-55, 68-70.

90. A method of delivering a gene of interest to a target cell, the method comprising contacting the pseudotyped particle of any of claims 1-55, 68-70 with a target cell.

91. A method of delivering a gene that modulates gene expression to a target cell, the method comprising contacting the pseudotyped particle of any of claims 1-55, 68-70 with a target cell.

92. The method of any of claims 89-91, wherein the contacting occurs in vitro or in vivo.

93. The method of any of claims 89-92, wherein the target cell endogenously expresses a myomaker polypeptide and/or a myomerger polypeptide and optionally the target cell is a muscle cell.

94. The method of any of claims 89-93, wherein the target cell is a muscle cell, a myoblast, a myotube, or a mesenchymal stem cell (MSC).

95. The method of any of claims 89-92, wherein the target cell does not endogenously express a myomaker polypeptide and/or a myomerger polypeptide and optionally the target cell is a non-muscle cell.

96. The method of claim 95, wherein the target cell is a non-muscle cell, a fibroblast, a mesenchymal stem cell (MSC), a hematopoietic stem cell, a blood cell, a bone marrow cell, or an adipose stem cell.

97. The method of claim 89-96, wherein the target cell under expresses dystrophin, does not express dystrophin, or expresses a defective form of dystrophin.

98. A method for administering a pseudotyped particle to an animal comprising
administering the pseudotyped particle to the animal;
wherein
the pseudotyped particle of any of claims 1-55, 68-70.

99. The method of claim 98, wherein the administering is part of treating a disease.

100. A method for treating a disease in an animal comprising administering the pseudotyped particle of any of claims 1-55, 68-70 to the animal.

101. The method of any of claims 98-100, wherein the pseudotyped particle comprises a nucleic acid molecule comprising a gene of interest.

102. The method of any of claims 98-101, wherein the gene of interest encodes a dystrophin polypeptide.

103. The method of any of claims 98-102, wherein the gene of interest encodes a microdystrophin or a minidystrophin.

104. The method of any of claims 98-103, wherein the pseudotyped particle comprises a nucleic acid molecule that can modulate gene expression.

105. The method of any of claims 98-104, wherein the pseudotyped particle comprises a nucleic acid molecule that can modulate gene expression selected from gRNA/Cas9 and anti-sense oligonucleotides.

106. The method of any of claims 98-105, wherein the pseudotyped particle is a pseudotyped exosome.

107. The method of any of claims 98-106, wherein the pseudotyped particle is a pseudotyped exosome and the pseudotyped exosome comprises a dystrophin polypeptide.

108. The method of any of claims 98-105, wherein the pseudotyped particle is a pseudotyped VSV.

109. The method of any of claims 98-105, 108, wherein the pseudotyped particle is a pseudotyped VSV and the pseudotyped VSV comprises a nucleic acid molecule comprising a gene of interest.

110. The method of any of claims 98-105, 108-109, wherein the pseudotyped particle is a pseudotyped VSV and the pseudotyped VSV comprises a nucleic acid molecule comprising a dystrophin nucleic acid molecule.

111. The method of any of claims 98-105, 108-110, wherein the pseudotyped particle is a pseudotyped VSV and the pseudotyped VSV comprises a nucleic acid molecule that can modulate gene expression.

112. The method of any of claims 98-105, wherein the pseudotyped particle is a pseudotyped lentivirus.

113. The method of any of claims 98-105, 112, wherein the pseudotyped particle is a pseudotyped lentivirus and the pseudotyped lentivirus comprises a nucleic acid molecule comprising a gene of interest.

114. The method of any of claims 98-105, 112-113, wherein the pseudotyped particle is a pseudotyped lentivirus and the pseudotyped lentivirus comprises a nucleic acid molecule comprising a dystrophin nucleic acid molecule.

115. The method of any of claims 98-105, 112-114, wherein the pseudotyped particle is a pseudotyped lentivirus and the pseudotyped lentivirus comprises a nucleic acid molecule that can modulate gene expression.

116. The method of any of claims 98-115, wherein the administering is parenteral administration, mucosal administration, intravenous administration, depot injection, subcutaneous administration, topical administration, intradermal administration, oral administration, sublingual administration, intranasal administration, or intramuscular administration.

117. The method of any of claims 98-116, wherein the administering is an injection or an intramuscular injection.

118. The method of any of claims 98-117, wherein the animal is selected from mammals, primates, monkeys, macaque, rhesus macaque, or pig tail macaque, humans, canine, feline, bovine, porcine, avian, chicken, mice, rabbits, and rats.

119. The method of any of claims 98-118, wherein the animal is a mouse, rat, or human.

120. The method of any of claims 98-119, wherein the disease is a muscle related disease.

121. The method of any of claims 98-120 wherein the disease is a disease where the animal's cells under express dystrophin, do not express dystrophin, or express a defective form of dystrophin.

122. The method of any of claims 98-121, wherein the disease is myopathy, muscular dystrophy, amyotrophic lateral sclerosis (ALS or also called Lou Gehrig's disease), glycogen storage disease type II (also called Pompe disease), rhabdomyosarcoma (RMS), or sarcopenia.

123. The method of any of claims 98-122, wherein the disease is muscular dystrophy.

124. The method of any of claims 98-123, wherein the animal is in need of treatment of a disease.
